SEQUENCE LISTING (49 sequences)

SEQ ID NO: 1 - DNA, 360 bp, Artificial Sequence
  Synthetic sequence Anti-STII mAb 3E8 VH
gaggtgcagctggtggagactgggggaggctttgtgaagcctggaggctccctgaaactctcctgtgcagcctctggattcactttcagtagttatggcatgtcttgggttcgccagactccggagaagaggctggagtgggtcgcagccatcaccagtgatggcggtggcacccactatccagatactgtgaagggccgattcaccatctccagagactttgccaaaaacaccctgtacctgcagatgagcagtctgaggtctgaggacacagcctggtatttctgtgcaagacatgagccccgactgatagcctggtttgctcactggggccaaggaactctggtcactgtctctgca

SEQ ID NO: 2 - protein, 120 aa, Artificial Sequence
  Synthetic sequence Anti-STII mAb 3E8 VH
EVQLVETGGGFVKPGGSLKLSCAASGFTFSSYGMSWVRQTPEKRLEWVAAITSDGGGTHYPDTVKGRFTISRDFAKNTLYLQMSSLRSEDTAWYFCARHEPRLIAWFAHWGQGTLVTVSA

SEQ ID NO: 3 - protein, 112 aa, Artificial Sequence
  Synthetic sequence Anti-STII mAb 3E8 VL
DVLMTQTPLSLPVSLGDQASISCRSSQSIVHSNGYTYLEWYLQKPGQSPKLLIYEVSNRFSGVPDRFSGSGSGTDFTLKIIRVEAEDLGVYYCFQGSHVPWTFGGGTKLEIK

SEQ ID NO: 4 - DNA, 337 bp, Artificial Sequence
  Synthetic sequence Anti-STII mAb 3E8 VL
gatgttttgatgacccaaactccactctccctgcctgtcagtcttggagatcaagcctctatctcttgcagatctagtcagagcattgttcatagtaatggatacacctatttagaatggtacctgcagaaaccaggccagtctccaaagctcctgatctacgaagtttccaaccgattttctggggtcccagacaggttcagtggcagtggatcagggacagatttcacactcaagatcatcagagtggaggctgaggatctgggagtttattattgctttcaaggttcacatgttccgtggacgttcggtggaggcaccaagctggaaatcaaac

SEQ ID NO: 5 - protein, 247 aa, Artificial Sequence
  Synthetic sequence Anti-STII 3E8 scFv
EVQLVETGGGFVKPGGSLKLSCAASGFTFSSYGMSWVRQTPEKRLEWVAAITSDGGGTHYPDTVKGRFTISRDFAKNTLYLQMSSLRSEDTAWYFCARHEPRLIAWFAHWGQGTLVTVSAGGGGSGGGGSGGGGSDVLMTQTPLSLPVSLGDQASISCRSSQSIVHSNGYTYLEWYLQKPGQSPKLLIYEVSNRFSGVPDRFSGSGSGTDFTLKIIRVEAEDLGVYYCFQGSHVPWTFGGGTKLEIK

SEQ ID NO: 6 - protein, 247 aa, Artificial Sequence
  Synthetic sequence Anti-STII 3E8 scFv
DVLMTQTPLSLPVSLGDQASISCRSSQSIVHSNGYTYLEWYLQKPGQSPKLLIYEVSNRFSGVPDRFSGSGSGTDFTLKIIRVEAEDLGVYYCFQGSHVPWTFGGGTKLEIKGGGGSGGGGSGGGGSEVQLVETGGGFVKPGGSLKLSCAASGFTFSSYGMSWVRQTPEKRLEWVAAITSDGGGTHYPDTVKGRFTISRDFAKNTLYLQMSSLRSEDTAWYFCARHEPRLIAWFAHWGQGTLVTVSA

SEQ ID NO: 7 - DNA, 360 bp, Artificial Sequence
  Synthetic sequence Anti-STII mAb 5G2 VH
caggttcaactgcagcagtctggagctgagctggcgaggccaggggcttcagtgaagctgtcctgcacggcttctggatacaccttcacaagctatggtataacctgggtgaggcagagaactggacagggccttgagtggattggagagatttttcctggaagtggtgatacttcctacggtgagaaattcaagggccaggccacactgactacagacaaatcctccagcacagcctacatgcagctcagcagcctgacatctgaggactctgcagtctatttctgtgcaagacgctataggtacatttaccatgctatggactactggggtcaaggaacctcagtcaccgtctcctca

SEQ ID NO: 8 - protein, 120 aa, Artificial Sequence
  Synthetic sequence Anti-STII mAb 5G2 VH
QVQLQQSGAELARPGASVKLSCTASGYTFTSYGITWVRQRTGQGLEWIGEIFPGSGDTSYGEKFKGQATLTTDKSSSTAYMQLSSLTSEDSAVYFCARRYRYIYHAMDYWGQGTSVTVSS

SEQ ID NO: 9 - DNA, 336 bp, Artificial Sequence
  Synthetic sequence Anti-STII mAb 5G2 VL
gatattttgatgacccaaactccactctccctgcctgtcagtcttggagatcaagcctccatctcttgcagatctagtcagagcattgtacatagtaatggcaacacctatttagaatggtacctgcagaaaccaggccagtctccaaagctcctgatctacaaagtttccaaccgattttctggggtcccagacaggttcagtggcagtggatcagggacagatttcacactcaagatccgcagagtggaggctgaggatctgggagtttattactgctttcaaggttcacatgttccgctcacgttcggtgctgggaccaagctggagctgaaa

SEQ ID NO: 10 - protein, 112 aa, Artificial Sequence
  Synthetic sequence Anti-STII mAb 5G2 VL
DILMTQTPLSLPVSLGDQASISCRSSQSIVHSNGNTYLEWYLQKPGQSPKLLIYKVSNRFSGVPDRFSGSGSGTDFTLKIRRVEAEDLGVYYCFQGSHVPLTFGAGTKLELK

SEQ ID NO: 11 - protein, 247 aa, qArtificial Sequence
  Synthetic sequence Anti-STII 5G2 scFv
QVQLQQSGAELARPGASVKLSCTASGYTFTSYGITWVRQRTGQGLEWIGEIFPGSGDTSYGEKFKGQATLTTDKSSSTAYMQLSSLTSEDSAVYFCARRYRYIYHAMDYWGQGTSVTVSSGGGGSGGGGSGGGGSDILMTQTPLSLPVSLGDQASISCRSSQSIVHSNGNTYLEWYLQKPGQSPKLLIYKVSNRFSGVPDRFSGSGSGTDFTLKIRRVEAEDLGVYYCFQGSHVPLTFGAGTKLELK

SEQ ID NO: 12 - protein, 247 aa, Artificial Sequence
  Synthetic sequence Anti-STII 5G2 scFv
DILMTQTPLSLPVSLGDQASISCRSSQSIVHSNGNTYLEWYLQKPGQSPKLLIYKVSNRFSGVPDRFSGSGSGTDFTLKIRRVEAEDLGVYYCFQGSHVPLTFGAGTKLELKGGGGSGGGGSGGGGSQVQLQQSGAELARPGASVKLSCTASGYTFTSYGITWVRQRTGQGLEWIGEIFPGSGDTSYGEKFKGQATLTTDKSSSTAYMQLSSLTSEDSAVYFCARRYRYIYHAMDYWGQGTSVTVSS

SEQ ID NO: 13 - DNA, 360 bp, Artificial Sequence
  Synthetic sequence Anti-STII mAb 4E2 VH
caggttcaactgcagcagtctggagctgagctggcgaggccaggggcttcagtgaagctgtcctgcacggcttctggatacaccttcacaagctatggtataacctgggtgaggcagagaactggacagggccttgagtggattggagagatttttcctggaagtggtgatacttcctacggtgagaaattaaagggccaggccacactgactacagacaaatcctccagcacagcctacatgcagctcagcagcctgacatctgaggactctgcagtctatttctgtgcaagacgctataggtacatttaccatgctatggactactggggtcaaggaacctcagtcaccgtctcctca

SEQ ID NO: 14 - protein, 120 aa, Artificial Sequence
  Synthetic sequence Anti-STII mAb 4E2 VH
QVQLQQSGAELARPGASVKLSCTASGYTFTSYGITWVRQRTGQGLEWIGEIFPGSGDTSYGEKLKGQATLTTDKSSSTAYMQLSSLTSEDSAVYFCARRYRYIYHAMDYWGQGTSVTVSS

SEQ ID NO: 15 - DNA, 336 bp, Artificial Sequence
  Synthetic sequence Anti-STII mAb 4E2 VL
gatattttgatgacccaaactccactctccctgcctgtcagtcttggagatcaagcctccatctcttgcagatctagtcagagcattgtacatagtaatggcaacacctatttagagtggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacatgttccgctcacgttcggtgctgggaccaagctggagctgaaa

SEQ ID NO: 16 - protein, 112 aa, Artificial Sequence
  Synthetic sequence Anti-STII mAb 4E2 VL
DILMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPLTFGAGTKLELK

SEQ ID NO: 17 - protein, 247 aa, Artificial Sequence
  Synthetic sequence Anti-STII 4E2 scFv
QVQLQQSGAELARPGASVKLSCTASGYTFTSYGITWVRQRTGQGLEWIGEIFPGSGDTSYGEKLKGQATLTTDKSSSTAYMQLSSLTSEDSAVYFCARRYRYIYHAMDYWGQGTSVTVSSGGGGSGGGGSGGGGSDILMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPLTFGAGTKLELK

SEQ ID NO: 18 - protein, 247 aa, Artificial Sequence
  Synthetic sequence Anti-STII 4E2 scFv
DILMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPLTFGAGTKLELKGGGGSGGGGSGGGGSQVQLQQSGAELARPGASVKLSCTASGYTFTSYGITWVRQRTGQGLEWIGEIFPGSGDTSYGEKLKGQATLTTDKSSSTAYMQLSSLTSEDSAVYFCARRYRYIYHAMDYWGQGTSVTVSS

SEQ ID NO: 19 - protein, 8 aa, Artificial Sequence
  Synthetic sequence Strep-Tag II
WSHPQFEK

SEQ ID NO: 20 - protein, 10 aa, Artificial Sequence
  Synthetic sequence (Gly4Ser)2 linker
GGGGSGGGGS

SEQ ID NO: 21 - protein, 8 aa, Artificial Sequence
  Synthetic sequence (Gly3Ser)2 linker
GGGSGGGS

SEQ ID NO: 22 - protein, 13 aa, Artificial Sequence
  Synthetic sequence 3E8 HCDR1 amino acid
AASGFTFSSYGMS

SEQ ID NO: 23 - protein, 10 aa, Artificial Sequence
  Synthetic sequence 3E3 HCDR2 amino acid
AITSDGGGTH

SEQ ID NO: 24 - protein, 13 aa, Artificial Sequence
  Synthetic sequence 3E8 HCDR3
ARHEPRLIAWFAH

SEQ ID NO: 25 - protein, 16 aa, Artificial Sequence
  Synthetic sequence 3E8 LCDR1
RSSQSIVHSNGYTYLE

SEQ ID NO: 26 - protein, 8 aa, Artificial Sequence
  Synthetic sequence 3E8 LCDR2
YEVSNRFS

SEQ ID NO: 27 - protein, 9 aa, Artificial Sequence
  Synthetic sequence 3E8 LCDR3
FQGSHVPWT

SEQ ID NO: 28 - protein, 13 aa, Artificial Sequence
  Synthetic sequence 5G2 HCDR1
TASGYTFTSYGIT

SEQ ID NO: 29 - protein, 10 aa, Artificial Sequence
  Synthetic sequence 5G2 HCDR2
EIFPGSGDTS

SEQ ID NO: 30 - protein, 13 aa, Artificial Sequence
  Synthetic sequence 5G2 HCDR3
ARRYRYIYHAMDY

SEQ ID NO: 31 - protein, 16 aa, Artificial Sequence
  Synthetic sequence 5G2 LCDR1
RSSQSIVHSNGNTYLE

SEQ ID NO: 32 - protein, 8 aa, Artificial Sequence
  Synthetic sequence 5G2 LCDR2
YKVSNRFS

SEQ ID NO: 33 - protein, 9 aa, Artificial Sequence
  Synthetic sequence 5G2 LCDR3
FQGSHVPLT

SEQ ID NO: 34 - protein, 13 aa, Artificial Sequence
  Synthetic sequence 4E2 HCDR1
TASGYTFTSYGIT

SEQ ID NO: 35 - protein, 10 aa, Artificial Sequence
  Synthetic sequence 4E2 HCDR2
EIFPGSGDTS

SEQ ID NO: 36 - protein, 13 aa, Artificial Sequence
  Synthetic sequence 4E2  HCDR3
ARRYRYIYHAMDY

SEQ ID NO: 37 - protein, 16 aa, Artificial Sequence
  Synthetic sequence 4E2  LCDR1
RSSQSIVHSNGNTYLE

SEQ ID NO: 38 - protein, 8 aa, Artificial Sequence
  Synthetic sequence 4E2 LCDR2
YKVSNRFS

SEQ ID NO: 39 - protein, 9 aa, Artificial Sequence
  Synthetic sequence 4E2  LCDR3
FQGSHVPLT

SEQ ID NO: 40 - protein, 19 aa, Artificial Sequence
  Synthetic sequence P2A
ATNFSLLKQAGDVEENPGP

SEQ ID NO: 41 - protein, 22 aa, Artificial Sequence
  Synthetic sequence P2A mod
GSGATNFSLLKQAGDVEENPGP

SEQ ID NO: 42 - protein, 18 aa, Artificial Sequence
  Synthetic sequence T2A
EGRGSLLTCGDVEENPGP

SEQ ID NO: 43 - protein, 21 aa, Artificial Sequence
  Synthetic sequence T2A mod
GSGEGRGSLLTCGDVEENPGP

SEQ ID NO: 44 - protein, 21 aa, Artificial Sequence
  Synthetic sequence E2A
QCTNYALLKLAGSDVESNPGP

SEQ ID NO: 45 - protein, 24 aa, Artificial Sequence
  Synthetic sequence E2A mod
GSGQCTNYALLKLAGSDVESNPGP

SEQ ID NO: 46 - protein, 22 aa, Artificial Sequence
  Synthetic sequence F2A
VKQTLNFDLLKLAGDVESNPGP

SEQ ID NO: 47 - protein, 25 aa, Artificial Sequence
  Synthetic sequence F2A mod
GSGVKQTLNFDLLKLAGDVESNPGP

SEQ ID NO: 48 - protein, 8 aa, Artificial Sequence
  Synthetic sequence Strep-tag
WRHPQFGG

SEQ ID NO: 49 - protein, 11 aa, Artificial Sequence
  Synthetic sequence (Gly3Ser)2Gly2Ser linker
GGGSGGGSGGS

(19) United States
(12) Patent Application Publication
(10) Pub. No.: US 2021/0023132 A1
(43) Pub. Date: Jan. 28, 2021

(54) STREP-TAG SPECIFIC CHIMERIC RECEPTORS AND USES THEREOF

(71) Applicants: FRED HUTCHINSON CANCER RESEARCH CENTER, Seattle, WA (US); TECHNISCHE UNIVERSITÄT MÜNCHEN, München (DE)

(72) Inventors: Lingfeng LIU, Seattle, WA (US); Stanley R. RIDDELL, Sammamish, WA (US); Dirk BUSCH, Schliersee (DE); Simon Fraessle, Freising (DE)

(21) Appl. No.: 16/644,947

(22) PCT Filed: Sep. 6, 2018
(86) PCT No.: PCT/US2018/049804

§ 371 Date: Mar. 5, 2020

Related U.S. Application Data
(60) Provisional application No. 62/555,012, filed on Sep. 6, 2017.

(51) Int. Cl. A61K 35/17; C07K 14/705; C07K 14/725

(52) U.S. Cl. CPC A61K 35/17; C07K 14/70578; C07K 14/7051; C07K 2317/73; C07K 2319/03; C07K 2317/565; C07K 2317/622

(57) ABSTRACT

The present disclosure provides tag-specific fusion proteins for selectively detecting molecules containing a strep-tag peptide or cells containing a strep-tag peptide. Disclosed embodiments include tag-specific fusion proteins that can be used in reagents and methods for monitoring and/or modulating immunotherapy cells that express a strep-tag peptide. Embodiments including fusion proteins that specifically bind tagged targets and recombinant host cells comprising polynucleotides encoding the tag-specific fusion proteins are also provided. Immunotherapy cells that express a tagged marker are also provided.

### CROSS-REFERENCE TO RELATED APPLICATION(S)

This application claims the priority benefit of U.S. patent application No. 62/555,012, filed Sep. 6, 2017, which is incorporated herein by reference for all purposes as if fully set forth herein.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 360056_450WO_SEQUENCE_LISTING.txt. The text file is 28.9 KB, was created on Sep. 3, 2018, and is being submitted electronically via EFS-Web.

### BACKGROUND

Adoptive transfer of genetically modified T cells has emerged as a potent therapy for various malignancies. The most widely employed strategy has been infusion of patient-derived T cells expressing chimeric antigen receptors (CARs) targeting tumor associated antigens. This approach has numerous theoretical advantages, including the ability to target T cells to any cell surface antigen, circumvent loss of major histocompatibility complex as a tumor escape mechanism, and employ a single vector construct to treat any patient, regardless of human leukocyte antigen haplotype. For example, CAR clinical trials for B-cell non-Hodgkin's lymphoma (NHL) have, to date, targeted CD19, CD20, or CD22 antigens that are expressed on malignant lymphoid cells as well as on normal B cells (Brentj ens et al., Sci Transl Med 2013; 5(177):177ra38; Haso et al., Blood 2013; 121(7):1165-74; James et al., J Immunol 2008; 180(10):7028-38; Kalos et al., Sci Transl Med 2011; 3(95):95ra73; Kochenderfer et al., J Clin Oncol 2015; 33(6):540-9; Lee et al., Lancet 2015; 385(9967):517-28; Porter et al., Sci Transl 25 Med 2015; 7(303):303ra139; Savoldo et al., J Clin Invest 2011; 121(5):1822-6; Till et al., Blood 2008; 112(6):2261-71; Till et al., Blood 2012; 119(17):3940-50; Coiffier et al., N Engl J Med 2002; 346(4):235-42).

However, adoptive cell therapies are still developing. For example, CAR T cell therapies targeting CD19 in B cell malignancies destroy not only cancerous B cells, but also normal B cells. Reduced or absent numbers of healthy B cells, a condition known as B cell aplasia, may compromise the patient's ability to produce antibodies that fight infections. Modulating the specificity and strength of CAR T immune responses poses another challenge. In an exemplary and tragic case of “on-target off-tumor” toxicity, a patient with metastatic colon cancer died after receiving T cells expressing a chimeric antigen receptor specific for ERBB2 (highly expressed in colon cancer) when the administered cells localized to the lung and triggered a CRS (cytokine release syndrome) event against low levels of ERBB2 in the healthy lung tissue. See, e.g., Morgan et al., Mol. Ther. 18(4):843-851 (2010).

### BRIEF DESCRIPTION OF THE DRAWINGS

FIG. 1 shows schematic diagrams of (top left) an exemplary expression construct encoding an anti-CD19 chimeric antigen receptor (CAR) having a Strep®-Tag II (SEQ ID NO.: 19) (“STII”) peptide hinge region and further encoding a truncated

EGFR transduction marker; (top right) a model of a host cell expressing the encoded anti-CD19-STII CAR; (bottom left) an exemplary expression construct encoding an anti-STII CAR and a truncated EGFR transduction marker; and (bottom right) a model of a host cell expressing the encoded anti-STII CAR.

FIG. 2 shows schematic diagrams of exemplary anti-STII CAR designs. Left: anti-STII CAR with an intermediate-length spacer (IgG4 CH3). Middle: anti-STII CAR with a long spacer (IgG4/2NQ CH2-CH3). Right: descriptions of exemplary anti-STII CARs generated with intermediate or long spacers and scFv binding domains (“5G2” or “3E8”) in the VH-VL or VL-VH orientations.

FIG. 3 shows expression of the constructs depicted in FIG. 2 in primary PBMCs. (A, upper left-hand corner) Untransduced PBMCs. (B, lower left-hand corner) PBMCs transduced with an anti-CD19-STII CAR expression construct. (C, lower right-hand corner) PBMCs transduced with an anti-STII CAR expression construct. Transduced cells were detected in flow cytometry experiments using a biotinylated anti-EGFR monoclonal antibody and streptavidin-PE on day 4 following γ-retroviral transduction of the cells. Cells were pre-gated on living lymphocytes. Numbers indicate the percentage of cells detected.

FIGS. 4A and 4B provide data from flow cytometry experiments showing expression data from (A) untransduced primary PBMCs and (B) primary PBMCs that were transduced to express a high affinity anti-STII CAR of the present disclosure. Transduced cells were detected in flow cytometry experiments using a biotinylated anti-EGFR monoclonal antibody and streptavidin-PE on day 4 following y-retroviral transduction. Cells were pre-gated on living lymphocytes. Numbers indicate the percentage of cells detected.

FIGS. 5A and 5B show specificity and reactivity of exemplary anti-STII CAR T cells according to the present disclosure. (A) IFN-γ production (ng/mL) by human T cells transduced with anti-STII CARs as indicated in the figure legend. X-axis, left to right: negative control (anti-CD19-Hi CAR T cells); anti-CD19 CAR T cells expressing 1, 2, or 3 STII; T cells activated with PMA/IONO (positive control). (B) FACS data showing proliferation of carboxyfluorescein succinimidyl ester (CFSE)-labeled anti-STII CAR T cells following stimulation with either anti-CD19-Hi CAR T cells or medium (top row), or with either anti-CD19-1STII CAR T cells or medium (bottom row).

FIGS. 6A-6C provide data from cytotoxicity assays in which effector T cells expressing the indicated anti-STII CAR constructs were incubated in triplicates with 1×10³ Cr⁵¹-labeled target T cells expressing (A) anti-CD19-Hi CAR T cells; (B) anti-CD19-1STII CART cells; or (C) anti-CD19-3STII CART cells for 4 h at the indicated effector:target ratios (x-axes). Specific lysis was calculated using a standard formula based on chromium-release detection. Data represents means±SD for triplicates.

FIG. 7 shows data from a cytotoxicity assay in which the killing activity of anti-CD19-STII CAR T cells and anti-STII CAR T cells was determined. Circle: co-culture of effector anti-CD19-STII CAR T cells with target CD19⁺K562 cells; square: anti-Strep Tag II CART cells in co-culture with target CD19-1STII CART cells; triangle: co-culture of effector anti-STII CAR T cells with untransduced T cells; diamond: co-culture of effector anti-CD19-STII CAR T cells with target unmodified K562 cells. Y-axis: % specific lysis of the target cells. X-axis: effector:target ratios.

FIG. 8 shows data from a cytotoxicity assay in which effector anti-STII CAR T cells were incubated with target HEK293 cells expressing an anti-CD19-STII CAR. The top three curves (circles, squares, and upward-facing triangles represent data points) indicate killing capacity of anti-STII CARs at the indicated effector:target ratios. The bottom curve (downward-facing triangles) is from a negative control using untransduced cells.

FIG. 9 shows schematic diagrams of anti-STII CAR constructs with murine transmembrane and signaling domains and with either a murine IgG1 CH3 spacer (left) or a Myc-tag spacer (right).

FIGS. 10A and 10B show cytokine production by murine T cells expressing the anti-STII CAR constructs illustrated in FIG. 9 following exposure to target cells. (A) Y axis: IFN-γ production (ng/mL) by murine T cells transduced with anti-STII CARs as indicated in the figure legend. X-axis, from left to right: negative control (murine anti-CD19-Hi CAR T cells); murine anti-CD19-STII CAR T cells with or without truncated EGFR transduction marker; murine T cells activated with PMA/IONO (positive control); medium. (B) Y axis: IL-2 production (ng/mL) by the anti-STII CAR T cells. X-axis, left to right: negative control (murine anti-CD19-Hi CAR T cells); murine anti-CD19-STII CAR T cells with or without truncated EGFR transduction marker; murine T cells activated with PMA/IONO (positive control); medium.

FIGS. 11A-11G show CAR expression and in vivo cytolytic activity of murine anti-STII CAR T cells. (A) Flow cytometry data showing surface expression of anti-STII CARs (indicated at left) in murine T cells. (B) Diagram of an experimental treatment scheme examining the effects of anti-STII CAR T cell therapy in mice with B cell aplasia following administration of anti-CD19-1STII CART cells (1 STII tag) and irradiation. (C) Flow cytometry data showing cell counts (% in PBMC) of target (anti-CD19-1STII CAR T; black circle) and effector (anti-STII CAR T; open circle) cells following transfusion with Group 1 anti-STII CAR T cells according to the treatment scheme shown in (B). (D) Flow cytometry data showing the frequency of B cells (CD19⁺CD45.1⁻); anti-CD19-1STII CART cells (CD45.1⁺EGFR⁺STII⁺); and anti-STII CART cells (CD45.1⁺EGFR⁺Myc⁺) in PBMC of control or Group 1 mice at Day +3 and Day +42 post-infusion of the anti-STII CAR T cells. (E) Flow cytometry data showing cell counts (% in PBMC) of target (anti-CD19-1STII CAR T) and effector (anti-STII CAR T) cells following transfusion with Group 2 anti-STII CAR T cells (see (B)). (F) Data from flow cytometry experiments showing the frequency of B cells (CD19⁺CD45.1), anti-CD19-1STII CART cells (CD45.1⁺EGFR⁺STII⁺), and anti-STII CART cells (CD45.1⁺EGFR⁺) Myc⁺) in PBMC of control and Group 2 mice at Day +3 (top six panels) and Day +42 (bottom six panels) post-infusion of the anti-CD19-STII CAR T cells. (G) Summary of flow cytometry data showing B cell frequency in PBMC in treated mice versus healthy mice.

FIG. 12A shows a diagram of an experimental treatment scheme examining the effects of anti-STII CAR T cell therapy in mice with B cell aplasia following administration of anti-CD19-3STII CART cells (3 STII tags) and irradiation. FIG. 12B provides data from flow cytometry experiments showing counts of anti-CD19-3 STII CART cells (left) and sorted anti-STII CAR T cells (right) used in the treatment.

FIGS. 13A-13I show B cell depletion in mice that received treatment according to the schedule shown in FIG. 12(A), as measured prior to transfusion with anti-STII CAR T cells. (A-H) data from flow cytometry experiments: (A) forward scatter (FS) log vs. side scatter (SS) log plot for lineage-marked PBMCs; gating for live lymphocytes; (B) scatter plot for TX Red (Y-axis) vs. phycoerythrin-conjugated anti-CD19 antibody (CD19-PE) (X-axis); gating for live cells; (C) SS log vs. CD19PE; (D) histogram summarizing cell counts from the experiment shown in FIG. 13(C); CD19⁺ fraction shown in scatter plot (E) and histogram (F), with CD19-depleted fraction (G, H). (I) B cell depletion in PBMCs, as determined using anti-PE magnetic beads following staining with CD19PE.

FIG. 14 provides data from flow cytometry experiments measuring B cell counts in PBMCs from mice receiving the treatment shown in FIG. 12(A). Top row (“pos”): cells from mice that did not receive radiation or anti-CD19-3STII CAR T cells. Middle row (“sample”): cells from mice that received radiation and anti-CD19-3STII CAR T cells, followed by anti-STII CAR T cells. Bottom row (“neg”): cells from mice that received radiation and anti-CD19-3STII CART cells, but did not receive anti-STII CART cells. Y-axes: antibody against Natural Killer cell surface antigen 1.1 (NK1.1). X-axes: CD19⁺ cells (staining with anti-CD19 antibody).

FIG. 15A provides data from flow cytometry experiments showing cell counts (% in blood) of anti-CD19-3STII CART (triangles); OT-1 CD45.1/2⁺ anti-STII CAR T (squares); and CD90.1⁺ CAR T cells (triangles) over the course of the treatment schedule shown in FIG. 12A. FIG. 15B provides data from flow cytometry experiments showing endogenous B cell counts (% in blood) over the course of the treatment scheme shown in FIG. 12A. “Pos”: cells from mice that did not receive radiation or anti-CD19-3STII CART cells. “Sample”: cells from mice that received radiation and anti-CD19-3STII CAR T cells, followed by anti-STII CAR T cells. “Neg”: cells from mice that received radiation and anti-CD19-3STII CAR T cells, but did not receive anti-STII CAR T cells. Gray shading=window of B cell aplasia.

FIGS. 16A-16D show data from flow cytometry experiments measuring cell counts of B cells (stained with anti-CD19 antibody), anti-CD19-3STII CAR T cells, and anti-STII CAR T cells (stained with anti-EGFRt antibody) upon conclusion of the treatment schedule shown in FIG. 15A. Samples were taken from: (A) blood; (B) bone marrow; (C) lymph node; and (D) spleen.

FIGS. 17A-17C show schematic diagrams of exemplary expression constructs of the present disclosure. (A) Expression construct encoding an anti-CD19 CAR having a 3STII hinge region and further encoding a truncated EGFR transduction marker, wherein the EGFRt-encoding portion is separated from the CAR-encoding portion by a polynucleotide encoding a self-cleaving P2A polypeptide (“m19-3STII-28z_E”). (B) Expression construct encoding an anti-CD19 CAR with a CD8 hinge, CD8 transmembrane portion, and CD28-4-1BB-z signaling domains, and further encoding an EGFRt transduction marker fused to a 3STII peptide, wherein the EGFRt-3STII-encoding portion is separated from the CAR-encoding portion by a polynucleotide encoding a self-cleaving P2A polypeptide. (“m19-28z-E-3STII”). (C) Expression construct encoding an anti-STII CAR and a truncated EGFR transduction marker, with the CAR- and marker-encoding portions separated by a polynucleotide encoding a self-cleaving P2A polypeptide. FIGS. 17D-17F provide representative data from flow cytometry experiments showing expression of the indicated constructs by transduced cells (at left), with schematic diagrams of the cells at right.

FIG. 18A shows a diagram of an experimental treatment scheme wherein sublethally irradiated (6Gy) C57/BL6 mice were administered 2×10⁶ murine CD90.1^{+/-} T cells expressing either (1) m19-3STII-28z E or (2) m19-28z E-3STII at Day 40.

FIG. 18B provides data from flow cytometry experiments showing cell surface expression of (1) m19-3STII-28z E or (2) m19-28z_E-3STII. Cells were stained using anti-ST-allophycocyanin (Y-axes) and anti-EGFRt (X-axes).

FIG. 19 provides data from flow cytometry experiments showing B cell depletion in CD90.1^{+/-}C57/BL6 mice receiving: m19-3STII-28z E CART cells (left panels) (n=2); T cells expressing an anti-CD19 CAR without an STII peptide (middle panel); or m19-28z E-3STII (right panels) (n=2). B cells were stained using an anti-CD19 antibody.

FIG. 20A shows a diagram of an experimental treatment scheme wherein sublethally irradiated (6Gy) C57/BL6 mice were administered 2×10⁶murine CD90.1^{+/-} T cells expressing either (1) m19-3STII-28z E or (2) m19-28z E-3STII at Day 0, followed by transfusion with 2.5×10⁶ CD45.1^{+/-} anti-STII CART cells at Day +40.

FIG. 20B provides data from flow cytometry experiments showing cell surface expression of (1) m19-3STII-28z-E and (2) m19-28z-E-3STII. (3) Histogram showing expression of anti-STII CAR construct in transduced T cells.

FIGS. 21A(i)-(ii) and 21B(i)-(ii) show data from flow cytometry experiments conducted 6 days after injection of anti-STII CAR T cells according to the treatment scheme shown in FIG. 20(A). (A) Scatter plots from mice injected with T cells expressing m19-3STII-28z_E. N=2 (i, ii). Gating for B cells. At right, (a) and (b) show expression of the indicated constructs in the transduced T cells. (B) Scatter plots from mice injected with T cells expressing m19-28z E3STII. N=2 (i, ii). Gating for B cells. At left, (a) and (b) show expression of the constructs in the transduced T cells.

FIGS. 22A(i)-(ii) and 22B(i)-(ii) show data from flow cytometry experiments conducted 30 days after injection of anti-STII CAR T cells according to the treatment scheme shown in FIG. 20A. (A) Scatter plots from mice injected with T cells expressing m19-3STII-28z_E. N=2 (i, ii). Gating for B cells. At right, (a) and (b) show expression of the constructs in the transduced T cells. (B) Scatter plots from mice injected with T cells expressing m19-28z_E3STII. N=2 (i, ii). Gating for B cells. At left, (a) and (b) show expression of the constructs in the transduced T cells.

FIGS. 23A and 23B show data from flow cytometry experiments measuring counts of B cells (large panels, staining with anti-CD19 antibody), anti-CD19-3STII CAR T cells, and anti-STII CAR T cells (small panels, staining with anti-EGFRt antibody) upon conclusion of the treatment scheme shown in FIG. 20(A). Samples were taken from: (A) (top) blood; (bottom) bonemarrow; (B) (top) lymph node; and (bottom) spleen. Expression of the CAR constructs by transduced and transferred T cells was analyzed as shown in FIGS. 22A(i)(a-b), (ii)(a-b) and 22B (i)(a-b), (ii)(a-b).

### DETAILED DESCRIPTION

The present disclosure provides tag-specific fusion proteins for selectively detecting molecules containing a Strep-tag or cells containing a Strep-tag. The tag-specific fusion proteins can be used for monitoring and/or modulating the activity of immunotherapy cells expressing a tagged cell surface molecule, such as a CAR or a marker containing a Strep-tag. Exemplary fusion proteins (or cells expressing such fusion proteins on their cell surface) of this disclosure for detecting tagged molecules or tagged cells can comprise (a) an extracellular component comprising a binding domain that specifically binds to a strep-tag peptide (as defined herein; e.g., a peptide comprising or consisting of the amino acid sequence WSHPQFEK (SEQ ID NO:19));

(b) an intracellular component comprising an effector domain or a functional portion thereof; and (c) a transmembrane domain connecting the extracellular and intracellular components.

In certain embodiments, the instant disclosure provides fusion proteins (or cells expressing such fusion proteins on their cell surface) that can detect or ablate target cells that contain: a first polynucleotide encoding a cell surface receptor that includes (a) an extracellular component comprising a binding domain that specifically binds a target antigen, (b) an intracellular component comprising an effector domain or a functional portion thereof, and (c) a transmembrane component connecting the extracellular component and the intracellular component; a second polynucleotide encoding a tagged marker and comprising a polynucleotide encoding the marker containing a tag peptide, wherein the encoded tag peptide comprises a strep-tag peptide optionally comprising or consisting of the amino acid sequence shown in SEQ ID NO: 19; and a third polynucleotide encoding a self-cleaving polypeptide disposed between the first polynucleotide encoding the cell surface receptor and the second polynucleotide encoding the tagged marker. In some embodiments, a presently disclosed fusion protein (or a cell expressing the same on its cell surface) can detect or ablate a target cell that expresses a fusion protein comprising a strep-tag peptide (e.g., comprising or consisting of the amino acid sequence shown in SEQ ID NO:19). In certain embodiments, a fusion protein that comprises a strep-tag peptide comprises a marker, a cell surface receptor, or both, as discussed further herein.

Compositions of the present disclosure are useful in methods of, for example, modulating cell therapies comprising tagged cells, such as tagged cells used in cellular immunotherapy, grafts and transplants. For example, immunotherapy cells expressing heterologous molecules, such as a chimeric antigen receptor (CAR) or T cell receptor (TCR), may have little effect or may lead to one or more adverse events when administered. The present disclosure provides reagents for modulating (e.g., neutralizing, killing, activating, stimulating, or otherwise modulating) immunotherapy cells. The compositions and methods described herein will in certain embodiments have utility for selectively modulating (e.g., killing or activating, as desired) tagged immunotherapy cells, such as tagged CAR T cells or CAR T cells comprising a tagged marker.

Prior to setting forth this disclosure in more detail, it may be helpful to an understanding thereof to provide definitions of certain terms to be used herein. Additional definitions are set forth throughout this disclosure.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, is to be understood to include any integer within the recited range, unless otherwise indicated. As used herein, the term “about” means±20% of the indicated range, value, or structure, unless otherwise indicated. It should be understood that the terms “a” and “an” as used herein refer to “one or more” of the enumerated components. The use of the alternative (e.g., “or”) should be understood to mean either one, both, or any combination of the alternatives. As used herein, the terms “include,” “have,” and “comprise” are used synonymously, which terms and variants thereof are intended to be construed as non-limiting.

“Optional” or “optionally” means that the subsequently described element, component, event, or circumstance may or may not occur, and that the description includes instances in which the element, component, event, or circumstance occurs and instances in which they do not.

In addition, it should be understood that the individual constructs, or groups of constructs, derived from the various combinations of the structures and subunits described herein, are disclosed by the present application to the same extent as if each construct or group of constructs was set forth individually. Thus, selection of particular structures or particular subunits is within the scope of the present disclosure.

The term “consisting essentially of” is not equivalent to “comprising” and refers to the specified materials or steps of a claim, or to those that do not materially affect the basic characteristics of a claimed subject matter. For example, a protein domain, region, or module (e.g., a binding domain, hinge region, or linker) or a protein (which may have one or more domains, regions, or modules) “consists essentially of” a particular amino acid sequence when the amino acid sequence of a domain, region, module, or protein includes extensions, deletions, mutations, or a combination thereof (e.g., amino acids at the amino- or carboxy-terminus or between domains) that, in combination, contribute to at most 20% (e.g., at most 15%, 10%, 8%, 6%, 5%, 4%, 3%, 2% or 1%) of the length of a domain, region, module, or protein and do not substantially affect (i.e., do not reduce the activity by more than 50%, such as no more than 40%, 30%, 25%, 20%, 15%, 10%, 5%, or 1%) the activity of the domain(s), region(s), module(s), or protein (e.g., the target binding affinity of a binding protein).

As used herein, “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally occurring amino acid, i.e., an a-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refer to chemical compounds that have a structure that is different from the general chemical structure of an amino acid, but that function in a manner similar to a naturally occurring amino acid.

As used herein, “mutation” refers to a change in the sequence of a nucleic acid molecule or polypeptide molecule as compared to a reference or wild-type nucleic acid molecule or polypeptide molecule, respectively. A mutation can result in several different types of change in sequence, including substitution, insertion or deletion of nucleotide(s) or amino acid(s).

A “conservative substitution” refers to amino acid substitutions that do not significantly affect or alter binding characteristics of a particular protein. Generally, conservative substitutions are ones in which a substituted amino acid residue is replaced with an amino acid residue having a similar side chain. Conservative substitutions include a substitution found in one of the following groups: Group 1: Alanine (Ala or A), Glycine (Gly or G), Serine (Ser or S), Threonine (Thr or T); Group 2: Aspartic acid (Asp or D), Glutamic acid (Glu or Z); Group 3: Asparagine (Asn or N), Glutamine (Gln or Q); Group 4: Arginine (Arg or R), Lysine (Lys or K), Histidine (His or H); Group 5: Isoleucine (Ile or I), Leucine (Leu or L), Methionine (Met or M), Valine (Val or V); and Group 6: Phenylalanine (Phe or F), Tyrosine (Tyr or Y), Tryptophan (Trp or W). Additionally or alternatively, amino acids can be grouped into conservative substitution groups by similar function, chemical structure, or composition (e.g., acidic, basic, aliphatic, aromatic, or sulfur-containing). For example, an aliphatic grouping may include, for purposes of substitution, Gly, Ala, Val, Leu, and Ile. Other conservative substitutions groups include: sulfur-containing: Met and Cysteine (Cys or C); acidic: Asp, Glu, Asn, and Gln; small aliphatic, nonpolar or slightly polar residues: Ala, Ser, Thr, Pro, and Gly; polar, negatively charged residues and their amides: Asp, Asn, Glu, and Gln; polar, positively charged residues: His, Arg, and Lys; large aliphatic, nonpolar residues: Met, Leu, Ile, Val, and Cys; and large aromatic residues: Phe, Tyr, and Trp. Additional information can be found in Creighton (1984) Proteins, W. H. Freeman and Company.

As used herein, “protein” or “polypeptide” refers to a polymer of amino acid residues. Proteins apply to naturally occurring amino acid polymers, as well as to amino acid polymers in which one or more amino acid residue is an artificial chemical mimetic of a corresponding naturally occurring amino acid and non-naturally occurring amino acid polymers.

As used herein, “fusion protein” refers to a protein that, in a single chain, has at least two distinct domains, wherein the domains are not naturally found together in a protein. A polynucleotide encoding a fusion protein may be constructed using PCR, recombinantly engineered, or the like, or such fusion proteins can be synthesized. A fusion protein may further contain other components, such as a tag, a linker, or a transduction marker. In certain embodiments, a fusion protein expressed or produced by a host cell (e.g., a T cell) locates to the cell surface, where the fusion protein is anchored to the cell membrane (e.g., via a transmembrane domain) and comprises an extracellular portion (e.g., containing a binding domain) and an intracellular portion (e.g., containing a signaling domain, effector domain, co-stimulatory domain or combinations thereof).

“Nucleic acid molecule” or “polynucleotide” refers to a polymeric compound including covalently linked nucleotides, which can be made up of natural subunits (e.g., purine or pyrimidine bases) or non-natural subunits (e.g., morpholine ring). Purine bases include adenine, guanine, hypoxanthine, and xanthine, and pyrimidine bases include uracil, thymine, and cytosine. Nucleic acid molecules include polyribonucleic acid (RNA), polydeoxyribonucleic acid (DNA), which includes cDNA, genomic DNA, and synthetic DNA, either of which may be single or double-stranded. If single-stranded, the nucleic acid molecule may be the coding strand or non-coding (anti-sense strand). A nucleic acid molecule encoding an amino acid sequence includes all nucleotide sequences that encode the same amino acid sequence. Some versions of the nucleotide sequences may also include intron(s) to the extent that the intron(s) would be removed through co- or post-transcriptional mechanisms. In other words, different nucleotide sequences may encode the same amino acid sequence as the result of the redundancy or degeneracy of the genetic code, or by splicing.

Variants of nucleic acid molecules of this disclosure are also contemplated. Variant nucleic acid molecules are at least 70%, 75%, 80%, 85%, 90%, and are preferably 95%, 96%, 97%, 98%, 99%, or 99.9% identical a nucleic acid molecule of a defined or reference polynucleotide as described herein, or that hybridize to a polynucleotide under stringent hybridization conditions of 0.015M sodium chloride, 0.0015M sodium citrate at about 65-68° C. or 0.015M sodium chloride, 0.0015M sodium citrate, and 50% formamide at about 42° C. Nucleic acid molecule variants retain the capacity to encode a fusion protein or a binding domain thereof having a functionality described herein, such as specifically binding a target molecule. “Percent sequence identity” refers to a relationship between two or more sequences, as determined by comparing the sequences. Preferred methods to determine sequence identity are designed to give the best match between the sequences being compared. For example, the sequences are aligned for optimal comparison purposes (e.g., gaps can be introduced in one or both of a first and a second amino acid or nucleic acid sequence for optimal alignment). Further, non-homologous sequences may be disregarded for comparison purposes. The percent sequence identity referenced herein is calculated over the length of the reference sequence, unless indicated otherwise. Methods to determine sequence identity and similarity can be found in publicly available computer programs. Sequence alignments and percent identity calculations may be performed using a BLAST program (e.g., BLAST 2.0, BLASTP, BLASTN, or BLASTX). The mathematical algorithm used in the BLAST programs can be found in Altschul et al., Nucleic Acids Res. 25:3389-3402, 1997. Within the context of this disclosure, it will be understood that where sequence analysis software is used for analysis, the results of the analysis are based on the “default values” of the program referenced. “Default values” mean any set of values or parameters which originally load with the software when first initialized.

The term “isolated” means that the material is removed from its original environment (e.g., the natural environment if it is naturally occurring). For example, a naturally occurring nucleic acid or polypeptide present in a living animal is not isolated, but the same nucleic acid or polypeptide, separated from some or all of the co-existing materials in the natural system, is isolated. Such nucleic acid could be part of a vector and/or such nucleic acid or polypeptide could be part of a composition (e.g., a cell lysate), and still be isolated in that such vector or composition is not part of the natural environment for the nucleic acid or polypeptide. The term “gene” means the segment of DNA involved in producing a polypeptide chain; it includes regions preceding and following the coding region (“leader and trailer”) as well as intervening sequences (introns) between individual coding segments (exons).

A “functional variant” refers to a polypeptide or polynucleotide that is structurally similar or substantially structurally similar to a parent or reference compound of this disclosure, but differs slightly in composition (e.g., one base, atom or functional group is different, added, or removed), such that the polypeptide or encoded polypeptide is capable of performing at least one function of the encoded parent polypeptide with at least 50% efficiency, preferably at least 55%, 60%, 70%, 75%, 80%, 85%, 90%, 95%, 96%, 97%, 98%, 99%, 99.9%, or 100% level of activity of the parent polypeptide. In other words, a functional variant of a polypeptide or encoded polypeptide of this disclosure has “similar binding,” “similar affinity” or “similar activity” when the functional variant displays no more than a 50% reduction in performance in a selected assay as compared to the parent or reference polypeptide, such as an assay for measuring binding affinity (e.g., Biacore® or tetramer staining measuring an association (Kₐ) or a dissociation (K_{D}) constant). As used herein, a “functional portion” or “functional fragment” refers to a polypeptide or polynucleotide that comprises only a domain, portion or fragment of a parent or reference compound, and the polypeptide or encoded polypeptide retains at least 50% activity associated with the domain, portion or fragment of the parent or reference compound, preferably at least 55%, 60%, 70%, 75%, 80%, 85%, 90%, 95%, 96%, 97%, 98%, 99%, 99.9%, or 100% level of activity of the parent polypeptide, or provides a biological benefit (e.g., effector function). A “functional portion” or “functional fragment” of a polypeptide or encoded polypeptide of this disclosure has “similar binding” or “similar activity” when the functional portion or fragment displays no more than a 50% reduction in performance in a selected assay as compared to the parent or reference polypeptide (preferably no more than 20% or 10%, or no more than a log difference as compared to the parent or reference with regard to affinity), such as an assay for measuring binding affinity or measuring effector function (e.g., cytokine release).

As used herein, “heterologous” or “non-endogenous” or “exogenous” refers to any gene, protein, compound, nucleic acid molecule, or activity that is not native to a host cell or a subject, or any gene, protein, compound, nucleic acid molecule, or activity native to a host cell or a subject that has been altered. Heterologous, non-endogenous, or exogenous includes genes, proteins, compounds, or nucleic acid molecules that have been mutated or otherwise altered such that the structure, activity, or both is different as between the native and altered genes, proteins, compounds, or nucleic acid molecules. In certain embodiments, heterologous, non-endogenous, or exogenous genes, proteins, or nucleic acid molecules (e.g., receptors, ligands, etc.) may not be endogenous to a host cell or a subject, but instead nucleic acids encoding such genes, proteins, or nucleic acid molecules may have been added to a host cell by conjugation, transformation, transfection, electroporation, or the like, wherein the added nucleic acid molecule may integrate into a host cell genome or can exist as extra-chromosomal genetic material (e.g., as a plasmid or other self-replicating vector). The term “homologous” or “homolog” refers to a gene, protein, compound, nucleic acid molecule, or activity found in or derived from a host cell, species, or strain. For example, a heterologous or exogenous polynucleotide or gene encoding a polypeptide may be homologous to a native polynucleotide or gene and encode a homologous polypeptide or activity, but the polynucleotide or polypeptide may have an altered structure, sequence, expression level, or any combination thereof. A non-endogenous polynucleotide or gene, as well as the encoded polypeptide or activity, may be from the same species, a different species, or a combination thereof.

As used herein, the term “endogenous” or “native” refers to a polynucleotide, gene, protein, compound, molecule, or activity that is normally present in a host cell or a subject.

The term “expression”, as used herein, refers to the process by which a polypeptide is produced based on the encoding sequence of a nucleic acid molecule, such as a gene. The process may include transcription, post-transcriptional control, post-transcriptional modification, translation, post-translational control, post-translational modification, or any combination thereof. An expressed nucleic acid molecule is typically operably linked to an expression control sequence (e.g., a promoter).

The term “operably linked” refers to the association of two or more nucleic acid molecules on a single nucleic acid fragment so that the function of one is affected by the other. For example, a promoter is operably linked with a coding sequence when it is capable of affecting the expression of that coding sequence (i.e., the coding sequence is under the transcriptional control of the promoter). “Unlinked” means that the associated genetic elements are not closely associated with one another and the function of one does not affect the other.

As used herein, “expression vector” refers to a DNA construct containing a nucleic acid molecule that is operably linked to a suitable control sequence capable of effecting the expression of the nucleic acid molecule in a suitable host. Such control sequences include a promoter to effect transcription, an optional operator sequence to control such transcription, a sequence encoding suitable mRNA ribosome binding sites, and sequences which control termination of transcription and translation. The vector may be a plasmid, a phage particle, a virus, or simply a potential genomic insert. Once transformed into a suitable host, the vector may replicate and function independently of the host genome, or may, in some instances, integrate into the genome itself In the present specification, “plasmid,” “expression plasmid,” “virus” and “vector” are often used interchangeably.

The term “introduced” in the context of inserting a nucleic acid molecule into a cell, means “transfection”, or “transformation” or “transduction” and includes reference to the incorporation of a nucleic acid molecule into a eukaryotic or prokaryotic cell wherein the nucleic acid molecule may be incorporated into the genome of a cell (e.g., chromosome, plasmid, plastid, or mitochondrial DNA), converted into an autonomous replicon, or transiently expressed (e.g., transfected mRNA). As used herein, the term “engineered,” “recombinant” or “non-natural” refers to an organism, microorganism, cell, nucleic acid molecule, or vector that includes at least one genetic alteration or has been modified by introduction of an exogenous nucleic acid molecule, wherein such alterations or modifications are introduced by genetic engineering (i.e., human intervention). Genetic alterations include, for example, modifications introducing expressible nucleic acid molecules encoding proteins, fusion proteins or enzymes, or other nucleic acid molecule additions, deletions, substitutions or other functional disruption of a cell's genetic material. Additional modifications include, for example, non-coding regulatory regions in which the modifications alter expression of a polynucleotide, gene or operon.

As described herein, more than one heterologous nucleic acid molecule can be introduced into a host cell as separate nucleic acid molecules, as a plurality of individually controlled genes, as a polycistronic nucleic acid molecule, as a single nucleic acid molecule encoding a fusion protein, or any combination thereof. When two or more heterologous nucleic acid molecules are introduced into a host cell, it is understood that the two or more heterologous nucleic acid molecules can be introduced as a single nucleic acid molecule (e.g., on a single vector), on separate vectors, integrated into the host chromosome at a single site or multiple sites, or any combination thereof. The number of referenced heterologous nucleic acid molecules or protein activities refers to the number of encoding nucleic acid molecules or the number of protein activities, not the number of separate nucleic acid molecules introduced into a host cell.

The term “construct” refers to any polynucleotide that contains a recombinant nucleic acid molecule. A construct may be present in a vector (e.g., a bacterial vector, a viral vector) or may be integrated into a genome. A “vector” is a nucleic acid molecule that is capable of transporting another nucleic acid molecule. Vectors may be, for example, plasmids, cosmids, viruses, a RNA vector or a linear or circular DNA or RNA molecule that may include chromosomal, non-chromosomal, semi-synthetic or synthetic nucleic acid molecules. Vectors of the present disclosure also include transposon systems (e.g., Sleeping Beauty, see, e.g., Geurts et al., Mol. Ther. 8:108, 2003: Mates et al., Nat. Genet. 41:753, 2009). Exemplary vectors are those capable of autonomous replication (episomal vector), capable of delivering a polynucleotide to a cell genome (e.g., viral vector), or capable of expressing nucleic acid molecules to which they are linked (expression vectors).

As used herein, the term “host” refers to a cell (e.g., T cell) or microorganism targeted for genetic modification with a heterologous nucleic acid molecule to produce a polypeptide of interest (e.g., a fusion protein of the present disclosure). In certain embodiments, a host cell may optionally already possess or be modified to include other genetic modifications that confer desired properties related or unrelated to, e.g., biosynthesis of the heterologous protein (e.g., inclusion of a detectable marker; deleted, altered or truncated endogenous TCR; or increased co-stimulatory factor expression).

As used herein, “enriched” or “depleted” with respect to amounts of cell types in a mixture refers to an increase in the number of the “enriched” type, a decrease in the number of the “depleted” cells, or both, in a mixture of cells resulting from one or more enriching or depleting processes or steps. Thus, depending upon the source of an original population of cells subjected to an enriching process, a mixture or composition may contain 30%, 35%, 40%, 45%, 50%, 55%, 60%, 65%, 70%, 75%, 80%, 85%, 90%, 91%, 92%, 93%, 94%, 95%, 96%, 97%, 98%, or 99% or more (in number or count) of the “enriched” cells. Cells subjected to a depleting process can result in a mixture or composition containing 50%, 45%, 40%, 35%, 30%, 25%, 20%, 15%, 10%, 9%, 8%, 7%, 6%, 5%, 4%, 3%, 2%, or 1% percent or less (in number or count) of the “depleted” cells. In certain embodiments, amounts of a certain cell type in a mixture will be enriched and amounts of a different cell type will be depleted, such as enriching for CD4⁺ cells while depleting CD8⁺ cells, or enriching for CD62L⁺ cells while depleting CD62L⁻ cells, or combinations thereof.

“T cell receptor” (TCR) refers to an immunoglobulin superfamily member (having a variable binding domain, a constant domain, a transmembrane region, and a short cytoplasmic tail; see, e.g., Janeway et al., Immunobiology: The Immune System in Health and Disease, 3^{rd} Ed., Current Biology Publications, p. 4:33, 1997) capable of specifically binding to an antigen peptide bound to a MHC receptor. A TCR can be found on the surface of a cell or in soluble form and generally is comprised of a heterodimer having α and β chains (also known as TCRα and TCRβ, respectively), or γ and δ chains (also known as TCRγ and TCRδ, respectively). Like immunoglobulins, the extracellular portion of TCR chains (e.g., α-chain, β-chain) contain two immunoglobulin domains, a variable domain (e.g., α-chain variable domain or V_{α}, β-chain variable domain or V_{β}; typically amino acids 1 to 116 based on Kabat numbering (Kabat et al., “Sequences of Proteins of Immunological Interest,” US Dept. Health and Human Services, Public Health Service National Institutes of Health, 1991, 5^{th} ed.) at the N-terminus, and one constant domain (e.g., a-chain constant domain or Cₐ, typically amino acids 117 to 259 based on Kabat, β-chain constant domain or C_{α}, typically amino acids 117 to 295 based on Kabat) adjacent to the cell membrane. Also, like immunoglobulins, the variable domains contain complementary determining regions (CDRs) separated by framework regions (FRs) (see, e.g., Jores et al., Proc. Nat'l Acad. Sci. U.S.A. 87:9138, 1990; Chothia et al., EMBO J. 7:3745, 1988; see also Lefranc et al., Dev. Comp. Immunol. 27:55, 2003). In certain embodiments, a TCR is found on the surface of T cells (or T lymphocytes) and associates with the CD3 complex. The source of a TCR as used in the present disclosure may be from various animal species, such as a human, mouse, rat, rabbit or other mammal.

“CD3” is known in the art as a multi-protein complex of six chains (see, Abbas and Lichtman, 2003; Janeway et al., p. 172 and 178, 1999). In mammals, the complex comprises a CD3γ chain, a CD3δ chain, two CD3ε chains, and a homodimer of CD3ζ chains. The CD3γ, CD3δ, and CD3ε chains are highly related cell surface proteins of the immunoglobulin superfamily containing a single immunoglobulin domain. The transmembrane regions of the CD3γ, CD3δ, and CD3ε chains are negatively charged, which is a characteristic that allows these chains to associate with the positively charged T cell receptor chains. The intracellular tails of the CD3γ, CD3δ, and CD3ζchains each contain a single conserved motif known as an immunoreceptor tyrosine-based activation motif or ITAM, whereas each CD3 chain has three ITAMs. Without wishing to be bound by theory, it is believed that the ITAMs are important for the signaling capacity of a TCR complex. CD3 as used in the present disclosure may be from various animal species, including human, mouse, rat, or other mammals.

“Major histocompatibility complex molecules” (MHC molecules) refer to glycoproteins that deliver peptide antigens to a cell surface. MHC class I molecules are heterodimers consisting of a membrane spanning a chain (with three α domains) and a non-covalently associated β2 microglobulin. MHC class II molecules are composed of two transmembrane glycoproteins, α and β, both of which span the membrane. Each chain has two domains. MHC class I molecules deliver peptides originating in the cytosol to the cell surface, where a peptide:MHC complex is recognized by CD8⁺ T cells. MHC class II molecules deliver peptides originating in the vesicular system to the cell surface, where they are recognized by CD4⁺ T cells. An MHC molecule may be from various animal species, including human, mouse, rat, cat, dog, goat, horse, or other mammals.

“CD4” refers to an immunoglobulin co-receptor glycoprotein that assists the TCR in communicating with antigen-presenting cells (see, Campbell & Reece, Biology 909 (Benjamin Cummings, Sixth Ed., 2002); UniProtKB P01730). CD4 is found on the surface of immune cells such as T helper cells, monocytes, macrophages, and dendritic cells, and includes four immunoglobulin domains (D1 to D4) that are expressed at the cell surface. During antigen presentation, CD4 is recruited, along with the TCR complex, to bind to different regions of the MHCII molecule (CD4 binds MHCII (32, while the TCR complex binds MHCII α1/β1). Without wishing to be bound by theory, it is believed that close proximity to the TCR complex allows CD4-associated kinase molecules to phosphorylate the immunoreceptor tyrosine activation motifs (ITAMs) present on the cytoplasmic domains of CD3. This activity is thought to amplify the signal generated by the activated TCR in order to produce various types of T helper cells.

As used herein, the term “CD8 co-receptor” or “CD8” means the cell surface glycoprotein CD8, either as an alpha-alpha homodimer or an alpha-beta heterodimer. The CD8 co-receptor assists in the function of cytotoxic T cells (CD8⁺) and functions through signaling via its cytoplasmic tyrosine phosphorylation pathway (Gao and Jakobsen, Immunol. Today 21:630-636, 2000; Cole and Gao, Cell. Mol. Immunol. 1:81-88, 2004). In humans, there are five (5) different CD8 beta chains (see UniProtKB identifier P10966) and a single CD8 alpha chain (see UniProtKB identifier P01732).

“Chimeric antigen receptor” (CAR) refers to a fusion protein of the present disclosure engineered to contain two or more naturally occurring amino acid sequences linked together in a way that does not occur naturally or does not occur naturally in a host cell, which fusion protein can function as a receptor when present on a surface of a cell. CARs of the present disclosure include an extracellular portion comprising an antigen binding domain (i.e., obtained or derived from an immunoglobulin or immunoglobulin-like molecule, such as a scFv or scTCR derived from an antibody or TCR specific for a cancer antigen, or an antigen-binding domain derived or obtained from a killer immunoreceptor from an NK cell) linked to a transmembrane domain and one or more intracellular signaling domains (optionally containing co-stimulatory domain(s)) (see, e.g., Sadelain et al., Cancer Discov., 3(4):388 (2013); see also Harris and Kranz, Trends Pharmacol. Sci., 37(3):220 (2016); Stone et al., Cancer Immunol. Immunother., 63(11):1163 (2014)). In certain embodiments, a binding protein comprises a CAR comprising an antigen-specific TCR binding domain (see, e.g., Walseng et al., Scientific Reports 7:10713, 2017; the TCR CAR constructs and methods of which are hereby incorporated by reference in their entirety).

The term “variable region” or “variable domain” refers to the domain of a TCR α-chain or β-chain (or γ-chain and δ-chain for γδ TCRs), or of an antibody heavy or light chain, that is involved in binding to antigen. The variable domains of the a-chain and β-chain (Vα and Vβ, respectively) of a native TCR generally have similar structures, with each domain comprising four generally conserved framework regions (FRs) and three CDRs. Variable domains of antibody heavy (V_{H}) and light (V_{L}) chains each also generally comprise four generally conserved framework regions (FRs) and three CDRs.

The terms “complementarity determining region,” and “CDR,” are synonymous with “hypervariable region” or “HVR,” and are known in the art to refer to non-contiguous sequences of amino acids within TCR or antibody variable regions, which confer antigen specificity and/or binding affinity. In general, there are three CDRs in each variable region(i.e., three CDRs in each of the TCRα-chain and β-chain variable regions; 3 CDRs in each of the antibody heavy chain and light chain variable regions). In the case of TCRs, CDR3 is thought to be the main CDR responsible for recognizing processed antigen. CDR1 and CDR2 mainly interact with the MHC. Variable domain sequences can be aligned to a numbering scheme (e.g., Kabat, EU, International Immunogenetics Information System (IMGT) and Aho), which can allow equivalent residue positions to be annotated and for different molecules to be compared using Antigen receptor Numbering And Receptor Classification (ANARCI) software tool (2016, Bioinformatics 15:298-300).

“Antigen” or “Ag” as used herein refers to an immunogenic molecule that provokes an immune response. This immune response may involve antibody production, activation of specific immunologically-competent cells (e.g., T cells), or both. An antigen (immunogenic molecule) may be, for example, a peptide, glycopeptide, polypeptide, glycopolypeptide, polynucleotide, polysaccharide, lipid or the like. It is readily apparent that an antigen can be synthesized, produced recombinantly, or derived from a biological sample. Exemplary biological samples that can contain one or more antigens include tissue samples, tumor samples, cells, biological fluids, or combinations thereof. Antigens can be produced by cells that have been modified or genetically engineered to express an antigen.

The term “epitope” or “antigenic epitope” includes any molecule, structure, amino acid sequence or protein determinant that is recognized and specifically bound by a cognate binding molecule, such as an immunoglobulin, T cell receptor (TCR), chimeric antigen receptor, or other binding molecule, domain or protein. Epitopic determinants generally contain chemically active surface groupings of molecules, such as amino acids or sugar side chains, and can have specific three dimensional structural characteristics, as well as specific charge characteristics.

“Treat” or “treatment” or “ameliorate” refers to medical management of a disease, disorder, or condition of a subject (e.g., a human or non-human mammal, such as a primate, horse, cat, dog, goat, mouse, or rat). In general, an appropriate dose or treatment regimen comprising a host cell expressing a fusion protein of the present disclosure, and optionally an adjuvant, is administered in an amount sufficient to elicit a therapeutic or prophylactic benefit. Therapeutic or prophylactic/preventive benefit includes improved clinical outcome; lessening or alleviation of symptoms associated with a disease (e.g., B cell aplasia); decreased occurrence of symptoms; improved quality of life; longer disease-free status; diminishment of extent of disease; stabilization of disease state; delay of disease progression; remission; survival; prolonged survival; or any combination thereof.

A “therapeutically effective amount” or “effective amount” of a fusion protein or host cell expressing a fusion protein of this disclosure, refers to an amount of fusion proteins or host cells sufficient to result in a therapeutic effect, including improved clinical outcome; lessening or alleviation of symptoms associated with a disease; decreased occurrence of symptoms; improved quality of life; longer disease-free status;

diminishment of extent of disease, stabilization of disease state; delay of disease progression; remission; survival; or prolonged survival in a statistically significant manner. When referring to an individual active ingredient or a cell expressing a single active ingredient, administered alone, a therapeutically effective amount refers to the effects of that ingredient or cell expressing that ingredient alone. When referring to a combination, a therapeutically effective amount refers to the combined amounts of active ingredients or combined adjunctive active ingredient with a cell expressing an active ingredient that results in a therapeutic effect, whether administered serially or simultaneously. A combination may also be a cell expressing more than one active ingredient, such as two different fusion proteins (e.g., CARs) that specifically bind a strep tag peptide (e.g., comprising or consisting of the amino acid sequence shown in SEQ ID NO:19), or a fusion protein of the present disclosure.

The term “pharmaceutically acceptable excipient or carrier” or “physiologically acceptable excipient or carrier” refer to biologically compatible vehicles, e.g., physiological saline, which are described in greater detail herein, that are suitable for administration to a human or other non-human mammalian subject and generally recognized as safe or not causing a serious adverse event.

As used herein, “statistically significant” refers to a p-value of 0.050 or less when calculated using the Student's t-test and indicates that it is unlikely that a particular event or result being measured has arisen by chance.

As used herein, the term “adoptive immune therapy” or “adoptive immunotherapy” refers to administration of naturally occurring or genetically engineered, disease-antigen-specific immune cells (e.g., T cells). Adoptive cellular immunotherapy may be autologous (immune cells are from the recipient), allogeneic (immune cells are from a donor of the same species) or syngeneic (immune cells are from a donor genetically identical to the recipient).

“Targeted ablation,” as used herein, refers to selective killing (e.g., by induced apoptosis, lysis, phagocytosis, complement-dependent cytotoxicity (CDC), or antibody-dependent cell-mediated cytotoxicity (ADCC), or by another mechanism) of target cells (e.g., cells expressing a tag peptide having the amino acid sequence shown in SEQ ID NO:19). As described herein, host cells expressing fusion proteins of the present disclosure selectively (i.e., specifically or preferentially) target cells expressing a tag peptide having the amino acid sequence shown in SEQ ID NO: 19 over other cells, wherein binding to the target cells induces a targeted immune response that ablates the target (i.e., tagged) cells.

In any of the presently disclosed embodiments, a fusion protein or binding domain thereof is capable of specifically binding to a strep-tag peptide. As used herein, the term “strep-tag peptide” refers to a peptide that is capable of specifically binding to streptavidin (which is a tetrameric protein purified from Streptomyces avidinii and is widely used in molecule biology protocols due to its high affinity for biotin) or to streptactin, which is an engineered mutein of streptavidin. Exemplary strep-tag peptides of the instant disclosure compete with biotin for binding to streptavidin or streptactin and include, for example, the original Strep® tag (WRHPQFGG, SEQ ID NO:48); Strep® Tag II (also referred to as “STII” herein, which is an optimized version of the original Strep-Tag® and consists of the amino acid sequence WSHPQFEK (SEQ ID NO:19)); and variants thereof, including those disclosed in, for example, Schmidt and Skerra, Nature Protocols, 2:1528-1535 (200), U.S. Pat. No. 7,981,632; and PCT Publication No. WO 2015/067768, the strep-tag peptides, step-tag-peptide-containing polypeptides, and sequences of the same, are incorporated herein by reference.

### Fusion Proteins

In certain aspects, the present disclosure provides fusion proteins, comprising: (a) an extracellular component comprising a binding domain that specifically binds to a strep-tag peptide; (b) an intracellular component comprising an effector domain or a functional portion thereof; and (c) a transmembrane domain connecting the extracellular and intracellular components.

In certain embodiments, the strep-tag peptide comprises or consists of the amino acid sequence shown in SEQ ID NO:19.

A “binding domain” (also referred to as a “binding region” or “binding moiety”), as used herein, refers to a molecule or portion thereof (e.g., peptide, oligopeptide, polypeptide, protein (e.g., a fusion protein)) that possesses the ability to specifically and non-covalently associate, unite, or combine with a target (e.g., a peptide comprising the amino acid sequence shown in SEQ ID NO: 19). A binding domain includes any naturally occurring, synthetic, semi-synthetic, or recombinantly produced binding partner for a biological molecule, a molecular complex (i.e., complex comprising two or more biological molecules), or other target of interest. Exemplary binding domains include single chain immunoglobulin variable regions (e.g., scTCR, scFv, Fab, TCR variable regions), receptor ectodomains, ligands (e.g., cytokines, chemokines), or synthetic polypeptides selected for their specific ability to bind to a biological molecule, a molecular complex or other target of interest. In certain embodiments, the binding domain is a scFv, scTCR, or ligand. In certain embodiments, the binding domain is chimeric, human, or humanized.

In some embodiments, the binding domain comprises: (a) the heavy chain CDR 1 amino acid sequence shown in any one of SEQ ID NOs: 22, 28, or 34, or a variant of SEQ ID NO: 22, 28, or 34 having 1 to 3 amino acid substitutions and/or deletions; (b) the heavy chain CDR 2 amino acid sequence shown in any one of SEQ ID NOs: 23, 29, or 35, or a variant of SEQ ID NO: 23, 29, or 35 having 1 to 3 amino acid substitutions and/or deletions; and (c) the heavy chain CDR 3 amino acid sequence shown in any one of SEQ ID NOs: 24, 30, or 36, or a variant of SEQ ID NO: 24, 30, or 36 having 1 to 3 amino acid substitutions and/or deletions.

In certain embodiments, the binding domain comprises (a) the light chain CDR 1 amino acid sequence shown in any one of SEQ ID NOs: 25, 31, or 37, or a variant of SEQ ID NO: 25, 31, or 37 having 1 to 3 amino acid substitutions and/or deletions; (b) the light chain CDR 2 amino acid sequence shown in any one of SEQ ID NOs: 26, 32, or 38, or a variant of SEQ ID NO: 26, 32, or 38 having 1 or 2 amino acid substitutions and/or deletions; and (c) the light chain CDR 3 amino acid sequence shown in any one of SEQ ID NOs: 27, 33, or 39, or a variant of SEQ ID NO: 27, 33, or 39 having 1 to 3 amino acid substitutions, and/or deletions.

In any of the presently disclosed embodiments, a binding domain may comprise CDR sequences from 5G2 antibody, 3E8 antibody, 4E2 antibody, 3C9 antibody, or 4C4 antibody.

In some embodiments, the binding domain comprises: (a) the heavy chain CDR1 amino acid sequence shown in SEQ ID NO:28; (b) the heavy chain CDR2 amino acid sequence shown in SEQ ID NO:29; (c) the heavy chain CDR3 acid sequence shown in SEQ ID NO:30; (d) the light chain CDR1 amino acid sequence shown in SEQ

ID NO:31; (e) the light chain CDR2 amino acid sequence shown in SEQ ID NO:32; and (e) the light chain CDR3 acid sequence shown in SEQ ID NO:33.

In other embodiments, the binding domain comprises: (a) the heavy chain CDR1 amino acid sequence shown in SEQ ID NO:22; (b) the heavy chain CDR2 amino acid sequence shown in SEQ ID NO:23; (c) the heavy chain CDR3 acid sequence shown in

SEQ ID NO:24; (d) the light chain CDR1 amino acid sequence shown in SEQ ID NO:25; (e) the light chain CDR2 amino acid sequence shown in SEQ ID NO:26; and (e) the light chain CDR3 acid sequence shown in SEQ ID NO:27. In still other embodiments, the binding domain comprises: (a) the heavy chain CDR1 amino acid sequence shown in SEQ ID NO:34; (b) the heavy chain CDR2 amino acid sequence shown in SEQ ID NO:35; (c) the heavy chain CDR3 acid sequence shown in SEQ ID NO:36; (d) the light chain CDR1 amino acid sequence shown in SEQ ID NO:37; (e) the light chain CDR2 amino acid sequence shown in SEQ ID NO:38; and (e) the light chain CDR3 acid sequence shown in SEQ ID NO:39.

In yet other embodiments, a binding domain of the present disclosure comprises CDRs and, optionally, V_{H} and V_{L} sequences of “C23.21” antibody, as disclosed in PCT Publication No. WO 2015/067768, the CDR, V_{H}, and V_{L} sequences of which are hereby incorporated by reference.

Additional antibodies from which a binding domain of the present disclosure may be obtained or derived include “Anti-Strep-tag II antibody” (ab76949), available commercially from Abcam®; “StrepMAB-Immo,” and “StrepMAB-Classic,” both of which are disclosed in, for example, Schmidt and Skerra, Nature Protocols, 2:1528-1535 (2007), and available commercially from Iba Life Sciences; and Strep-tag Antibody (Qiagen, cat. no. 34850). The CDR, V_{H}, and V_{L} sequences of these antibodies are also incorporated by reference.

In certain embodiments, the binding domain is a scFv comprising a V_{H} domain, a V_{L} domain, and a peptide linker. In particular embodiments, a scFv comprises a V_{H} domain joined to a V_{L} domain by a peptide linker, which can be in a V_{H}-linker-V_{L} orientation or in a V_{L}-linker-V_{H} orientation. In some embodiments, a scFv comprises a V_{H} domain, a V_{L} domain, and a peptide linker, wherein the a V_{H} and V_{L} domains are based on the V_{H} and V_{L} domains of 3E8 antibody, 5G2 antibody, 4E2 antibody, 3C9 antibody, or 4C4 antibody.

In other embodiments, a scFv comprises a V_{H} domain, a V_{L} domain, and a peptide linker, wherein the V_{H} and V_{L} domains are based on the V_{H} and V_{L} domains of C23.21 antibody.

In still other embodiments, a scFv comprises a V_{H} domain, a V_{L} domain, and a peptide linker, wherein the V_{H} and V_{L} domains are based on the V_{H} and V_{L} domains of Anti-Strep-tag II antibody; StrepMAB-Immo; StrepMAB-Classic; or Strep-tag Antibody, or any combination thereof.

In further embodiments, a scFv comprises a light chain variable region (V_{L}) that is at least 90% identical to the amino acid sequence shown in SEQ ID NO:3; 10; or 16; and a heavy chain variable region (V_{H}) that is at least 90% identical to the amino acid sequence shown in SEQ ID NO:2; 8; or 14. In further embodiments, a scFv comprises a V_{L} comprising or consisting of the amino acid sequence shown in SEQ ID NO:3; 10; or 16; and a V_{H} comprising or consisting of the amino acid sequence shown in SEQ ID NO:2; 8; or 14. In additional embodiments, the scFv comprises (a) a V_{L} of SEQ ID NO:3 and a V_{H} of SEQ ID NO:2; (b) a V_{L} of SEQ ID NO:10 and a V_{H} of SEQ ID NO:8; or (c) a V_{L} of SEQ ID NO:16 and a V_{H} of SEQ ID NO:14. Any scFv of the present disclosure may be engineered so that the C-terminal end of the V_{L} domain is linked by a short peptide sequence to the N-terminal end of the V_{H} domain, or vice versa (i.e., (N)V_{L}(C)-linker-(N)V_{H}(C) or (N)V_{H}(C)-linker-(N)V_{L}(C). In specific embodiments, a scFv comprises or consists of the amino acid sequence of any one of SEQ ID NO:5, 6, 11, 12, 17, or 18.

As used herein, “specifically binds” or “specific for” refers to an association or union of a binding protein (e.g., a T cell receptor or a chimeric antigen receptor) or a binding domain (or fusion protein thereof) to a target molecule (e.g., a strep-tag peptide comprising the amino acid sequence shown in SEQ ID NO: 19) with an affinity or Kₐ (i.e., an equilibrium association constant of a particular binding interaction with units of 1/M) equal to or greater than 10⁵ M⁻¹ (which equals the ratio of the on-rate [Kₒₙ] to the off rate [K_{off}] for this association reaction), while not significantly associating or uniting with any other molecules or components in a sample. Binding proteins or binding domains (or fusion proteins thereof) may be classified as “high-affinity” binding proteins or binding domains (or fusion proteins thereof) or as “low-affinity” binding proteins or binding domains (or fusion proteins thereof). “High-affinity” binding proteins or binding domains refer to those binding proteins or binding domains having a Kₐ of at least 10⁷M⁻¹, at least 10⁸ M⁻¹, at least 10⁹ M¹, at least 10¹⁰ M⁻¹, at least 10¹¹ M⁻¹, at least 10¹²M⁻¹, or at least 10¹³ M⁻¹. “Low-affinity” binding proteins or binding domains refer to those binding proteins or binding domains having a Kₐ of up to 10⁷ M⁻¹, up to 10⁶ M⁻¹, or up to 10⁵M⁻¹. Alternatively, affinity may be defined as an equilibrium dissociation constant (Kd) of a particular binding interaction with units of M (e.g., 10⁻⁵ M to 10⁻¹³ M).

In certain embodiments, a receptor or binding domain may have “enhanced affinity,” which refers to selected or engineered receptors or binding domains with stronger binding to a target antigen than a wild type (or parent) binding domain. For example, enhanced affinity may be due to a Kₐ (equilibrium association constant) for the target antigen that is higher than the wild type binding domain, due to a K_{d} (dissociation constant) for the target antigen that is less than that of the wild type binding domain, due to an off-rate (k_{off}) for the target antigen that is less than that of the wild type binding domain, or a combination thereof. In certain embodiments, fusion proteins may be codon-optimized to enhance expression in a particular host cell, such as T cells (Scholten et al., Clin. Immunol. 119:135, 2006).

A variety of assays are known for identifying binding domains of the present disclosure that specifically bind a particular target, as well as determining binding domain or fusion protein affinities, such as Western blot, ELISA, analytical ultracentrifugation, spectroscopy and surface plasmon resonance (Biacore®) analysis (see, e.g., Scatchard et al., Ann. N.Y. Acad. Sci. 51:660, 1949; Wilson, Science 295:2103, 2002; Wolff et al., Cancer Res. 53:2560, 1993; and U.S. Pat. Nos. 5,283,173, 5,468,614, or the equivalent). Assays for assessing affinity or apparent affinity or relative affinity are also known. In certain examples, apparent affinity for a fusion protein is measured by assessing binding to various concentrations of tetramers, for example, by flow cytometry using labeled tetramers. In some examples, apparent K_{D} of a fusion protein is measured using 2-fold dilutions of labeled tetramers at a range of concentrations, followed by determination of binding curves by non-linear regression, apparent K_{D} being determined as the concentration of ligand that yielded half-maximal binding.

As used herein, an “effector domain” is an intracellular portion or domain of a fusion protein or receptor that can directly or indirectly promote a biological or physiological response in a cell when receiving an appropriate signal. In certain embodiments, an effector domain is from a protein or portion thereof or protein complex that receives a signal when bound, or when the protein or portion thereof or protein complex binds directly to a target molecule and triggers a signal from the effector domain.

An effector domain may directly promote a cellular response when it contains one or more signaling domains or motifs, such as an Intracellular Tyrosine-based Activation Motif (ITAM), as found in costimulatory molecules. Without wishing to be bound by theory, it is believed that ITAMs are important for T cell activation following ligand engagement by a T cell receptor or by a fusion protein comprising a T cell effector domain. In certain embodiments, the intracellular component or functional portion thereof comprises an ITAM. Exemplary effector domains include those from CD27, CD28, 4-1BB (CD137), OX40 (CD134), CD3ε, CD3δ, CD3ζ, CD25, CD27, CD28, CD79A, CD79B, CARD11, DAP10, FcRα, FcRβ, FcRγ, Fyn, HVEM, ICOS, Lck, LAG3, LAT, LRP, NKG2D, NOTCH1, NOTCH2, NOTCH3, NOTCH4, Wnt, ROR2, Ryk, SLAMF1, Slp76, pTα, TCRα, TCRβ, TRIM, Zap70, PTCH2, or any combination thereof. In certain embodiments, an effector domain comprises a lymphocyte receptor signaling domain (e.g., CD3ζ or a functional portion thereof).

In further embodiments, the intracellular component of the fusion protein comprises a costimulatory domain or a functional portion thereof selected from CD27, CD28, 4-1BB (CD137), OX40 (CD134), or a combination thereof. In certain embodiments, the intracellular component comprises a CD28 costimulatory domain or a functional portion thereof (which may optionally include a LL→GG mutation at positions 186-187 of the native CD28 protein (see Nguyen et al., Blood 102:4320, 2003)), a 4-1BB costimulatory domain or a functional portion thereof, or both.

In certain embodiments, an effector domain comprises CD3ζ or a functional portion thereof. In further embodiments, an effector domain comprises a portion or a domain from CD27. In further embodiments, an effector domain comprises a portion or a domain from CD28. In still further embodiments, an effector domain comprises a portion or a domain from 4-1BB. In further embodiments, an effector domain comprises a portion or a domain from OX40.

An extracellular component and an intracellular component of the present disclosure are connected by a transmembrane domain. A “transmembrane domain,” as used herein, is a portion of a transmembrane protein that can insert into or span a cell membrane. Transmembrane domains have a three-dimensional structure that is thermodynamically stable in a cell membrane and generally range in length from about 15 amino acids to about 30 amino acids. The structure of a transmembrane domain may comprise an alpha helix, a beta barrel, a beta sheet, a beta helix, or any combination thereof. In certain embodiments, the transmembrane domain comprises or is derived from a known transmembrane protein (e.g., a CD4 transmembrane domain, a CD8 transmembrane domain, a CD27 transmembrane domain, a CD28 transmembrane domain, or any combination thereof).

In certain embodiments, the extracellular component of the fusion protein further comprises a linker disposed between the binding domain and the transmembrane domain. As used herein when referring to a component of a fusion protein that connects the binding and transmembrane domains, a “linker” may be an amino acid sequence having from about two amino acids to about 500 amino acids, which can provide flexibility and room for conformational movement between two regions, domains, motifs, fragments, or modules connected by the linker. For example, a linker of the present disclosure can position the binding domain away from the surface of a host cell expressing the fusion protein to enable proper contact between the host cell and a target cell, antigen binding, and activation (Patel et al., Gene Therapy 6: 412-419, 1999). Linker length may be varied to maximize antigen recognition based on the selected target molecule, selected binding epitope, or antigen binding domain size and affinity (see, e.g., Guest et al., J. Immunother. 28:203-11, 2005; PCT Publication No. WO 2014/031687). Exemplary linkers include those having a glycine-serine amino acid chain having from one to about ten repeats of GlyₓSer_{y}, wherein x and y are each independently an integer from 0 to 10, provided that x and y are not both 0 (e.g., (Gly₄Ser)₂ (SEQ ID NO: 20); (Gly₃Ser)₂ (SEQ ID NO: 21); Gly₂Ser; or a combination thereof, such as (Gly₃Ser)₂Gly₂Ser (SEQ ID NO: 49)).

Linkers of the present disclosure also include immunoglobulin constant regions (i.e., CH1, CH2, CH3, or CL, of any isotype) and portions thereof. In certain embodiments, the linker comprises a CH3 domain, a CH2 domain, or both. In certain embodiments, the linker comprises a CH2 domain and a CH3 domain. In further embodiments, the CH2 domain and the CH3 domain are each a same isotype. In particular embodiments, the CH2 domain and the CH3 domain are an IgG4 or IgG1 isotype. In other embodiments, the CH2 domain and the CH3 domain are each a different isotype. In specific embodiments, the CH2 comprises a N297Q mutation. Without wishing to be bound by theory, it is believed that CH2 domains with N297Q mutation do not bind FcγR (see, e.g., Sazinsky et al., PNAS 105(51):20167 (2008)). In certain embodiments, the linker comprises a human immunoglobulin constant region or a portion thereof.

In any of the embodiments described herein, a linker may comprise a hinge region or a portion thereof. Hinge regions are flexible amino acid polymers of variable length and sequence (typically rich in proline and cysteine amino acids) and connect larger and less-flexible regions of immunoglobulin proteins. For example, hinge regions connect the Fc and Fab regions of antibodies and connect the constant and transmembrane regions of TCRs. In certain embodiments, the linker comprises an immunoglobulin constant region or a portion thereof and a hinge region or a portion thereof. In certain embodiments, the linker comprises a glycine-serine linker comprising or consisting of the amino acid sequence shown in SEQ ID NO: 20, or 21, or 49.

In certain embodiments, one or more of the extracellular component, the binding domain, the linker, the transmembrane domain, the intracellular component, or the costimulatory domain comprises junction amino acids. “Junction amino acids” or “junction amino acid residues” refer to one or more (e.g., about 2-20) amino acid residues between two adjacent domains, motifs, regions, modules, or fragments of a protein, such as between a binding domain and an adjacent linker, between a transmembrane domain and an adjacent extracellular or intracellular domain, or on one or both ends of a linker that links two domains, motifs, regions, modules, or fragments (e.g., between a linker and an adjacent binding domain or between a linker and an adjacent hinge). Junction amino acids may result from the construct design of a fusion protein (e.g., amino acid residues resulting from the use of a restriction enzyme site or self-cleaving peptide sequences during the construction of a polynucleotide encoding a fusion protein). For example, a transmembrane domain of a fusion protein may have one or more junction amino acids at the amino-terminal end, carboxy-terminal end, or both.

Protein tags are unique peptide sequences that are affixed or genetically fused to, or are a part of, a protein of interest and can be recognized or bound by, for example, a heterologous or non-endogenous cognate binding molecule or a substrate (e.g., receptor, ligand, antibody, carbohydrate, or metal matrix) or a fusion protein of this disclosure. Protein tags can be useful for detecting, identifying, isolating, tracking, purifying, enriching for, targeting, or biologically or chemically modifying tagged proteins of interest, particularly when a tagged protein is part of a heterogeneous population of cell proteins or cells (e.g., a biological sample like peripheral blood). In tagged cell surface proteins, the ability of the tag(s) to be specifically bound by a cognate binding molecule or a fusion protein of this disclosure (i.e., binding to a tag peptide having the amino acid sequence of SEQ ID NO: 19) is distinct from, or is in addition to, the ability of binding domain(s) contained by the cell surface protein (e.g., CAR, TCR) to specifically bind target molecule(s). In certain embodiments, a protein tag of a fusion protein of this disclosure comprises a Myc tag, His tag, Flag tag, Xpress tag, Avi tag, Calmodulin tag, Polyglutamate tag, HA tag, Nus tag, S tag, X tag, SBP tag, Softag, V5 tag, CBP, GST, MBP, GFP, Thioredoxin tag, or any combination thereof. In some embodiments, a fusion protein of the present disclosure may further comprise a protein tag (also referred to as a “peptide tag” or “tag peptide” herein), provided that the protein tag is not a strep-tag (e.g., does not comprise the amino acid sequence shown in SEQ ID NO: 19).

In any of the embodiments described herein, a fusion protein can be or can comprise a CAR or a TCR. Methods for making fusion proteins, including CARs, are described, for example, in U.S. Pat. Nos. 6,410,319; 7,446,191; U.S. Patent Publication No. 2010/065818; U.S. Pat. No. 8,822,647; PCT Publication No. WO 2014/031687; U.S. Pat. No. 7,514,537; Brentj ens et al., 2007, Clin. Cancer Res. 13:5426, and Walseng et al., Scientific Reports 7:10713, 2017, the techniques of which are herein incorporated by reference. Methods for producing engineered TCRs are described in, for example, Bowerman et al., Mol. Immunol., 46(15):3000 (2009), the techniques of which are herein incorporated by reference.

In certain embodiments, the antigen-binding fragment of the TCR comprises a single chain TCR (scTCR), which comprises both the TCR Va and VP domains TCR, but only a single TCR constant domain (Cα or Cβ). In certain embodiments, the antigen-binding fragment of the TCR, or chimeric antigen receptor is chimeric (e.g., comprises amino acid residues or motifs from more than one donor or species), humanized (e.g., comprises residues from a non-human organism that are altered or substituted so as to reduce the risk of immunogenicity in a human), or human.

Methods useful for isolating and purifying recombinantly produced soluble fusion proteins, by way of example, may include obtaining supernatants from suitable host cell/vector systems that secrete the recombinant soluble fusion protein into culture media and then concentrating the media using a commercially available filter. Following concentration, the concentrate may be applied to a single suitable purification matrix or to a series of suitable matrices, such as an affinity matrix or an ion exchange resin. One or more reverse phase HPLC steps may be employed to further purify a recombinant polypeptide. These purification methods may also be employed when isolating an immunogen from its natural environment. Methods for large scale production of one or more of the isolated/recombinant soluble fusion protein described herein include batch cell culture, which is monitored and controlled to maintain appropriate culture conditions. Purification of the soluble fusion protein may be performed according to methods described herein and known in the art and that comport with laws and guidelines of domestic and foreign regulatory agencies.

Fusion proteins as described herein may be functionally characterized according to any of a large number of art-accepted methodologies for assaying host cell (e.g., T cell) activity, including determination of T cell binding, activation or induction and also including determination of T cell responses that are antigen-specific. Examples include determination of T cell proliferation, T cell cytokine release, antigen-specific T cell stimulation, MEW restricted T cell stimulation, CTL activity (e.g., by detecting ⁵¹Cr or Europium release from pre-loaded target cells), changes in T cell phenotypic marker expression, and other measures of T-cell functions. Procedures for performing these and similar assays are may be found, for example, in Lefkovits (Immunology Methods Manual: The Comprehensive Sourcebook of Techniques, 1998). See, also, Current Protocols in Immunology; Weir, Handbook of Experimental Immunology, Blackwell Scientific, Boston, Mass. (1986); Mishell and Shigii (eds.) Selected Methods in Cellular Immunology, Freeman Publishing, San Francisco, Calif. (1979); Green and Reed, Science 281:1309 (1998) and references cited therein.

Levels of cytokines may be determined according to methods described herein and practiced in the art, including for example, ELISA, ELISPOT, intracellular cytokine staining, and flow cytometry and combinations thereof (e.g., intracellular cytokine staining and flow cytometry). Immune cell proliferation and clonal expansion resulting from an antigen-specific elicitation or stimulation of an immune response may be determined by isolating lymphocytes, such as circulating lymphocytes in samples of peripheral blood cells or cells from lymph nodes, stimulating the cells with antigen, and measuring cytokine production, cell proliferation and/or cell viability, such as by incorporation of tritiated thymidine or non-radioactive assays, such as MTT assays and the like. The effect of an immunogen described herein on the balance between a Thl immune response and a Th2 immune response may be examined, for example, by determining levels of Thl cytokines, such as IFN-γ, IL-12, IL-2, and TNF-β, and Type 2 cytokines, such as IL-4, IL-5, IL-9, IL-10, and IL-13.

### Polynucleotides, Vectors, and Host Cells

In certain aspects, nucleic acid molecules are provided that encode any one or more of the fusion proteins as described herein, which polynucleotides may be referred herein to as “anti-tag-encoding polynucleotides” and the encoded fusion proteins may be referred to herein as “anti-tag-fusion proteins.” A polynucleotide encoding a desired fusion protein of this disclosure can be inserted into an appropriate vector (e.g., viral vector or non-viral plasmid vector) for introduction into a host cell of interest (e.g., an immune cell, such as a T cell).

In certain embodiments, markers can be used to identify, monitor or isolate a host cell transduced with a heterologous polynucleotide encoding a fusion protein as provided herein. In certain embodiments, an anti-tag-encoding polynucleotide further comprises a polynucleotide that encodes a marker. In further embodiments, the polynucleotide encoding the marker is located 3′ of the polynucleotide encoding the fusion protein, or is located 5′ of the polynucleotide encoding the fusion protein. Exemplary markers include green fluorescent protein, an extracellular domain of human CD2, a truncated human EGFR (huEGFRt, (see Wang et al., Blood 118:1255, 2011), a truncated human CD19 (huCD19t); a truncated human CD34 (huCD34t); or a truncated human NGFR (huNGFRt). In certain embodiments, an encoded marker comprises EGFRt, CD19t, CD34t, or NGFRt. In any of the aforementioned embodiments, a marker may contain peptide tag, though it will be appreciated that an anti-tag fusion protein generally does not comprise a peptide tag having the same amino acid sequence as the tag to which the fusion protein binds. For example, it is preferred that an anti-tag fusion protein (or a host cell expressing the same) that binds to a tag comprising the amino acid sequence shown in SEQ ID NO:19 does not itself comprise (or, in the case of the host cell, express) a peptide having the amino acid sequence shown in SEQ ID NO:19.

In any of the embodiments described herein, an anti-tag fusion protein-encoding polynucleotide can further comprise a polynucleotide that encodes a marker and a polynucleotide that encodes a self-cleaving polypeptide, wherein the polynucleotide encoding the self-cleaving polypeptide is located between the polynucleotide encoding the fusion protein and the polynucleotide encoding the marker. When the anti-tag encoding polynucleotide, marker encoding polynucleotide, and self-cleaving polypeptide are expressed by a host cell, the fusion protein and the marker will be present on the host cell surface as separate molecules. In certain embodiments, a self-cleaving polypeptide comprises a 2A peptide from porcine teschovirus-1 (P2A; SEQ ID NO:40 or 41), Thoseaasigna virus (T2A; SEQ ID NO:42 or 43), equine rhinitis A virus (E2A; SEQ ID NO:44 or 45), or foot-and-mouth disease virus (F2A)). Further exemplary nucleic acid and amino acid sequences of 2A peptides are set forth in, for example, Kim et al. (PLOS One 6:e18556, 2011, which 2A nucleic acid and amino acid sequences are incorporated herein by reference in their entirety).

In certain embodiments, an anti-tag-encoding polynucleotide of the present disclosure comprises a V_{H}-encoding polynucleotide comprising or consisting of the nucleotide sequence set forth in any one of SEQ ID NOs:1; 7; or 13; and further comprises a V_{L}-encoding polynucleotide comprising or consisting of the nucleotide sequence set forth in any one of SEQ ID NOs:4; 9; or 15.

Representative tagged chimeric effector molecules, such as CARs containing one or more tag peptides, are described in PCT Publication No. WO 2015/095895, the tags and tagged effector molecules of which are herein incorporated by reference.

In another aspect, the present disclosure provides an anti-tag fusion protein or a cell expressing an anti-tag fusion protein on its cell surface for use in detecting or monitoring a host cell expressing a tagged cell surface protein, such as a tagged chimeric antigen receptor (CAR), a tagged T cell receptor (TCR), or a tagged marker.

For example, a host cell to be detected or monitored may express a heterologous non-tagged CAR or non-tagged TCR and further expresses a tagged marker. In certain embodiments, a polynucleotide encoding a tagged marker comprises a polynucleotide encoding the marker containing a strep-tag peptide, which strep-tag peptide may comprise or consist of the amino acid sequence shown in SEQ ID NO: 19. In certain embodiments, an immune cell to be detected or monitored may contain a chimeric polynucleotide, wherein the chimeric polynucleotide comprises a first polynucleotide encoding a heterologous cell surface receptor (such as a CAR or TCR), a second polynucleotide encoding a tagged marker comprising a polynucleotide encoding the marker containing a tag peptide, wherein the encoded tag peptide comprises a strep-tag peptide (e.g., a peptide comprising or consisting of the amino acid sequence shown in SEQ ID NO: 19), and a third polynucleotide encoding a self-cleaving polypeptide disposed between the first polynucleotide encoding the cell surface receptor and the second polynucleotide encoding the tagged marker.

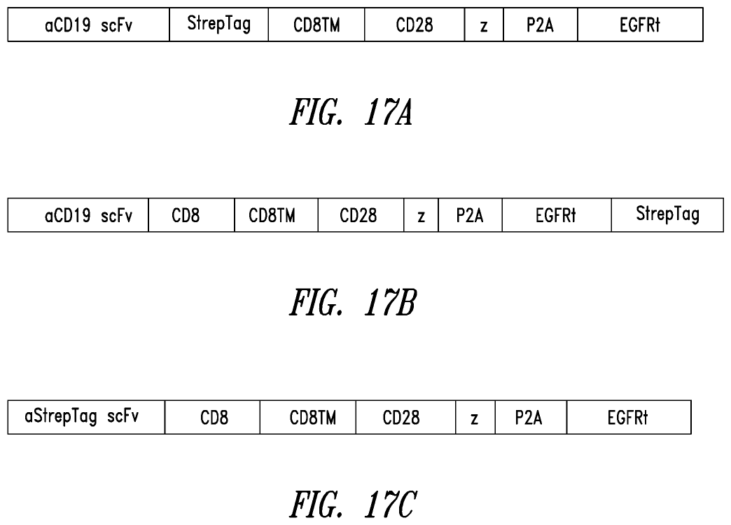

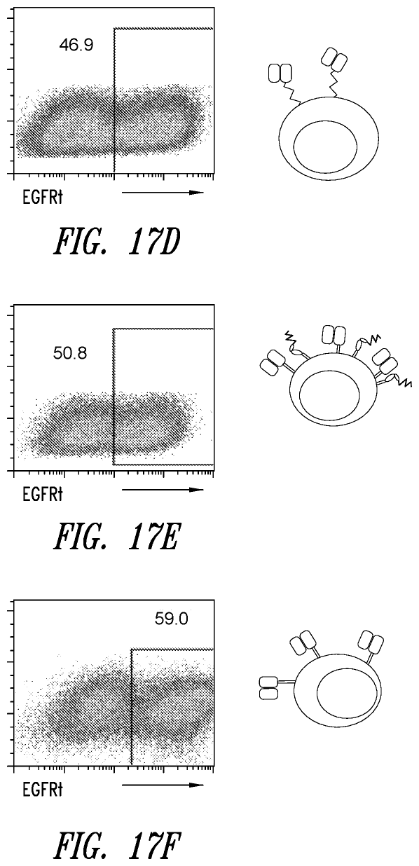

A schematic diagram of an exemplary anti-tag fusion protein-encoding polynucleotide is provided in FIG. 17C.

A schematic diagram of an exemplary polynucleotide encoding a tagged (strep-tag) cell surface receptor (CAR) specific for a target antigen (CD19) is provided in FIG. 17A. A schematic diagram of an exemplary polynucleotide encoding cell surface receptor (CAR) specific for a target antigen (CD19) and a polynucleotide encoding a tagged (strep-tag) marker (tEGFR) is provided in FIG. 17B.

In certain embodiments, a chimeric polynucleotide comprises a first polynucleotide encoding a cell surface receptor that includes (a) a first extracellular component comprising a binding domain that specifically binds to a target antigen, (b) an intracellular component comprising an effector domain or a functional portion thereof, and (c) a transmembrane component connecting the extracellular component and the intracellular component, and a second polynucleotide encodes a tagged marker comprises a polynucleotide encoding the marker containing a tag peptide, wherein the encoded tag peptide comprises a strep-tag peptide, which can, in certain embodiments, comprise or consist of the amino acid sequence shown in SEQ ID NO: 19. In further embodiments, a cell surface receptor encoded by a chimeric polynucleotide is or comprises a CAR or a TCR that specifically binds to a target antigen (e.g., a cancer antigen such as, for example, a CD19, CD20, CD22, ROR1, EGFR, EGFRvIII, EGP-2, EGP-40, GD2, GD3, HPV E6, HPV E7, Her2, L1-CAM, Lewis A, Lewis Y, MUC1, MUC16, PSCA, PSMA, CD56, CD23, CD24, CD30, CD33, CD37, CD44v7/8, CD38, CD56, CD123, CA125, c-MET, FcRH5, WT1, folate receptor α, VEGF-α, VEGFR1, VEGFR2, IL-13Rα2, IL-11Rα, MAGE-A1, MAGE-A3, MAGE-A4, SSX-2, PRAME, HA-1, PSA, ephrin A2, ephrin B2, an NKG2D, NY-ESO-1, TAG-72, mesothelin, NY-ESO, 5T4, BCMA, FAP, Carbonic anhydrase 9, ERBB2, BRAF^{V600E}, or CEA antigen).

In any of the embodiments described herein, a self-cleaving polypeptide encoded by a chimeric polynucleotide of this disclosure encodes a P2A, a T2A, an E2A, or a F2A.

In certain embodiments, an encoded tagged marker comprises EGFRt, CD19t, CD34t, or NGFRt. An encoded tagged marker may contain the tag in any position within the marker provided that the tag peptide portion of the construct can be specifically bound by a fusion protein of the present disclosure when the tagged marker is expressed at the surface of the host cell. In specific embodiments, a polynucleotide encoding the tag is located 3′ to the polynucleotide encoding the marker, or a polynucleotide encoding the tag is located 5′ to the polynucleotide encoding the marker. In other embodiments, a polynucleotide encoding the tag is located within the polynucleotide encoding the marker.

In particular embodiments, a chimeric polynucleotide comprises a structure from 5′-end to 3′ end of: (a) (the first polynucleotide encoding the cell surface receptor)-(the third polynucleotide encoding a self-cleaving polypeptide)-(the second polynucleotide encoding the tagged marker); or (b) (the second polynucleotide encoding the tagged marker)-(the third polynucleotide encoding a self-cleaving polypeptide)-(the first polynucleotide encoding the cell surface receptor).

In any of the embodiments described herein, a polynucleotide of the present disclosure (i.e., an anti-tag-fusion protein encoding polynucleotide or polynucleotide encoding a cell surface protein and a tagged marker) may be codon-optimized for a host cell containing the polynucleotide (see, e.g, Scholten et al., Clin. Immunol. 119:135-145 (2006).

In further aspects, expression constructs are provided, wherein the expression constructs comprise a polynucleotide of the present disclosure (e.g., an anti-tag-fusion protein-encoding polynucleotide or a polynucleotide encoding a cell surface protein and a tagged marker) operably linked to an expression control sequence (e.g., a promoter). In certain embodiments, the expression construct is comprised in a vector. An exemplary vector may comprise a polynucleotide capable of transporting another polynucleotide to which it has been linked, or which is capable of replication in a host organism. Some examples of vectors include plasmids, viral vectors, cosmids, and others. Some vectors may be capable of autonomous replication in a host cell into which they are introduced (e.g. bacterial vectors having a bacterial origin of replication and episomal mammalian vectors), whereas other vectors may be integrated into the genome of a host cell or promote integration of the polynucleotide insert upon introduction into the host cell and thereby replicate along with the host genome (e.g., lentiviral vector, retroviral vector). Additionally, some vectors are capable of directing the expression of genes to which they are operatively linked (these vectors may be referred to as “expression vectors”). According to related embodiments, it is further understood that, if one or more agents (e.g., polynucleotides encoding fusion proteins as described herein) are co-administered to a subject, that each agent may reside in separate or the same vectors, and multiple vectors (each containing a different agent or the same agent) may be introduced to a cell or cell population or administered to a subject.

In certain embodiments, polynucleotides of the present disclosure may be operatively linked to certain elements of a vector. For example, polynucleotide sequences that are needed to effect the expression and processing of coding sequences to which they are ligated may be operatively linked. Expression control sequences may include appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequences); sequences that enhance protein stability; and possibly sequences that enhance protein secretion. Expression control sequences may be operatively linked if they are contiguous with the gene of interest and expression control sequences that act in trans or at a distance to control the gene of interest. In certain embodiments, the vector comprises a plasmid vector or a viral vector (e.g., a vector selected from lentiviral vector or a y-retroviral vector). Viral vectors include retrovirus, adenovirus, parvovirus (e.g., adeno-associated viruses), coronavirus, negative strand RNA viruses such as ortho-myxovirus (e.g., influenza virus), rhabdovirus (e.g., rabies and vesicular stomatitis virus), paramyxovirus (e.g., measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox and canarypox). Other viruses include Norwalk virus, togavirus, flavivirus, reoviruses, papovavirus, hepadnavirus, and hepatitis virus, for example. Examples of retroviruses include avian leukosis-sarcoma, mammalian C-type, B-type viruses, D type viruses, HTLV-BLV group, lentivirus, spumavirus (Coffin, J. M., Retroviridae: The viruses and their replication, In Fundamental Virology, Third Edition, B. N. Fields et al., Eds., Lippincott-Raven Publishers, Philadelphia, 1996).

“Retroviruses” are viruses having an RNA genome, which is reverse-transcribed into DNA using a reverse transcriptase enzyme, the reverse-transcribed DNA is then incorporated into the host cell genome. “Gammaretrovirus” refers to a genus of the retroviridae family. Examples of gammaretroviruses include mouse stem cell virus, murine leukemia virus, feline leukemia virus, feline sarcoma virus, and avian reticuloendotheliosis viruses.

“Lentiviral vector,” as used herein, means HIV-based lentiviral vectors for gene delivery, which can be integrative or non-integrative, have relatively large packaging capacity, and can transduce a range of different cell types. Lentiviral vectors are usually generated following transient transfection of three (packaging, envelope and transfer) or more plasmids into producer cells. Like HIV, lentiviral vectors enter the target cell through the interaction of viral surface glycoproteins with receptors on the cell surface. On entry, the viral RNA undergoes reverse transcription, which is mediated by the viral reverse transcriptase complex. The product of reverse transcription is a double-stranded linear viral DNA, which is the substrate for viral integration into the DNA of infected cells.

In certain embodiments, the viral vector can be a gammaretrovirus, e.g., Moloney murine leukemia virus (MLV)-derived vectors. In other embodiments, the viral vector can be a more complex retrovirus-derived vector, e.g., a lentivirus-derived vector. HIV-1-derived vectors belong to this category. Other examples include lentivirus vectors derived from HIV-2, FIV, equine infectious anemia virus, SIV, and Maedi-Visna virus (ovine lentivirus). Methods of using retroviral and lentiviral viral vectors and packaging cells for transducing mammalian host cells with viral particles containing CAR transgenes are known in the art and have been previous described, for example, in: U.S. Pat. No. 8,119,772; Walchli et al., PLoS One 6:327930, 2011; Zhao et al., J. Immunol. /74:4415, 2005; Engels et al., Hum. Gene Ther. 14:1155, 2003; Frecha et al., Mol. Ther. 18:1748, 2010; and Verhoeyen et al., Methods Mol. Biol. 506:97, 2009. Retroviral and lentiviral vector constructs and expression systems are also commercially available. Other viral vectors also can be used for polynucleotide delivery including DNA viral vectors, including, for example adenovirus-based vectors and adeno-associated virus (AAV)-based vectors; vectors derived from herpes simplex viruses (HSVs), including amplicon vectors, replication-defective HSV and attenuated HSV (Krisky et al., Gene Ther. 5:1517, 1998).

Other vectors recently developed for gene therapy uses can also be used with the compositions and methods of this disclosure. Such vectors include those derived from baculoviruses and α-viruses. (Jolly, D J. 1999. Emerging Viral Vectors. pp 209-40 in Friedmann T. ed. The Development of Human Gene Therapy. New York: Cold Spring Harbor Lab), or plasmid vectors (such as sleeping beauty or other transposon vectors).

When a viral vector genome comprises a plurality of polynucleotides to be expressed in a host cell as separate transcripts, the viral vector may also comprise additional sequences between the two (or more) transcripts allowing for bicistronic or multicistronic expression. Examples of such sequences used in viral vectors include internal ribosome entry sites (IRES), furin cleavage sites, viral 2A peptide, or any combination thereof.

Construction of an expression vector that is used for genetically engineering and producing a fusion protein of interest can be accomplished by using any suitable molecular biology engineering techniques known in the art. To obtain efficient transcription and translation, a polynucleotide in each recombinant expression construct includes at least one appropriate expression control sequence (also called a regulatory sequence), such as a leader sequence and particularly a promoter operably (i.e., operatively) linked to the nucleotide sequence encoding the immunogen.

In certain embodiments, polynucleotides of the present disclosure are used to transfect/transduce a host cell (e.g., a T cell) for use in adoptive transfer therapy (e.g., targeting a cancer antigen or targeting an adoptively transferred cell that expresses a tag peptide). Methods for transfecting/transducing T cells with desired nucleic acids have been described (e.g., U.S. Patent Application Pub. No. US 2004/0087025) as have adoptive transfer procedures using T cells of desired target-specificity (e.g., Schmitt et al., Hum. Gen. 20:1240, 2009; Dossett et al., Mol. Ther. 17:742, 2009; Till et al., Blood 112:2261, 2008; Wang et al., Hum. Gene Ther. 18:712, 2007; Kuball et al., Blood 109:2331, 2007; US 2011/0243972; US 2011/0189141; Leen et al., Ann. Rev. Immunol. 25:243, 2007), such that adaptation of these methodologies to the presently disclosed embodiments is contemplated, based on the teachings herein, including those directed to fusion proteins of the present disclosure. Accordingly, in another aspect, host cells are provided that comprise a polynucleotide of the present disclosure and express the encoded fusion protein or express the encoded cell surface receptor and tagged marker. In certain embodiments, a host cell comprises: (a) a fusion protein encoding polynucleotide or fusion protein encoding expression construct of the present disclosure, wherein the host cell expresses the encoded fusion protein; or (b) a chimeric polynucleotide or chimeric polynucleotide expression construct of the present disclosure, wherein the host cell expresses the encoded cell surface receptor and the encoded tagged marker.

In certain embodiments, the host cell is a hematopoietic progenitor cell or a human immune system cell. A “hematopoietic progenitor cell”, as referred to herein, is a cell that can be derived from hematopoietic stem cells or fetal tissue and is capable of further differentiation into mature cells types (e.g., immune system cells). Exemplary hematopoietic progenitor cells include those with a CD24^{L0}Lin⁻CD117⁺ phenotype or those found in the thymus (referred to as progenitor thymocytes).

As used herein, an “immune system cell” means any cell of the immune system that originates from a hematopoietic stem cell in the bone marrow, which gives rise to two major lineages, a myeloid progenitor cell (which give rise to myeloid cells such as monocytes, macrophages, dendritic cells, megakaryocytes and granulocytes) and a lymphoid progenitor cell (which give rise to lymphoid cells such as T cells, B cells, natural killer (NK) cells, and NK-T cells). Exemplary immune system cells include a CD4⁺T cell, a CD8⁺T cell, a CD4⁻CD8⁻double negative T cell, a γδ T cell, a regulatory T cell, a stem cell memory T cell, a natural killer cell (e.g., a NK cell or a NK-T cell), a B cell, and a dendritic cell. Macrophages and dendritic cells may be referred to as “antigen presenting cells” or “APCs,” which are specialized cells that can activate T cells when a major histocompatibility complex (MHC) receptor on the surface of the APC complexed with a peptide interacts with a TCR on the surface of a T cell.

A “T cell” or “T lymphocyte” is an immune system cell that matures in the thymus and produces T cell receptors (TCRs). T cells can be naïve (not exposed to antigen; increased expression of CD62L, CCR7, CD28, CD3, CD127, and CD45RA, and decreased expression of CD45RO as compared to T_{CM}), memory T cells (T_{M}) (antigen-experienced and long-lived), and effector cells (antigen-experienced, cytotoxic). T_{M} can be further divided into subsets of central memory T cells (T_{CM}, increased expression of CD62L, CCR7, CD28, CD127, CD45RO, and CD95, and decreased expression of CD54RA as compared to naïve T cells) and effector memory T cells (T_{EM}, decreased expression of CD62L, CCR7, CD28, CD45RA, and increased expression of CD127 as compared to naïve T cells or T_{CM}).

Effector T cells (T_{E}) refers to antigen-experienced CD8⁺ cytotoxic T lymphocytes that have decreased expression of CD62L ,CCR7, CD28, and are positive for granzyme and perforin as compared to T_{CM}. Helper T cells (T_{H}) are CD4⁺ cells that influence the activity of other immune cells by releasing cytokines. CD4⁺ T cells can activate and suppress an adaptive immune response, and which of those two functions is induced will depend on presence of other cells and signals. T cells can be collected using known techniques, and the various subpopulations or combinations thereof can be enriched or depleted by known techniques, such as by affinity binding to antibodies, flow cytometry, or immunomagnetic selection. Other exemplary T cells include regulatory T cells, such as CD4⁺ CD25⁺ (Foxp3⁺) regulatory T cells and Treg17 cells, as well as Trl, Th3, CD8⁺CD28⁻, and Qa-1 restricted T cells.

“Cells of T cell lineage” refer to cells that show at least one phenotypic characteristic of a T cell, or a precursor or progenitor thereof that distinguishes the cells from other lymphoid cells, and cells of the erythroid or myeloid lineages. Such phenotypic characteristics can include expression of one or more proteins specific for T cells (e.g., CD3⁺, CD4⁺, CD8⁺), or a physiological, morphological, functional, or immunological feature specific for a T cell. For example, cells of the T cell lineage may be progenitor or precursor cells committed to the T cell lineage; CD25⁺ immature and inactivated T cells; cells that have undergone CD4 or CD8 linage commitment; thymocyte progenitor cells that are CD4⁺CD8⁺ double positive; single positive CD4⁺ or CD8⁺; TCRαP or TCR γδ; or mature and functional or activated T cells.

In certain embodiments, the immune system cell is a CD4+ T cell, a CD8+ T cell, a CD4-CD8-double negative T cell, a γδ T cell, a natural killer cell (e.g., NK cell or NK-T cell), a dendritic cell, a B cell, or any combination thereof. In certain embodiments, the immune system cell is a CD4+ T cell. In certain embodiments, the T cell is a naïve T cell, a central memory T cell, an effector memory T cell, a stem cell memory T cell, or any combination thereof.

A host cell may include any individual cell or cell culture which may receive a vector or the incorporation of nucleic acids or express proteins. The term also encompasses progeny of the host cell, whether genetically or phenotypically the same or different. Suitable host cells may depend on the vector and may include mammalian cells, animal cells, human cells, simian cells, insect cells, yeast cells, and bacterial cells. These cells may be induced to incorporate the vector or other material by use of a viral vector, transformation via calcium phosphate precipitation, DEAE-dextran, electroporation, microinjection, or other methods. See, for example, Sambrook et al., Molecular Cloning: A Laboratory Manual 2d ed. (Cold Spring Harbor Laboratory, 1989).

In any of the foregoing embodiments, a host cell that comprises a heterologous polynucleotide encoding an anti-tag fusion protein is an immune cell which is modified to reduce or eliminate expression of one or more endogenous genes that encode a polypeptide product selected from PD-1, LAG-3, CTLA4, TIM3, TIGIT, an HLA molecule, a TCR molecule, or any component or combination thereof.

Without wishing to be bound by theory, certain endogenously expressed immune cell proteins may downregulate the immune activity of a modified immune host cell (e.g., PD-1, LAG-3, CTLA4, TIGIT), or may compete with a heterologous anti-tag fusion protein of the present disclosure for expression by the host cell, or may interfere with the binding activity of a heterologously expressed binding protein of the present disclosure and interfere with the immune host cell binding to a target cell or fusion protein that expresses a tag (e.g., a tag peptide comprising the amino acid sequence shown in SEQ ID NO:19), or any combination thereof. Further, endogenous proteins (e.g., immune host cell proteins, such as an HLA) expressed on a donor immune cell to be used in a cell transfer therapy may be recognized as foreign by an allogeneic recipient, which may result in elimination or suppression of the donor immune cell by the allogeneic recipient.

Accordingly, decreasing or eliminating expression or activity of such endogenous genes or proteins can improve the activity, tolerance, and persistence of the host cells in an autologous or allogeneic host setting, and allows universal administration of the cells (e.g., to any recipient regardless of HLA type). In certain embodiments, a modified host immune cell is a donor cell (e.g., allogeneic) or an autologous cell. In certain embodiments, a modified immune host cell of this disclosure comprises a chromosomal gene knockout of one or more of a gene that encodes PD-1, LAG-3, CTLA4, TIM3, TIGIT, an HLA component (e.g., a gene that encodes an α1 macroglobulin, an α2 macroglobulin, an α3 macroglobulin, a β1 microglobulin, or a β2 microglobulin), or a TCR component (e.g., a gene that encodes a TCR variable region or a TCR constant region) (see, e.g., Torikai et al., Nature Sci. Rep. 6:21757 (2016); Torikai et al., Blood 119(24):5697 (2012); and Torikai et al., Blood 122(8):1341 (2013) the gene editing techniques, compositions, and adoptive cell therapies of which are herein incorporated by reference in their entirety). As used herein, the term “chromosomal gene knockout” refers to a genetic alteration in a host cell that prevents production, by the host cell, of a functionally active endogenous polypeptide product. Alterations resulting in a chromosomal gene knockout can include, for example, introduced nonsense mutations (including the formation of premature stop codons), missense mutations, gene deletion, and strand breaks, as well as the heterologous expression of inhibitory nucleic acid molecules that inhibit endogenous gene expression in the host cell.

In certain embodiments, a chromosomal gene knock-out or gene knock-in is made by chromosomal editing of a host cell. Chromosomal editing can be performed using, for example, endonucleases. As used herein “endonuclease” refers to an enzyme capable of catalyzing cleavage of a phosphodiester bond within a polynucleotide chain. In certain embodiments, an endonuclease is capable of cleaving a targeted gene thereby inactivating or “knocking out” the targeted gene. An endonuclease may be a naturally occurring, recombinant, genetically modified, or fusion endonuclease. The nucleic acid strand breaks caused by the endonuclease are commonly repaired through the distinct mechanisms of homologous recombination or non-homologous end joining (NHEJ). During homologous recombination, a donor nucleic acid molecule may be used for a donor gene “knock-in”, for target gene “knock-out”, and optionally to inactivate a target gene through a donor gene knock in or target gene knock out event. NHEJ is an error-prone repair process that often results in changes to the DNA sequence at the site of the cleavage, e.g., a substitution, deletion, or addition of at least one nucleotide. NHEJ may be used to “knock-out” a target gene. Examples of endonucleases include zinc finger nucleases, TALE-nucleases, CRISPR-Cas nucleases, meganucleases, and megaTALs.

As used herein, a “zinc finger nuclease” (ZFN) refers to a fusion protein comprising a zinc finger DNA-binding domain fused to a non-specific DNA cleavage domain, such as a Fokl endonuclease. Each zinc finger motif of about 30 amino acids binds to about 3 base pairs of DNA, and amino acids at certain residues can be changed to alter triplet sequence specificity (see, e.g., Desjarlais et al., Proc. Natl. Acad. Sci. 90:2256-2260, 1993; Wolfe et al., J. Mol. Biol. 285:1917-1934, 1999). Multiple zinc finger motifs can be linked in tandem to create binding specificity to desired DNA sequences, such as regions having a length ranging from about 9 to about 18 base pairs. By way of background, ZFNs mediate genome editing by catalyzing the formation of a site-specific DNA double strand break (DSB) in the genome, and targeted integration of a transgene comprising flanking sequences homologous to the genome at the site of DSB is facilitated by homology directed repair. Alternatively, a DSB generated by a ZFN can result in knock out of target gene via repair by non-homologous end joining

(NHEJ), which is an error-prone cellular repair pathway that results in the insertion or deletion of nucleotides at the cleavage site. In certain embodiments, a gene knockout comprises an insertion, a deletion, a mutation or a combination thereof, made using a ZFN molecule.

As used herein, a “transcription activator-like effector nuclease” (TALEN) refers to a fusion protein comprising a TALE DNA-binding domain and a DNA cleavage domain, such as a Fokl endonuclease. A “TALE DNA binding domain” or “TALE” is composed of one or more TALE repeat domains/units, each generally having a highly conserved 33-35 amino acid sequence with divergent 12th and 13th amino acids. The TALE repeat domains are involved in binding of the TALE to a target DNA sequence. The divergent amino acid residues, referred to as the Repeat Variable Diresidue (RVD), correlate with specific nucleotide recognition. The natural (canonical) code for DNA recognition of these TALEs has been determined such that an HD (histine-aspartic acid) sequence at positions 12 and 13 of the TALE leads to the TALE binding to cytosine (C), NG (asparagine-glycine) binds to a T nucleotide, NI (asparagine-isoleucine) to A, NN (asparagine-asparagine) binds to a G or A nucleotide, and NG (asparagine-glycine) binds to a T nucleotide. Non-canonical (atypical) RVDs are also known (see, e.g., U.S. Patent Publication No. US 2011/0301073, which atypical RVDs are incorporated by reference herein in their entirety). TALENs can be used to direct site-specific double-strand breaks (DSB) in the genome of T cells. Non-homologous end joining (NHEJ) ligates DNA from both sides of a double-strand break in which there is little or no sequence overlap for annealing, thereby introducing errors that knock out gene expression. Alternatively, homology directed repair can introduce a transgene at the site of DSB providing homologous flanking sequences are present in the transgene. In certain embodiments, a gene knockout comprises an insertion, a deletion, a mutation or a combination thereof, and made using a TALEN molecule.

As used herein, a “clustered regularly interspaced short palindromic repeats/Cas” (CRISPR/Cas) nuclease system refers to a system that employs a CRISPR RNA (crRNA)-guided Cas nuclease to recognize target sites within a genome (known as protospacers) via base-pairing complementarity and then to cleave the DNA if a short, conserved protospacer associated motif (PAM) immediately follows 3′ of the complementary target sequence. CRISPR/Cas systems are classified into three types (i.e., type I, type II, and type III) based on the sequence and structure of the Cas nucleases. The crRNA-guided surveillance complexes in types I and III need multiple Cas subunits. Type II system, the most studied, comprises at least three components: an RNA-guided Cas9 nuclease, a crRNA, and a trans-acting crRNA (tracrRNA). The tracrRNA comprises a duplex forming region. A crRNA and a tracrRNA form a duplex that is capable of interacting with a Cas9 nuclease and guiding the Cas9/crRNA:tracrRNA complex to a specific site on the target DNA via Watson-Crick base-pairing between the spacer on the crRNA and the protospacer on the target DNA upstream from a PAM. Cas9 nuclease cleaves a double-stranded break within a region defined by the crRNA spacer. Repair by NHEJ results in insertions and/or deletions which disrupt expression of the targeted locus. Alternatively, a transgene with homologous flanking sequences can be introduced at the site of DSB via homology directed repair. The crRNA and tracrRNA can be engineered into a single guide RNA (sgRNA or gRNA) (see, e.g., Jinek et al., Science 337:816-21, 2012). Further, the region of the guide RNA complementary to the target site can be altered or programed to target a desired sequence (Xie et al., PLOS One 9:e100448, 2014; U.S. Pat. Appl. Pub. No. US 2014/0068797, U.S. Pat. Appl. Pub. No. US 2014/0186843; U.S. Pat. No. 8,697,359, and PCT Publication No. WO 2015/071474; each of which is incorporated by reference). In certain embodiments, a gene knockout comprises an insertion, a deletion, a mutation or a combination thereof, and made using a CRISPR/Cas nuclease system.

Exemplary gRNA sequences and methods of using the same to knock out endogenous genes that encode immune cell proteins include those described in Ren et al., Clin. Cancer Res. 23(9):2255-2266 (2017), the gRNAs, CAS9 DNAs, vectors, and gene knockout techniques of which are hereby incorporated by reference in their entirety.

As used herein, a “meganuclease,” also referred to as a “homing endonuclease,” refers to an endodeoxyribonuclease characterized by a large recognition site (double stranded DNA sequences of about 12 to about 40 base pairs). Meganucleases can be divided into five families based on sequence and structure motifs: LAGLIDADG, GIY-YIG, HNH, His-Cys box and PD-(D/E)XK. Exemplary meganucleases include I-SceI, I-CeuI, PI-Pspl, PI-Sce, I-SceIV, I-Csml, I-PanI, I-SceII, I-Ppol, I-SceIII, I-CreI, I-TevI, I-TevII and I-TevIII, whose recognition sequences are known (see, e.g., U.S. Pat. Nos. 5,420,032 and 6,833,252; Belfort et al., Nucleic Acids Res. 25:3379-3388, 1997; Dujon et al., Gene 82:115-118, 1989; Perler et al., Nucleic Acids Res. 22:1125-1127, 1994; Jasin, Trends Genet. 12:224-228, 1996; Gimble et al., J. Mol. Biol. 263:163-180, 1996; Argast et al., J. Mol. Biol. 280:345-353, 1998).

In certain embodiments, naturally-occurring meganucleases may be used to promote site-specific genome modification of a target selected from PD-1, LAG3, TIM3, CTLA4, TIGIT, an HLA-encoding gene, or a TCR component-encoding gene. In other embodiments, an engineered meganuclease having a novel binding specificity for a target gene is used for site-specific genome modification (see, e.g., Porteus et al., Nat. Biotechnol. 23:967-73, 2005; Sussman et al., J. Mol. Biol. 342:31-41, 2004; Epinat et al., Nucleic Acids Res. 31:2952-62, 2003; Chevalier et al., Molec. Cell 10:895-905, 2002; Ashworth et al., Nature 441:656-659, 2006; Paques et al., Curr. Gene Ther. 7:49-66, 2007; U.S. Patent Publication Nos. US 2007/0117128; US 2006/0206949; US 2006/0153826; US 2006/0078552; and US 2004/0002092). In further embodiments, a chromosomal gene knockout is generated using a homing endonuclease that has been modified with modular DNA binding domains of TALENs to make a fusion protein known as a megaTAL. MegaTALs can be utilized to not only knock-out one or more target genes, but to also introduce (knock in) heterologous or exogenous polynucleotides when used in combination with an exogenous donor template encoding a polypeptide of interest.

In certain embodiments, a chromosomal gene knockout comprises an inhibitory nucleic acid molecule that is introduced into a host cell (e.g., an immune cell) comprising a heterologous polynucleotide encoding an antigen-specific receptor that specifically binds to a tumor associated antigen, wherein the inhibitory nucleic acid molecule encodes a target-specific inhibitor and wherein the encoded target-specific inhibitor inhibits endogenous gene expression (i.e., of PD-1, TIM3, LAG3, CTLA4, TIGIT, an HLA component, or a TCR component, or any combination thereof) in the host immune cell.

A chromosomal gene knockout can be confirmed directly by DNA sequencing of the host immune cell following use of the knockout procedure or agent. Chromosomal gene knockouts can also be inferred from the absence of gene expression (e.g., the absence of an mRNA or polypeptide product encoded by the gene) following the knockout.

In other aspects, kits are provided comprising (a) a vector or an expression construct as described herein and (b) reagents for transducing the vector or the expression construct into a host cell.

### Uses

The present disclosure also provides methods of modulating (e.g., ablating, stimulating, or activating) modified cells as described herein (e.g., CAR T cells that target a tag peptide, or CAR T cells that are tagged with a tag peptide). In certain embodiments, methods are provided for targeted ablation of tagged cells, wherein the methods comprise administering to a subject an immune cell modified to express on its cell surface an anti-tag fusion protein of the present disclosure, wherein the subject had been previously administered a cell expressing a cell surface protein comprising a tag peptide (which cell may be referred to herein as a “tagged cell”), the tag peptide being a strep-tag peptide (e.g., a peptide comprising or consisting of the amino acid sequence shown in SEQ ID NO: 19), thereby inducing a targeted immune response that ablates the tagged cell(s).

Such ablation methods may be useful where the previously administered tagged cells (e.g., administered for immunotherapy treatment of a disease such as a cancer, including, for example, a B cell cancer) have an undesirable activity (e.g., elicit an immune response against off-target cells or tissues in the subject) or level of activity (e.g., elicit an immune response of inappropriately high strength, duration, or both, e.g., a cytokine release syndrome (CRS) event). In certain embodiments, the modified immune cells expressing the anti-tag fusion protein are administered to the subject having at least one adverse event associated with the presence of the tagged cells.

In certain embodiments, the tagged cell surface protein comprises a CAR, a TCR, or a marker. In certain embodiments, the marker comprises EGFRt, CD19t, CD34t, or NGFRt. In certain embodiments, the tag peptide is contained in the marker.

In any of the aforementioned embodiments, the modified immune cell expressing the anti-tag fusion protein is selected from a T cell, a NK cell, or a NK-T cell. In particular embodiments, the immune cell is a T cell.

In certain embodiments, the tagged cells were previously administered to the subject as an immunotherapy, a graft, or a transplant. In particular embodiments, the tagged cells or the modified immune cells expressing the anti-tag fusion protein are allogeneic, autologous, or syngeneic to the subject. In further embodiments, the subject has or is suspected of having graft-versus-host disease (GvHD) or host-versus-graft disease (HvGD) following an immunotherapy, graft, or transplant comprising the tagged cells. In certain embodiments, the tagged cells were administered to treat a hyperproliferative disorder. As used herein, “hyperproliferative disorder” refers to excessive growth or proliferation as compared to a normal or undiseased cell. Exemplary hyperproliferative disorders include tumors, cancers, neoplastic tissue, carcinoma, sarcoma, malignant cells, pre-malignant cells, as well as non-neoplastic or non-malignant hyperproliferative disorders (e.g., adenoma, fibroma, lipoma, leiomyoma, hemangioma, fibrosis, restenosis, as well as autoimmune diseases such as rheumatoid arthritis, osteoarthritis, psoriasis, inflammatory bowel disease, or the like).

Furthermore, “cancer” may refer to any accelerated proliferation of cells, including solid tumors, ascites tumors, blood or lymph or other malignancies; connective tissue malignancies; metastatic disease; minimal residual disease following transplantation of organs or stem cells; multi-drug resistant cancers, primary or secondary malignancies, angiogenesis related to malignancy, or other forms of cancer.

Ablation of the tagged cells (i.e., of the tagged immunotherapy or tagged non-immunotherapy cells) may be determined necessary when the subject evidences one or more adverse effects associated with the tagged cells. For example, inflammation, fever, pulmonary or cerebral edema, changes in blood pressure or heart rate, undesirably low counts of healthy cells (e.g., white blood cells), undesirably high counts of tagged cells, elevated levels of cytokines, rash, blisters, jaundice, diarrhea, vomiting, abdominal cramps, fatigue, pain, stiffness, shortness of breath, weight loss, dry eyes or vision changes, dry mouth, vaginal dryness, and muscle weakness may be indicators that ablation of the tagged cells is required.

The ability of the modified immune cells expressing the anti-tag fusion protein to cause ablation of the tagged cells may be determined, either directly or indirectly, following treatment with the modified immune cells. In certain embodiments, the methods further comprise, after administering to the subject the modified immune cell, detecting the presence and/or measuring the quantity of: (i) the tagged cells remaining in the subject or in a sample obtained from the subject; (ii) the modified immune cells present in the subject or in a sample obtained from the subject; (iii) one or more cytokines in the subject; or (iv) any combination thereof. In specific embodiments, the methods further comprise detecting the presence and/or monitoring the quantity of cells that were reduced following administration of the tagged cells (e.g., healthy CD19-expressing B cells that were reduced following administration of tagged anti-CD19 CART cells).

Subjects that can be treated by the present invention are, in general, human and other primate subjects, such as monkeys and apes for veterinary medicine purposes. In any of the aforementioned embodiments, the subject may be a human subject. The subjects can be male or female and can be any suitable age, including infant, juvenile, adolescent, adult, and geriatric subjects. Cells according to the present disclosure may be administered in a manner appropriate to the disease, condition, or disorder to be treated as determined by persons skilled in the medical art. In any of the above embodiments, a cell comprising a fusion protein as described herein is administered intravenously, intraperitoneally, intratumorally, into the bone marrow, into a lymph node, or into the cerebrospinal fluid so as to encounter the tagged cells to be ablated. An appropriate dose, suitable duration, and frequency of administration of the compositions will be determined by such factors as a condition of the patient; size, type, and severity of the disease, condition, or disorder; the undesired type or level or activity of the tagged cells, the particular form of the active ingredient; and the method of administration.

In any of the above embodiments, methods of the present disclosure comprise administering a host cell expressing a fusion protein of the present disclosure. The amount of cells in a composition is at least one cell (for example, one fusion protein-modified CD8⁺ T cell subpopulation; one fusion protein-modified CD4⁺ T cell subpopulation) or is more typically greater than 10² cells, for example, up to 10⁶, up to 10⁷, up to 10⁸ cells, up to 10⁹ cells, or more than 10¹⁰ cells. In certain embodiments, the cells are administered in a range from about 10⁶ to about 10¹⁰ cells/m2, preferably in a range of about 10⁵ to about 10⁹ cells/m². The number of cells will depend upon the ultimate use for which the composition is intended as well the type of cells included therein. For example, cells modified to contain a fusion protein specific for a particular antigen will comprise a cell population containing at least 30%, 35%, 40%, 45%, 50%, 55%, 60%, 65%, 70%, 75%, 80%, 85%, 90%, 95% or more of such cells. For uses provided herein, cells are generally in a volume of a liter or less, 500 mls or less, 250 mls or less, or 100 mls or less. In embodiments, the density of the desired cells is typically greater than 10⁴ cells/ml and generally is greater than 10⁷ cells/ml, generally 10⁸ cells/ml or greater. The cells may be administered as a single infusion or in multiple infusions over a range of time. A clinically relevant number of immune cells can be apportioned into multiple infusions that cumulatively equal or exceed 10⁶, 10⁷, 10⁸, 10⁹, 10¹⁰, or 10¹¹ cells.

Unit doses are also provided herein which comprise a host cell (e.g., a modified immune cell comprising a polynucleotide of the present disclosure) or host cell composition of this disclosure. In certain embodiments, a unit dose comprises (i) a composition comprising at least about 30%, at least about 40%, at least about 50%, at least about 60%, at least about 70%, at least about 80%, at least about 85%, at least about 90%, or at least about 95% modified CD4⁺ T cells, combined with (ii) a composition comprising at least about 30%, at least about 40%, at least about 50%, at least about 60%, at least about 70%, at least about 80%, at least about 85%, at least about 90%, or at least about 95% modified CD8⁺ T cells, in about a 1:1 ratio, wherein the unit dose contains a reduced amount or substantially no naïve T cells (i.e., has less than about 50%, less than about 40%, less than about 30%, less than about 20%, less than about 10%, less than about 5%, or less then about 1% the population of naïve T cells present in a unit dose as compared to a patient sample having a comparable number of PBMCs).

In some embodiments, a unit dose comprises (i) a composition comprising at least about 50% modified CD4⁺ T cells, combined with (ii) a composition comprising at least about 50% modified CD8⁺ T cells, in about a 1:1 ratio, wherein the unit dose contains a reduced amount or substantially no naïve T cells. In further embodiments, a unit dose comprises (i) a composition comprising at least about 60% modified CD4⁺ T cells, combined with (ii) a composition comprising at least about 60% modified CD8⁺ T cells, in about a 1:1 ratio, wherein the unit dose contains a reduced amount or substantially no naïve T cells. In still further embodiments, a unit dose comprises (i) a composition comprising at least about 70% modified CD4⁺ T cells, combined with (ii) a composition comprising at least about 70% modified CD8⁺ T cells, in about a 1:1 ratio, wherein the unit dose contains a reduced amount or substantially no naïve T cells. In some embodiments, a unit dose comprises (i) a composition comprising at least about 80% modified CD4⁺ T cells, combined with (ii) a composition comprising at least about 80% modified CD8⁺ T cells, in about a 1:1 ratio, wherein the unit dose contains a reduced amount or substantially no naïve T cells. In some embodiments, a unit dose comprises (i) a composition comprising at least about 85% modified CD4⁺ T cells, combined with (ii) a composition comprising at least about 85% modified CD8⁺ T cells, in about a 1:1 ratio, wherein the unit dose contains a reduced amount or substantially no naïve T cells. In some embodiments, a unit dose comprises (i) a composition comprising at least about 90% modified CD4⁺ T cells, combined with (ii) a composition comprising at least about 90% modified CD8⁺ T cells, in about a 1:1 ratio, wherein the unit dose contains a reduced amount or substantially no naïve T cells.

In any of the embodiments described herein, a unit dose comprises equal, or approximately equal numbers of engineered CD45RA⁻ CD3⁺ CD8⁺ and engineered CD45RA⁻ CD3⁺ CD4⁺ T_{M} cells.

Also contemplated are pharmaceutical compositions that comprise fusion proteins or cells expressing the fusion proteins as disclosed herein and a pharmaceutically acceptable carrier, diluents, or excipient. Suitable excipients include water, saline, dextrose, glycerol, or the like and combinations thereof. In embodiments, compositions comprising fusion proteins or host cells as disclosed herein further comprise a suitable infusion media. Suitable infusion media can be any isotonic medium formulation, typically normal saline, Normosol R (Abbott) or Plasma-Lyte A (Baxter), 5% dextrose in water, Ringer's lactate can be utilized. An infusion medium can be supplemented with human serum albumin or other human serum components.

Pharmaceutical compositions may be administered in a manner appropriate to the disease or condition to be treated (or prevented) as determined by persons skilled in the medical art. An appropriate dose and a suitable duration and frequency of administration of the compositions will be determined by such factors as the health condition of the patient, size of the patient (i.e., weight, mass, or body area), the type and severity of the patient's condition, the undesired type or level or activity of the tagged cells, the particular form of the active ingredient, and the method of administration. In general, an appropriate dose and treatment regimen provide the composition(s) in an amount sufficient to provide therapeutic and/or prophylactic benefit (such as described herein, including an improved clinical outcome, such as more frequent complete or partial remissions, or longer disease-free and/or overall survival, or a lessening of symptom severity). For prophylactic use, a dose should be sufficient to prevent, delay the onset of, or diminish the severity of a disease associated with disease or disorder. Prophylactic benefit of the immunogenic compositions administered according to the methods described herein can be determined by performing pre-clinical (including in vitro and in vivo animal studies) and clinical studies and analyzing data obtained therefrom by appropriate statistical, biological, and clinical methods and techniques, all of which can readily be practiced by a person skilled in the art.

Certain methods of treatment or prevention contemplated herein include administering a host cell (which may be autologous, allogeneic or syngeneic) comprising a desired polynucleotide as described herein that is stably integrated into the chromosome of the cell. For example, such a cellular composition may be generated ex vivo using autologous, allogeneic or syngeneic immune system cells (e.g., T cells, antigen-presenting cells, natural killer cells) in order to administer a desired, fusion protein-expressing T-cell composition to a subject as an adoptive immunotherapy. In certain embodiments, the host cell is a hematopoietic progenitor cell or a human immune cell. In certain embodiments, the immune system cell is a CD4⁺ T cell, a CD8⁺ T cell, a CD4⁻ CD8⁻ double-negative T cell, a γϵ T cell, a natural killer cell, a dendritic cell, or any combination thereof. In certain embodiments, the immune system cell is a naïve T cell, a central memory T cell, a stem cell memory T cell, an effector memory T cell, or any combination thereof. In particular embodiments, the cell is a CD4⁺ T cell. In particular embodiments, the cell is a CD8⁺ T cell.

As used herein, administration of a composition refers to delivering the same to a subject, regardless of the route or mode of delivery. Administration may be effected continuously or intermittently, and parenterally. Administration may be for treating a subject already confirmed as having a recognized condition, disease or disease state, or for treating a subject susceptible to or at risk of developing such a condition, disease or disease state. Co-administration with an adjunctive therapy may include simultaneous and/or sequential delivery of multiple agents in any order and on any dosing schedule (e.g., fusion protein-expressing recombinant (i.e., engineered) host cells with one or more cytokines; immunosuppressive therapy such as calcineurin inhibitors, corticosteroids, microtubule inhibitors, low dose of a mycophenolic acid prodrug, or any combination thereof).

In certain embodiments, a plurality of doses of a recombinant host cell as described herein is administered to the subject, which may be administered at intervals between administrations of about two to about four weeks.

In still further embodiments, the subject being treated is further receiving immunosuppressive therapy, such as calcineurin inhibitors, corticosteroids, microtubule inhibitors, low dose of a mycophenolic acid prodrug, or any combination thereof. In yet further embodiments, the subject being treated has received a non-myeloablative or a myeloablative hematopoietic cell transplant, wherein the treatment may be administered at least two to at least three months after the non-myeloablative hematopoietic cell transplant and wherein the transplanted cells may optionally be tagged with a peptide having the amino acid sequence shown in SEQ ID NO:19.

An effective amount of a pharmaceutical composition refers to an amount sufficient, at dosages and for periods of time needed, to achieve the desired clinical results or beneficial treatment, as described herein. An effective amount may be delivered in one or more administrations. If the administration is to a subject already known or confirmed to have a disease or disease-state, the term “therapeutic amount” may be used in reference to treatment, whereas “prophylactically effective amount” may be used to describe administrating an effective amount to a subject that is susceptible or at risk of developing a disease or disease-state (e.g., recurrence) as a preventative course.

The level of a CTL immune response may be determined by any one of numerous immunological methods described herein and routinely practiced in the art. The level of a CTL immune response may be determined prior to and following administration of any one of the herein described fusion proteins expressed by, for example, a T cell. Cytotoxicity assays for determining CTL activity may be performed using any one of several techniques and methods routinely practiced in the art (see, e.g., Henkart et al., “Cytotoxic T-Lymphocytes” in Fundamental Immunology, Paul (ed.) (2003 Lippincott Williams & Wilkins, Philadelphia, Pa.), pages 1127-50, and references cited therein).

Antigen-specific T cell responses are typically determined by comparisons of observed T cell responses according to any of the herein described T cell functional parameters (e.g., proliferation, cytokine release, CTL activity, altered cell surface marker phenotype, etc.) that may be made between T cells that are exposed to a cognate antigen in an appropriate context (e.g., the antigen used to prime or activate the T cells, when presented by immunocompatible antigen-presenting cells) and T cells from the same source population that are exposed instead to a structurally distinct or irrelevant control antigen. A response to the cognate antigen that is greater, with statistical significance, than the response to the control antigen signifies antigen-specificity.

A biological sample may be obtained from a subject for determining the presence and level of an immune response to a tagged protein or cell as described herein. A “biological sample” as used herein may be a blood sample (from which serum or plasma may be prepared), biopsy specimen, body fluids (e.g., lung lavage, ascites, mucosal washings, synovial fluid), bone marrow, lymph nodes, tissue explant, organ culture, or any other tissue or cell preparation from the subject or a biological source. Biological samples may also be obtained from the subject prior to receiving any immunogenic composition, which biological sample is useful as a control for establishing baseline (i.e., pre-immunization) data.

The pharmaceutical compositions described herein may be presented in unit-dose or multi-dose containers, such as sealed ampoules or vials. Such containers may be frozen to preserve the stability of the formulation until. In certain embodiments, a unit dose comprises a recombinant host cell as described herein at a dose of about 10⁷ cells/m² to about 10¹¹ cells/m². The development of suitable dosing and treatment regimens for using the particular compositions described herein in a variety of treatment regimens, including e.g., parenteral or intravenous administration or formulation.

If the subject composition is administered parenterally, the composition may also include sterile aqueous or oleaginous solution or suspension. Suitable non-toxic parenterally acceptable diluents or solvents include water, Ringer's solution, isotonic salt solution, 1,3-butanediol, ethanol, propylene glycol or polythethylene glycols in mixtures with water. Aqueous solutions or suspensions may further comprise one or more buffering agents, such as sodium acetate, sodium citrate, sodium borate or sodium tartrate. Of course, any material used in preparing any dosage unit formulation should be pharmaceutically pure and substantially non-toxic in the amounts employed. In addition, the active compounds may be incorporated into sustained-release preparation and formulations. Dosage unit form, as used herein, refers to physically discrete units suited as unitary dosages for the subject to be treated; each unit may contain a predetermined quantity of recombinant cells or active compound calculated to produce the desired effect in association with an appropriate pharmaceutical carrier.

In general, an appropriate dosage and treatment regimen provides the active molecules or cells in an amount sufficient to provide therapeutic or prophylactic benefit. Such a response can be monitored by establishing an improved clinical outcome (e.g., more frequent remissions, complete or partial, or longer disease-free survival) in treated subjects as compared to non-treated subjects. Increases in preexisting immune responses to a tumor protein generally correlate with an improved clinical outcome. Such immune responses may generally be evaluated using standard proliferation, cytotoxicity or cytokine assays, which are routine in the art and may be performed using samples obtained from a subject before and after treatment.

In further aspects, kits are provided that comprise (a) a host cell, (b) a composition, or (c) a unit dose as described herein. In certain embodiments, a kit comprises (1) a unit dose of a tagged cell and (2) a modified immune cell expressing a fusion protein specific for a strep-tag peptide, which strep-tag peptide can, in certain embodiments, comprise or consist of the amino acid sequence shown in SEQ ID NO:19. In other words, a kit may provide both a tagged cell for use in an immunotherapy, a graft, or a transplant, as well as a modified immune cell that can target the tagged cell for modulation (e.g., ablation), if needed.

Methods according to this disclosure may further include administering one or more additional agents to treat the disease or disorder in a combination therapy. For example, in certain embodiments, a combination therapy comprises administering a fusion protein (or an engineered host cell expressing the same) with (concurrently, simultaneously, or sequentially) an immune checkpoint inhibitor. In some embodiments, a combination therapy comprises administering fusion protein of the present disclosure (or an engineered host cell expressing the same) with an agonist of a stimulatory immune checkpoint agent. In further embodiments, a combination therapy comprises administering a fusion protein of the present disclosure (or an engineered host cell expressing the same) with a secondary therapy, such as chemotherapeutic agent, a radiation therapy, a surgery, an antibody, or any combination thereof.

As used herein, the term “immune suppression agent” or “immunosuppression agent” refers to one or more cells, proteins, molecules, compounds or complexes providing inhibitory signals to assist in controlling or suppressing an immune response.

For example, immune suppression agents include those molecules that partially or totally block immune stimulation; decrease, prevent or delay immune activation; or increase, activate, or up regulate immune suppression. Exemplary immunosuppression agents to target (e.g., with an immune checkpoint inhibitor) include PD-1, PD-L1, PD-L2, LAG3, CTLA4, B7-H3, B7-H4, CD244/2B4, HVEM, BTLA, CD160, TIM3,

GALS, KIR, PVR1G (CD112R), PVRL2, adenosine, A2aR, immunosuppressive cytokines (e.g., IL-10, IL-4, IL-1RA, IL-35), IDO, arginase, VISTA, TIGIT, LAIR1, CEACAM-1, CEACAM-3, CEACAM-5, Treg cells, or any combination thereof.

An immune suppression agent inhibitor (also referred to as an immune checkpoint inhibitor) may be a compound, an antibody, an antibody fragment or fusion polypeptide (e.g., Fc fusion, such as CTLA4-Fc or LAG3-Fc), an antisense molecule, a ribozyme or RNAi molecule, or a low molecular weight organic molecule. In any of the embodiments disclosed herein, a method may comprise administering a fusion protein of the present disclosure (or an engineered host cell expressing the same) with one or more inhibitor of any one of the following immune suppression components, singly or in any combination.

In certain embodiments, a fusion protein is used in combination with a PD-1 inhibitor, for example a PD-1-specific antibody or binding fragment thereof, such as pidilizumab, nivolumab (Keytruda, formerly MDX-1106), pembrolizumab (Opdivo, formerly MK-3475), MEDI0680 (formerly AMP-514), AMP-224, BMS-936558 or any combination thereof. In further embodiments, a fusion protein of the present disclosure (or an engineered host cell expressing the same) is used in combination with a PD-L1 specific antibody or binding fragment thereof, such as BMS-936559, durvalumab (MEDI4736), atezolizumab (RG7446), avelumab (MSB0010718C), MPDL3280A, or any combination thereof.

In certain embodiments, a fusion protein of the present disclosure (or an engineered host cell expressing the same) is used in combination with a LAG3 inhibitor, such as LAG525, IMP321, IMP701, 9H12, BMS-986016, or any combination thereof.

In certain embodiments, a fusion protein is used in combination with an inhibitor of CTLA4. In particular embodiments, a fusion protein of the present disclosure (or an engineered host cell expressing the same) is used in combination with a CTLA4 specific antibody or binding fragment thereof, such as ipilimumab, tremelimumab, CTLA4-Ig fusion proteins (e.g., abatacept, belatacept), or any combination thereof.

In certain embodiments, a fusion protein of the present disclosure (or an engineered host cell expressing the same) is used in combination with a B7-H3 specific antibody or binding fragment thereof, such as enoblituzumab (MGA271), 376.96, or both. A B7-H4 antibody binding fragment may be a scFv or fusion protein thereof, as described in, for example, Dangaj et al., Cancer Res. 73:4820, 2013, as well as those described in U.S. Pat. No. 9,574,000 and PCT Patent Publication Nos. WO/201640724A1 and WO 2013/025779A1.

In certain embodiments, a fusion protein of the present disclosure (or an engineered host cell expressing the same) is used in combination with an inhibitor of CD244.

In certain embodiments, a fusion protein of the present disclosure (or an engineered host cell expressing the same) is used in combination with an inhibitor of BLTA, HVEM, CD160, or any combination thereof. Anti CD-160 antibodies are described in, for example, PCT Publication No. WO 2010/084158.

In certain embodiments, a fusion protein of the present disclosure (or an engineered host cell expressing the same) is used in combination with an inhibitor of TIM3.

In certain embodiments, a fusion protein of the present disclosure (or an engineered host cell expressing the same) is used in combination with an inhibitor of Gal9.

In certain embodiments, a fusion protein of the present disclosure (or an engineered host cell expressing the same) is used in combination with an inhibitor of adenosine signaling, such as a decoy adenosine receptor.

In certain embodiments, a fusion protein of the present disclosure (or an engineered host cell expressing the same) is used in combination with an inhibitor of A2aR. In certain embodiments, a fusion protein of the present disclosure (or an engineered host cell expressing the same) is used in combination with an inhibitor of KIR, such as lirilumab (BMS-986015).

In certain embodiments, a fusion protein of the present disclosure (or an engineered host cell expressing the same) is used in combination with an inhibitor of an inhibitory cytokine (typically, a cytokine other than TGFβ) or Treg development or activity.

In certain embodiments, a fusion protein of the present disclosure (or an engineered host cell expressing the same) is used in combination with an IDO inhibitor, such as levo-1-methyl tryptophan, epacadostat (INCB024360; Liu et al., Blood 115:3520-30, 2010), ebselen (Terentis et al., Biochem. 49:591-600, 2010), indoximod, NLG919 (Mautino et al., American Association for Cancer Research 104th Annual Meeting 2013; Apr 6-10, 2013), 1-methyl-tryptophan (1-MT)-tira-pazamine, or any combination thereof.

In certain embodiments, a fusion protein of the present disclosure (or an engineered host cell expressing the same) is used in combination with an arginase inhibitor, such as N(omega)-Nitro-L-arginine methyl ester (L-NAME), N-omega-hydroxy-nor-1-arginine (nor-NOHA), L-NOHA, 2(S)-amino-6-boronohexanoic acid (ABH), S-(2-boronoethyl)-L-cysteine (BEC), or any combination thereof.

In certain embodiments, a fusion protein of the present disclosure (or an engineered host cell expressing the same) is used in combination with an inhibitor of VISTA, such as CA-170 (Curis, Lexington, Mass.).

In certain embodiments, a fusion protein of the present disclosure (or an engineered host cell expressing the same) is used in combination with an inhibitor of TIGIT such as, for example, COM902 (Compugen, Toronto, Ontario Canada), an inhibitor of CD155, such as, for example, COM701 (Compugen), or both.

In certain embodiments, a fusion protein of the present disclosure (or an engineered host cell expressing the same) is used in combination with an inhibitor of PVRIG, PVRL2, or both. Anti-PVRIG antibodies are described in, for example, PCT Publication No. WO 2016/134333. Anti-PVRL2 antibodies are described in, for example, PCT Publication No. WO 2017/021526.

In certain embodiments, a fusion protein of the present disclosure (or an engineered host cell expressing the same) is used in combination with a LAIR1 inhibitor.

In certain embodiments, a fusion protein of the present disclosure (or an engineered host cell expressing the same) is used in combination with an inhibitor of CEACAM-1, CEACAM-3, CEACAM-5, or any combination thereof.

In certain embodiments, a fusion protein of the present disclosure (or an engineered host cell expressing the same) is used in combination with an agent that increases the activity (i.e., is an agonist) of a stimulatory immune checkpoint molecule. For example, a fusionprotein of the present disclosure (or an engineered host cell expressing the same) can be used in combination with a CD137 (4-1BB) agonist (such as, for example, urelumab), a CD134 (OX-40) agonist (such as, for example, MEDI6469, MEDI6383, or MEDI0562), lenalidomide, pomalidomide, a CD27 agonist (such as, for example, CDX-1127), a CD28 agonist (such as, for example, TGN1412, CD80, or CD86), a CD40 agonist (such as, for example, CP-870,893, rhuCD40L, or SGN-40), a CD122 agonist (such as, for example, IL-2) an agonist of GITR (such as, for example, humanized monoclonal antibodies described in PCT Patent Publication No. WO 2016/054638), an agonist of ICOS (CD278) (such as, for example, GSK3359609, mAb 88.2, JTX-2011, Icos 145-1, Icos 314-8, or any combination thereof). In any of the embodiments disclosed herein, a method may comprise administering a fusion protein of the present disclosure (or an engineered host cell expressing the same) with one or more agonist of a stimulatory immune checkpoint molecule, including any of the foregoing, singly or in any combination.

In certain embodiments, a combination therapy comprises a fusion protein of the present disclosure (or an engineered host cell expressing the same) and a secondary therapy comprising one or more of: an antibody or antigen binding-fragment thereof that is specific for a cancer antigen expressed by the non-inflamed solid tumor, a radiation treatment, a surgery, a chemotherapeutic agent, a cytokine, RNAi, or any combination thereof.

In certain embodiments, a combination therapy method comprises administering a fusion protein and further administering a radiation treatment or a surgery. Radiation therapy is well-known in the art and includes X-ray therapies, such as gamma-irradiation, and radiopharmaceutical therapies. Surgeries and surgical techniques appropriate to treating a given cancer or non-inflamed solid tumor in a subject are well-known to those of ordinary skill in the art.

In certain embodiments, a combination therapy method comprises administering a fusion protein of the present disclosure (or an engineered host cell expressing the same) and further administering a chemotherapeutic agent. A chemotherapeutic agent includes, but is not limited to, an inhibitor of chromatin function, a topoisomerase inhibitor, a microtubule inhibiting drug, a DNA damaging agent, an antimetabolite (such as folate antagonists, pyrimidine analogs, purine analogs, and sugar-modified analogs), a DNA synthesis inhibitor, a DNA interactive agent (such as an intercalating agent), and a DNA repair inhibitor. Illustrative chemotherapeutic agents include, without limitation, the following groups: anti-metabolites/anti-cancer agents, such as pyrimidine analogs (5-fluorouracil, floxuridine, capecitabine, gemcitabine and cytarabine) and purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristin, vinblastin, nocodazole, epothilones and navelbine, epidipodophyllotoxins (etoposide, teniposide), DNA damaging agents (actinomycin, amsacrine, anthracyclines, bleomycin, busulfan, camptothecin, carboplatin, chlorambucil, cisplatin, cyclophosphamide, Cytoxan, dactinomycin, daunorubicin, doxorubicin, epirubicin, hexamethylmelamineoxaliplatin, iphosphamide, melphalan, merchlorehtamine, mitomycin, mitoxantrone, nitrosourea, plicamycin, procarbazine, taxol, taxotere, temozolamide, teniposide, triethylenethiophosphoramide and etoposide (VP 16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates -busulfan, nitrosoureas (carmustine (BCNU) and analogs, streptozocin), trazenes—dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (TNP470, genistein) and growth factor inhibitors (vascular endothelial growth factor (VEGF) inhibitors, fibroblast growth factor (FGF) inhibitors); angiotensin receptor blocker; nitric oxide donors; anti-sense oligonucleotides; antibodies (trastuzumab, rituximab); chimeric antigen receptors; cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin, irinotecan (CPT-11) and mitoxantrone, topotecan, irinotecan), corticosteroids (cortisone, dexamethasone, hydrocortisone, methylpednisolone, prednisone, and prenisolone); growth factor signal transduction kinase inhibitors; mitochondrial dysfunction inducers, toxins such as Cholera toxin, ricin, Pseudomonas exotoxin, Bordetella pertussis adenylate cyclase toxin, or diphtheria toxin, and caspase activators; and chromatin disruptors.

Cytokines are increasingly used to manipulate host immune response towards anticancer activity. See, e.g., Floros & Tarhini, Semin. Oncol. 42(4):539-548, 2015. Cytokines useful for promoting immune anticancer or antitumor response include, for example, IFN-α, IL-2, IL-3, IL-4, IL-10, IL-12, IL-13, IL-15, IL-16, IL-17, IL-18, IL-21, IL-24, and GM-CSF, singly or in any combination with the binding proteins or cells expressing the same of this disclosure.

In still further aspects, methods are provided for activating or stimulating an immune cell (i.e., a host cell) modified to express on its cell surface a fusion protein of the present disclosure, wherein the methods comprise contacting the modified immune cell with a strep-tag peptide (which, in some embodiments, comprises or consists of the amino acid sequence shown in SEQ ID NO:19), under conditions and for a time sufficient for the modified immune cell to be activated. In certain embodiments, the strep-tag peptide is located on the surface of a cell. In certain embodiments, the strep-tag peptide is contained in a cell surface protein, such as a cell surface receptor or a marker. In further embodiments, the cell surface receptor comprises a CAR or a TCR. In particular embodiments, the cell surface protein comprises from one to about five strep-tag peptides (e.g., one, two, three, four, five, or six strep-tag peptides). In additional aspects, methods are provided for activating or stimulating a modified immune cell, wherein the methods comprise contacting the modified immune cell with a binding protein that specifically binds to a strep-tag peptide on the cell surface of the modified immune cell, thereby activating or stimulating the modified immune cell, wherein the modified immune cell comprises (a) a first polynucleotide encoding a cell surface receptor optionally encoding the cell surface receptor containing a strep-tag peptide, wherein the cell surface receptor specifically binds to a target antigen; and (b) a second polynucleotide encoding a cell surface marker optionally encoding the cell surface marker containing a tag peptide, wherein the encoded strep-tag peptide optionally comprises or consists of the amino acid sequence shown in SEQ ID NO: 19, and provided that at least one of the cell surface receptor and the cell surface marker contain the strep-tag peptide. By way of illustration, an example of activating or stimulating a modified immune cell comprises contacting (a) an anti-CD19 CART cell that expresses on its cell surface a strep-tag peptide of SEQ ID NO:19 (e.g., expressed as a fusion with the CAR or as a fusion with a transduction marker such as EGFRt) with (b) a binding protein (e.g., an antibody or antigen-binding fragment thereof, optionally comprised in a fusion protein) that specifically binds to the strep-tag peptide.

In certain embodiments, the cell surface receptor comprises the tag peptide. In further embodiments, the cell surface receptor comprises a CAR or a TCR. In other embodiments, the cell surface marker comprises the tag peptide. In certain embodiments, both the cell surface receptor and the cell surface marker comprise a strep-tag peptide.

In certain embodiments, the modified host cell to be activated or stimulated comprises an immune cell (e.g., a T cell, NK cell, or NK-T cell). In certain embodiments, the cell surface receptor is or comprises a CAR or a TCR. In further embodiments, the marker comprises EGFRt, CD19t, CD34t, or NGFRt. In particular embodiments, the modified immune cell is activated or stimulated multiple times, and may be activated or stimulated in vitro, ex vivo, or in vivo.

### EXAMPLES

### Example 1

### Design and Expression f ANTI-STII and STII-Tagged CARs

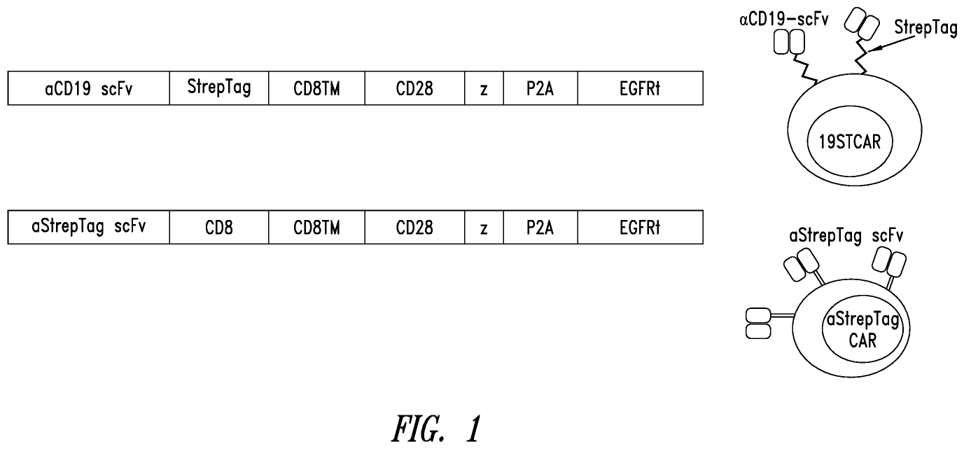

Tagged CARs for use in adoptive immunotherapy are described in PCT Publication WO 2015/095895. To investigate whether cells expressing such CARs could be selectively targeted for ablation, expression constructs encoding anti-CD19-STII (SEQ ID NO:19)(tagged CARs) and anti-STII CARs were generated. Exemplary constructs are shown in FIG. 1 (top left and bottom left), with schematic diagrams of cells expressing the encoded CARs shown at right.

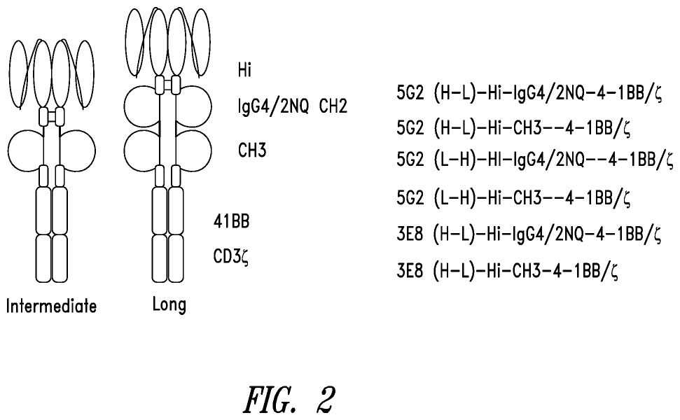

Additional anti-STII CARs were produced using scFvs (VH-VL and VL-VH orientations; SEQ ID NOs 5, 6, 11, and 12) derived from murine anti-STII monoclonal antibodies 5G2 and 3E8. The scFv sequences were subcloned into 4-1BB-CD3t CAR vectors having intermediate-length (IgG4 CH3 only) or long (IgG4CH2_{N297Q}CH3) immunoglobulin spacer domains, to produce the CAR designs shown in FIG. 2.

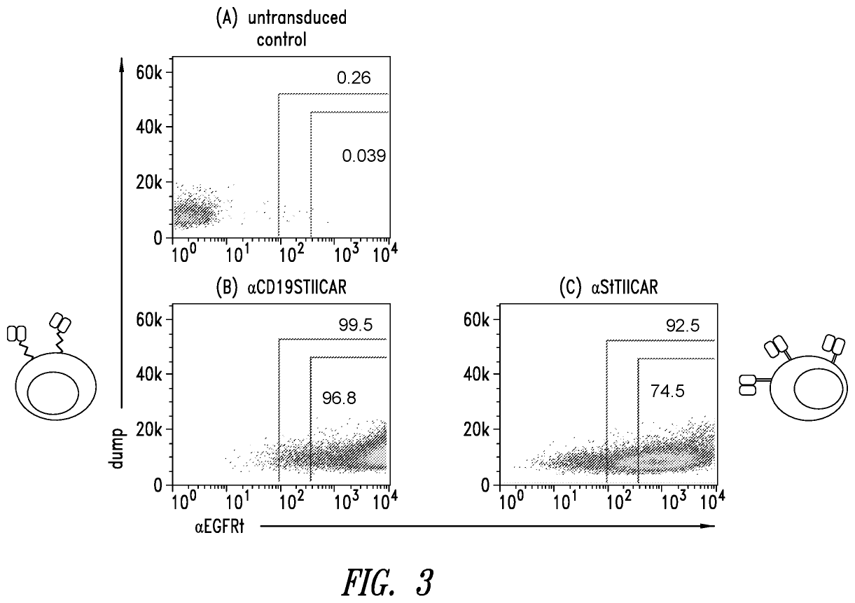

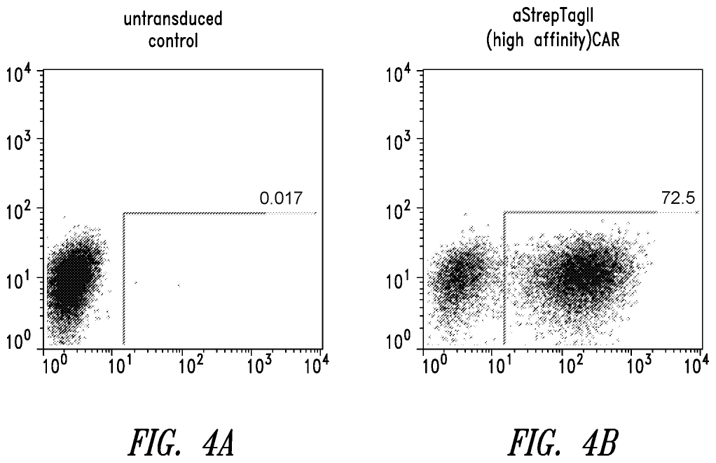

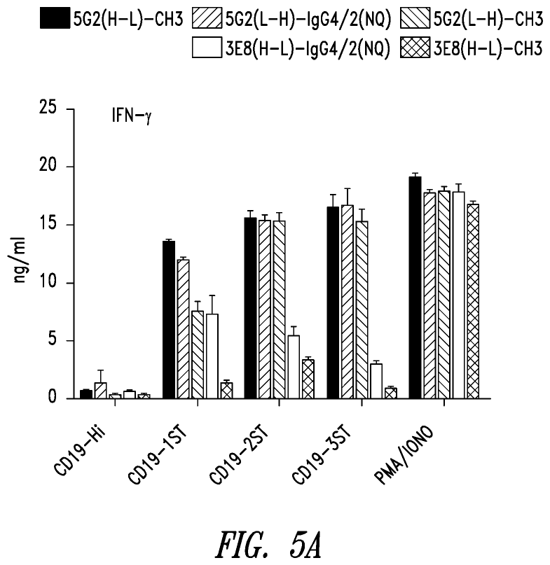

Next, primary PBMCs were transduced with the CAR constructs shown in FIG. 1 and expression assays were performed. Briefly, cells were analyzed by flow cytometry with detection of EGFRt transduction marker using a biotinylated anti-EGFR antibody and streptavidin PE). Both the anti-CD19-STII and anti-STII CARs showed robust expression (FIG. 3; “B” and “C”). An additional high-affinity anti-STII CAR was also expressed in primary PBMCs (FIG. 4B).

### Example 2

### Functional Characterization of ANTI-STII CAR T Cells

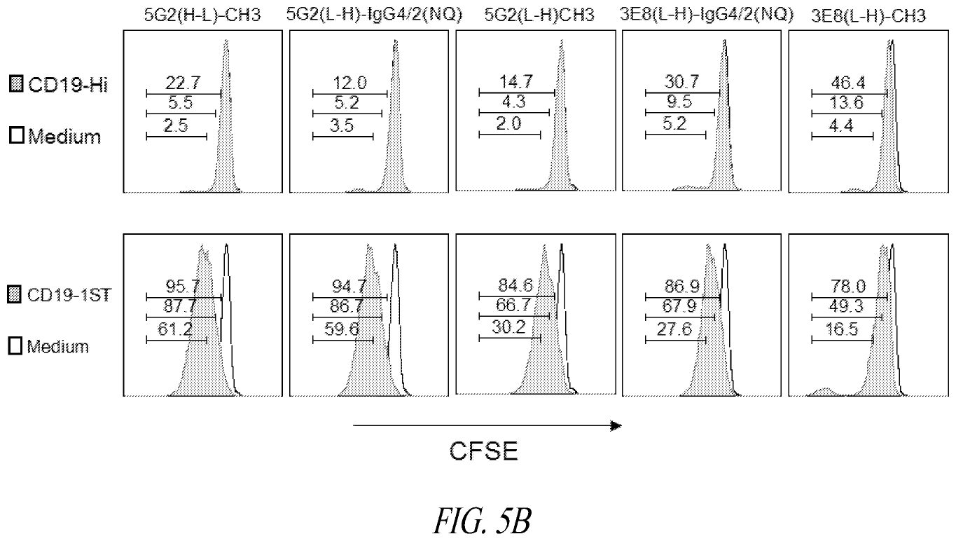

Next, the anti-STII CAR constructs shown in FIG. 2 were tested for recognition of STII-tagged CAR T cells. Briefly, human T cells were transduced with the anti-STII CAR constructs and incubated with anti-CD19-STII CAR T cells (one, two, or three STII peptide tags contained in the anti-CD19 CARs) or with control anti-CD19 CAR T cells (no STII). T cells stimulated with PMA/Ionomycin were used as a positive control. At 24 hours, culture supernatants were examined for IFN-γ by ELISA. As shown in FIG. 5A, all of the anti-STII CAR T cells produced cytokines in response to the target CAR T cells. Anti-STII CAR T cells with 5G2 scFv binding domains produced the greatest amounts of cytokines, while 3E8-based CAR T cells produced lower amounts. Reactivity appeared to increase with the number of STII peptides present in the target.

A proliferation assay was performed to investigate expansion of anti-STII CAR T cells in response to the tagged target cells. Anti-STII CAR T cells were labeled with carboxyfluorescein succinimidyl ester and stimulated with control anti-CD19 CAR T cells (no STII) or anti-CD19-STII CAR T cells. Cells were analyzed by flow cytometry 3 days after stimulation. Results are shown in FIG. 5B. All anti-STII CAR T cells tested proliferated in response to stimulation, with 5G2-based anti-STII CART cells expanding more than 3E8-based anti-STII CAR T cells.

### Example 3

### In Vitro Cytolytic Activity of ANTI-STII CAR T Cells

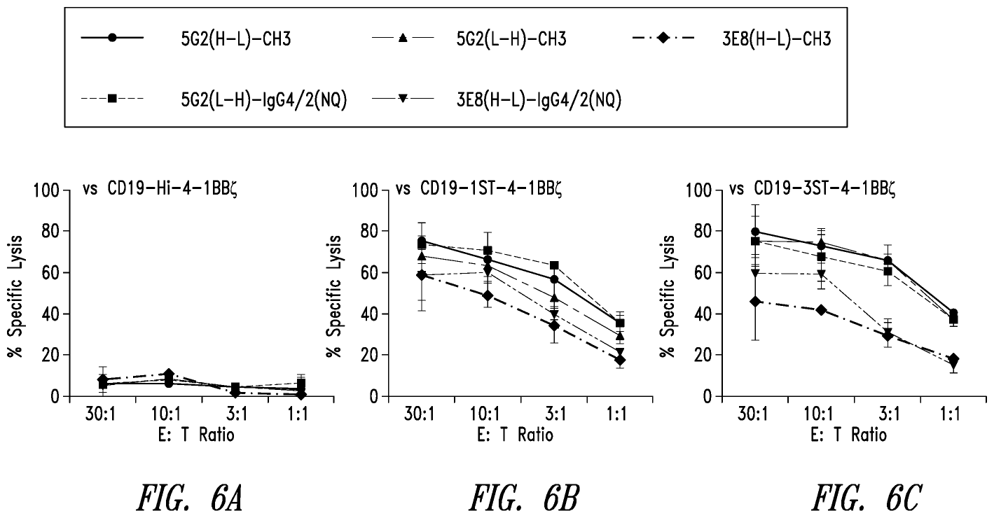

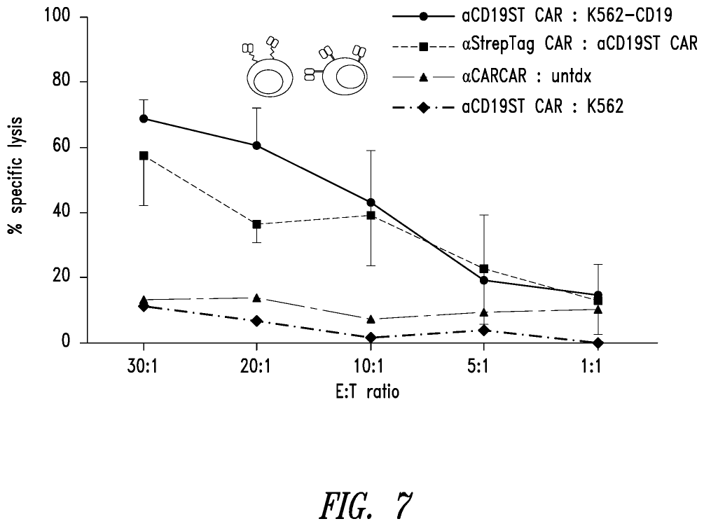

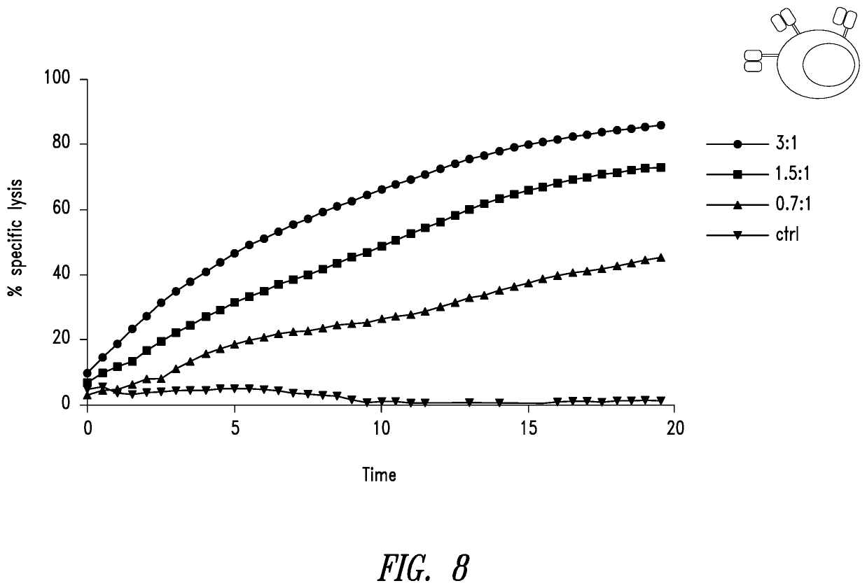

In vitro cytotoxicity assays were performed to measure specific killing activity of the anti-STII CAR T cells. In one experiment, Cr⁵¹-labeled target cells (control anti-CD19 CAR T; anti-CD19-1STII; anti-CD19-3STII) were co-cultured (4h) with anti-STII CART cells at various effector:target ratios (30:1, 10:1, 3:1, 1:1). Specific lysis was then determined by chromium release using a standard formula. Data are shown in FIGS. 6A-6C. All of the tested anti-STII CAR T cells had cytolytic activity against the target cells, with 5G2-based cells having the strongest activity. Notably, 5G2-based anti-STII CART cells lysed approximately 40% of anti-CD19-3STII cells at 1:1 E:T.

In another experiment, killing activity of anti-STII CAR T cells (against anti-CD19-STII CAR T cells) and anti-CD19-STII CAR T cells (against CD19⁺ K562 cells) were tested. Briefly, PBMCs were stimulated for 2 days with an anti-CD3/anti-CD28 stimulation reagent, followed by y-retroviral transduction with the CAR constructs. Specific lysis (Y-axis) was measured using the Europium TDA release assay (Perkin Elmer) via ELISA according to manufacturer's instructions. As shown in FIG. 7, both groups of CAR T cells exhibited killing activity against their respective targets in a dose-dependent manner. In a third experiment, killing activity was measured following longer co-incubation of anti-STII (effector) and anti-CD19-STII CAR—expressing cells (target). PBMCs were stimulated for 2 days with anti-CD3/anti-CD28, and primary cells were transduced with the anti-STII CAR constructs. HEK293 cells expressing an anti-CD19-STII CAR were used as the target. Cells were co-incubated for 20 h, and lysis of target cells was measured via impedance using the xCELLIGENCE RTCA assay (ACEA Biosciences, Inc., San Diego, Calif.). The killing capacity of anti-STII CAR T cells increased in a time-dependent and dose-dependent manner (FIG. 8).

### Example 4

### Construction and Testing of ANTI-STII CARS for In Vivo Animal Studies

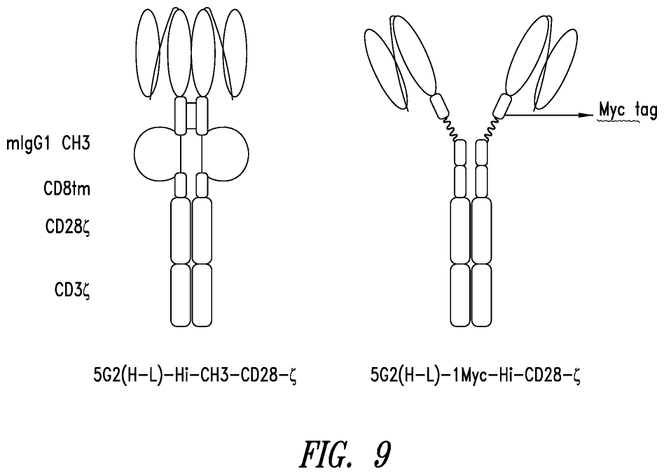

To determine whether the in vitro results described in Example 3 can inform therapies for human application, in vivo animal studies are needed. To this end, anti-STII CARs were generated using murine components to reduce the risk of immunogenicity when administered to a mouse model. Briefly, the 5G2 scFv (V_{H}-V_{L} configuration) was subcloned into CAR constructs with murine transmembrane and intracellular components and a spacer consisting of either: (1) a murine IgG1 CH3 domain (intermediate spacer); or (2) a single Myc tag+a G₄5 linker (short spacer). Exemplary CAR designs are shown in FIG. 9.

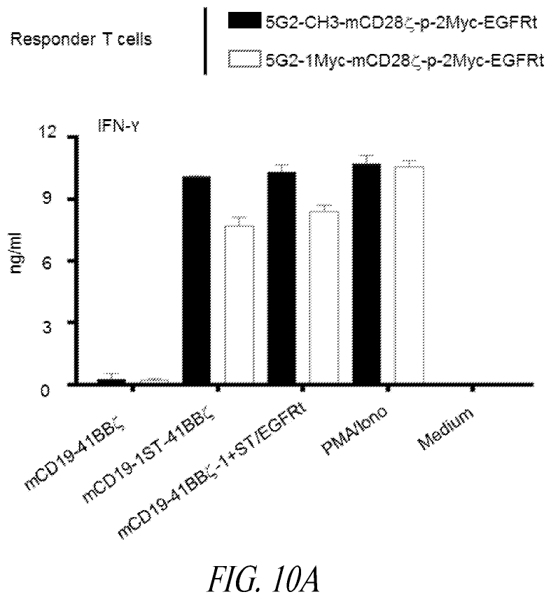

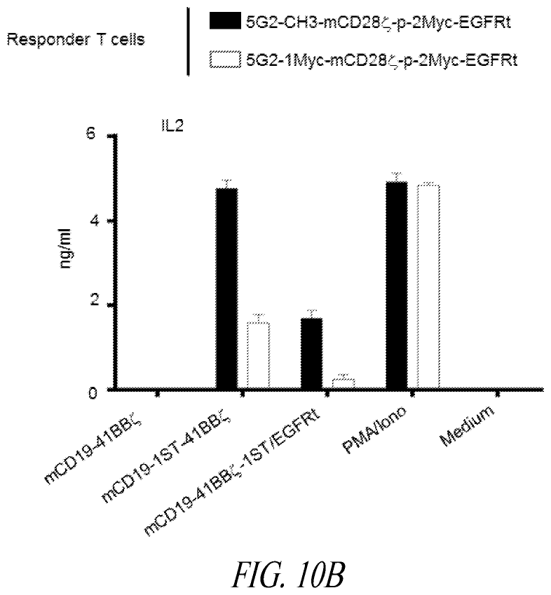

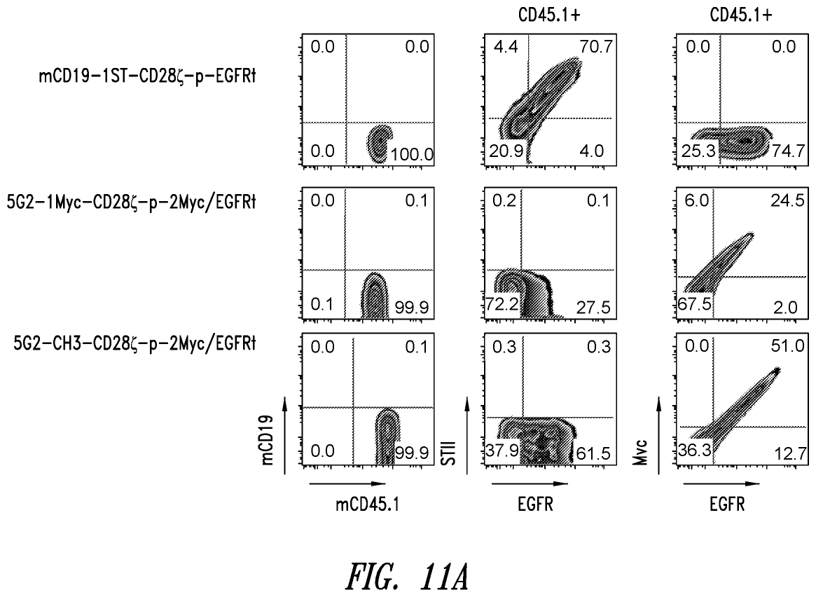

Next, mouse T cells were transduced to express the anti-STII CARs shown in FIG. 9. CAR expression was determined by staining for the 2Myc-EGFRt transduction marker, also encoded by the constructs. Anti-STII murine CAR T cells were incubated with mCD19-STII CAR T cells (having one copy of STII either contained in the CAR spacer region or fused to a co-expressed truncated EGFR) or with negative control cells (anti-CD19 CAR T, no STII). Non-treated and PMA/ionomycin-treated T cells were used as positive controls. Cytokine production (IFN-γ, FIG. 10A; IL-2, FIG. 10B) was measured at 24 hours. These data show that the anti-STII CARs redirected mouse T cell specificity to cells expressing cell surface STII (either as part of a CAR or in an EGFRt/STII fusion).

### Example 5

### Reduction of Tagged CAR T Side Effects Using ANTI-STII CAR T Cells

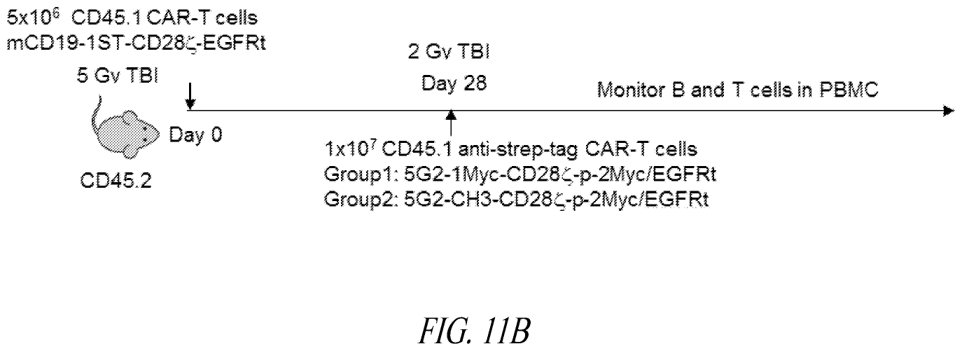

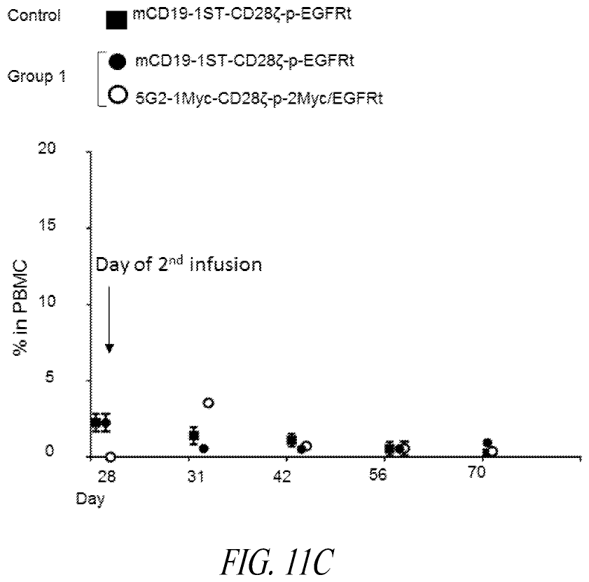

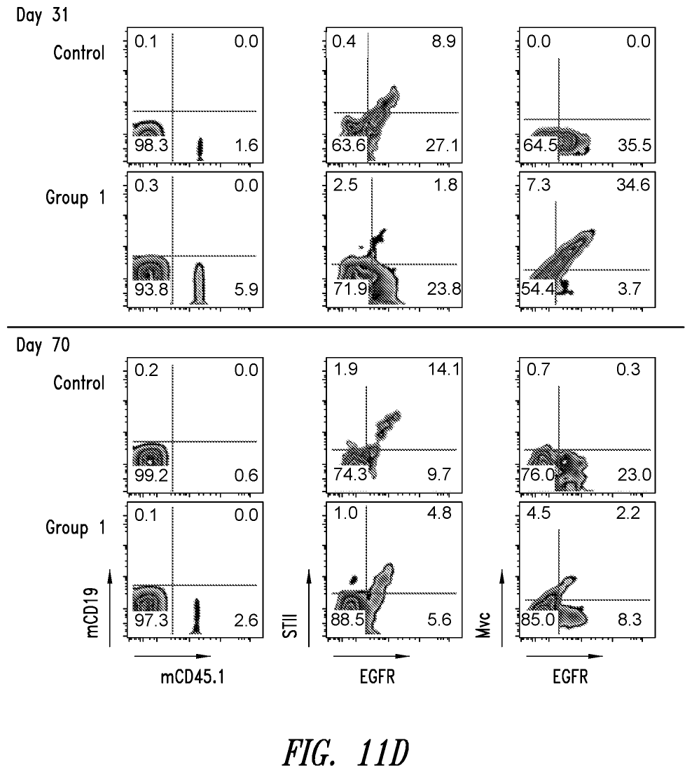

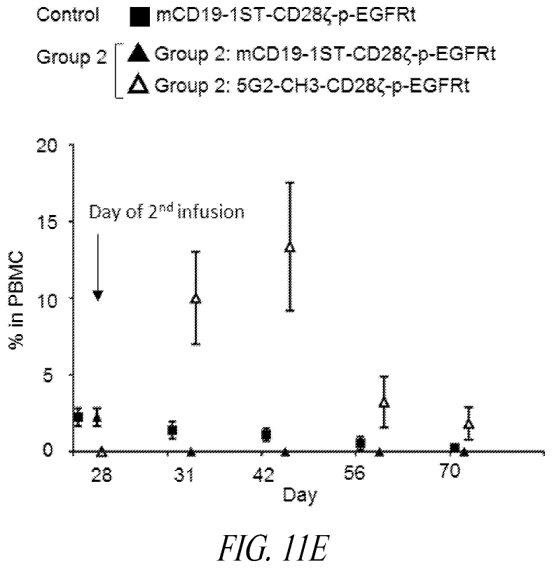

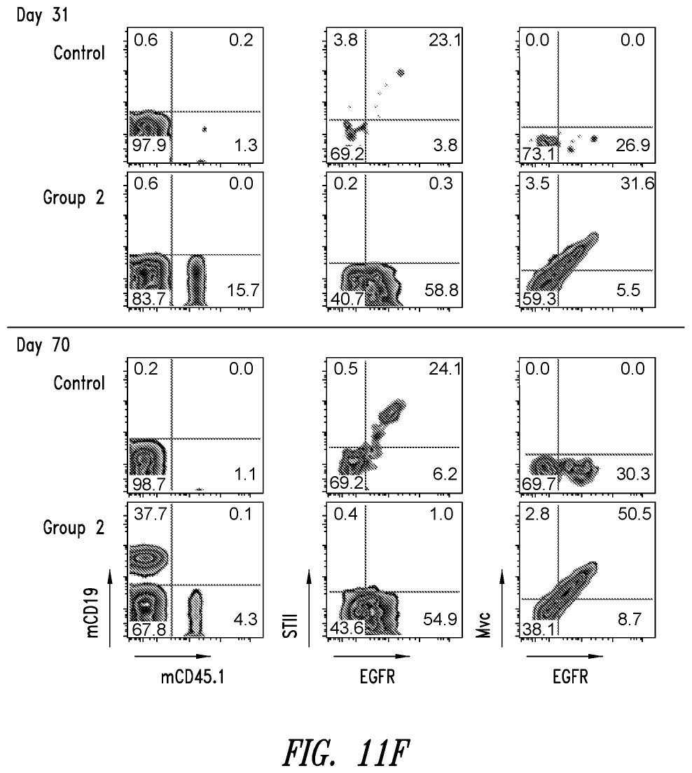

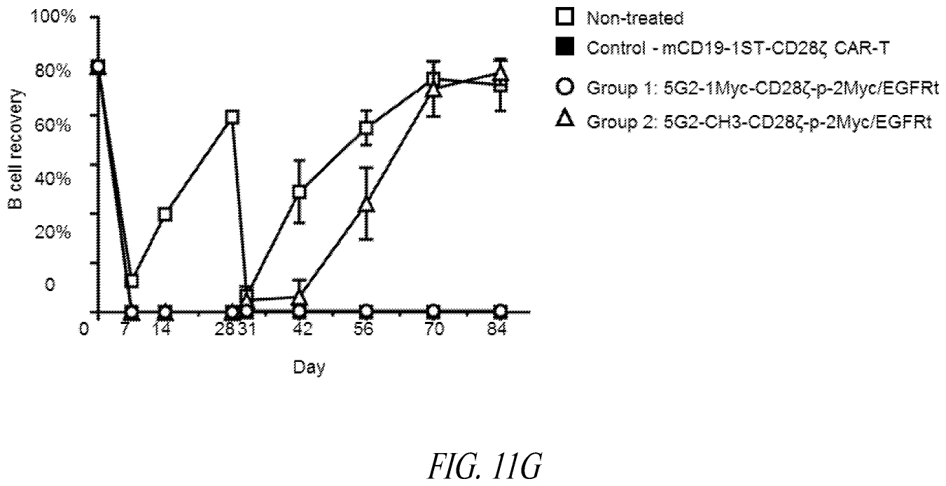

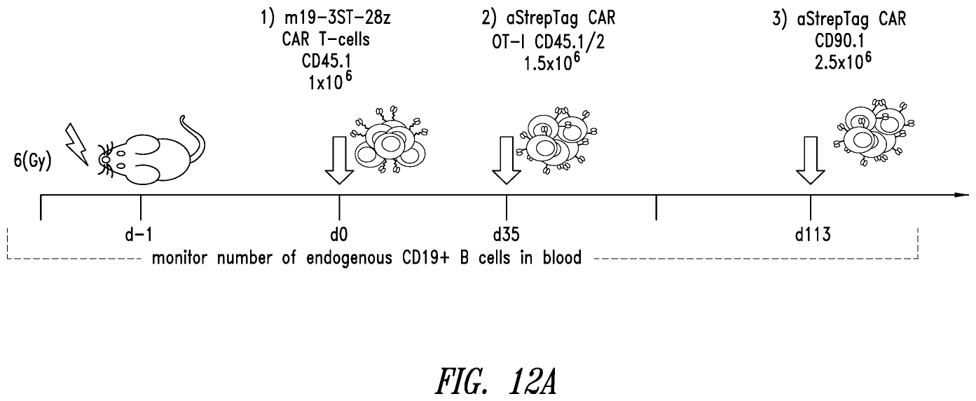

Next, the ability of anti-STII CART cells to eliminate STII-tagged CART cells in vivo and thereby reduce side effects from the tagged CAR T cells (in this case, to permit recovery from B cell aplasia following irradiation and treatment with tagged anti-CD19 CAR T cells) was investigated using a mouse model. First, cell surface expression of the CARs was examined. Briefly, CD45.1⁺ mouse T cells transduced with the CAR expression constructs were stained with mouse anti-CD19, anti-CD45.1, anti-EGFR, anti-STII, and anti-Myc monoclonal antibodies and analyzed by flow cytometry. Cell surface expression was confirmed for all CARs (FIG. 11A). Cells were then injected into CD45.2⁺ C57/B16 mice according to the treatment schedule shown in FIG. 11B. Briefly, all mice received 6Gy total body irradiation (TBI) at Day 0 and 2Gy (TBI) at day +27 to reduce B cell counts. Non-treated mice received radiation only and did not receive CART cells. Control mice received anti-CD19-1STII CART cells (Day +0), but did not receive anti-STII CAR T cells. Test mice were administered 5×10⁶CD45.1⁺ murine anti-CD19-STII-CD28ζ⁺ EGFRt⁺splenocytes at Day 0. At Day +28, test mice were transfused with 1×10⁷ CD45.1⁺ murine T cells expressing anti-STII CARs with a short (one Myc tag) spacer (treatment “Group 1”) or with an intermediate-length (CH3) spacer (treatment “Group 2”). T and B cell counts in PBMC were monitored by flow cytometry throughout the treatment schedule.

Data from the Group 1 mice is shown in FIGS. 11C and 11D. Briefly, the frequency of anti-CD19-1STII CART cells and of anti-STII CART cells was monitored at 28, 31, 42, 56, and 70 days after infusion with anti-STII CAR T cells. As shown in FIG. 11C, anti-STII cells with a Myc-tag spacer were effectively transferred in vivo and partially eliminated the anti-CD19-STII cells. B cell counts were also measured (at days +31 and +42 after infusion with anti-STII CAR T cells) by flow cytometry. FIG. 11D shows that treatment with T cells expressing anti-STII CAR with a short (Myc tag) spacer did not reverse B cell aplasia in the mice.

Data from the Group 2 mice is shown in FIGS. 11E and 11F. Frequencies of anti-CD19-1STII CART cells and anti-STII CART cells in PBMC were monitored at 28, 31, 42, 56, and 70 days after infusion with the anti-STII CAR T cells. As shown in FIG. 11E, anti-STII cells with a Myc-tag spacer were effectively transferred in vivo and eliminated the anti-CD19-STII cells. B cell counts were also measured (at days +31 and +42 after infusion with anti-STII CAR T cells) by flow cytometry. FIG. 11F shows that treatment with T cells expressing anti-STII CAR with an intermediate spacer effectively reversed B cell aplasia in the mice (lower left-hand panel). B cell recovery data from the experimental treatment schedule is summarized in FIG. 11G.

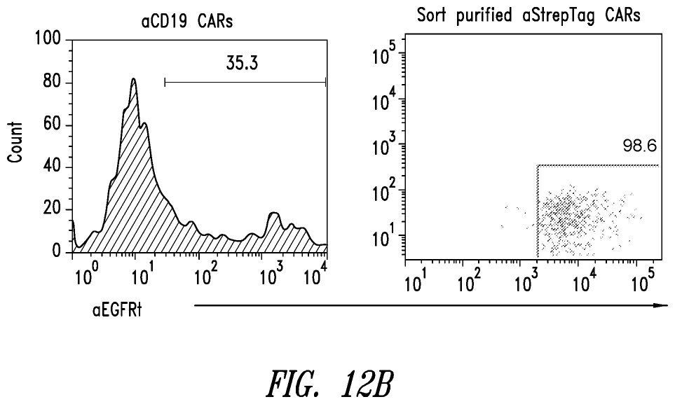

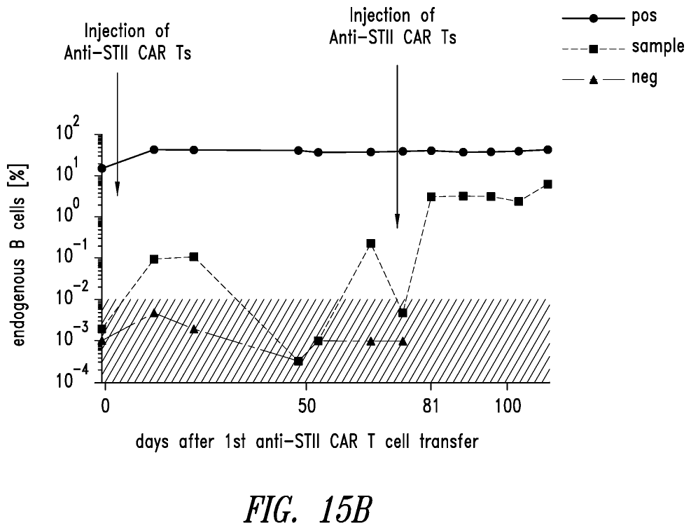

In a second experimental treatment shown in FIG. 12A, C57/B16 mice received sublethal radiation as described above, followed by transfusion with lx10⁶ murine CD45.1⁺ anti-CD19-3STII-28z CART cells at Day +0 to induce B cell aplasia. At Day 35, the mice received (1.5×10⁶ OT-1 CD45.1/2⁺ anti-STII CART cells, followed by 2.5×10⁶ OT-1 CD90.1⁺ anti-STII CART cells at Day 113. B cell counts were monitored by flow cytometry throughout. FIG. 12B shows expression of anti-CD19-3STII-28z CAR T cells (left) and sorting of purified anti-STII CAR T cells (right) prior to infusion. CAR T cell counts were monitored following the first anti-STII CAR T cell transfer, showing a sustained decrease in anti-CD19 CAR T cell counts (FIG. 15A). Endogenous B cell counts were also monitored and showed a marked recovery in treated (“sample”) versus untreated (“neg”) mice (FIG. 15B).

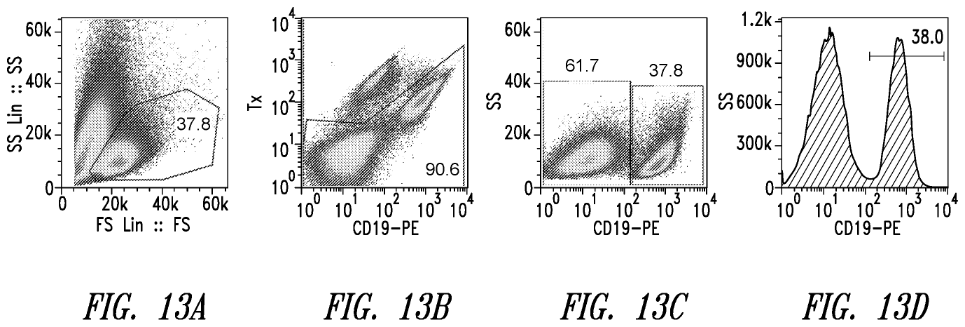

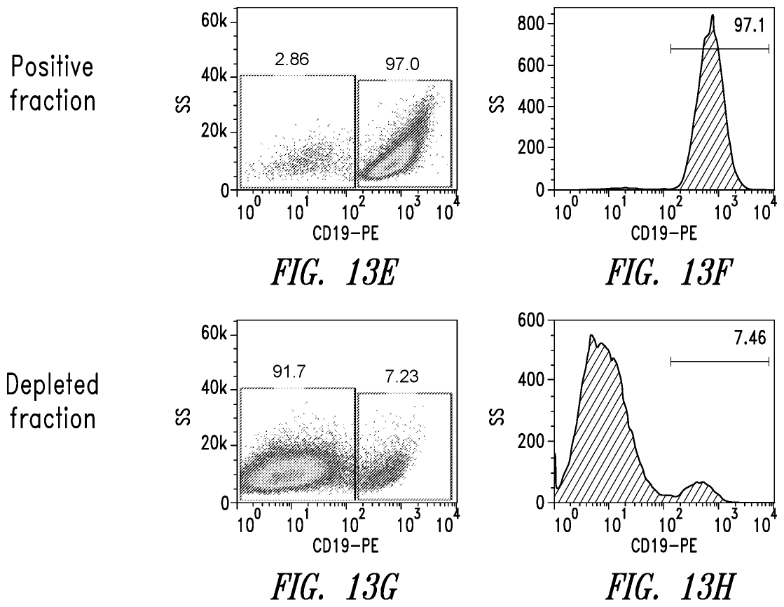

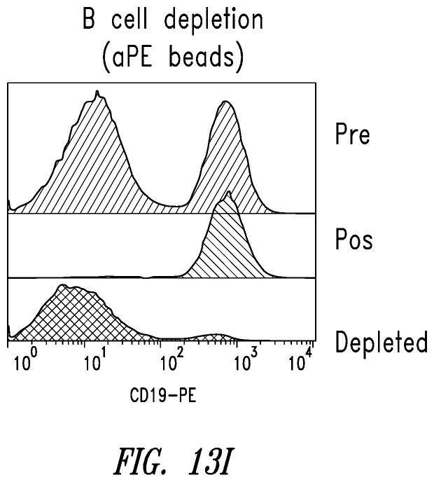

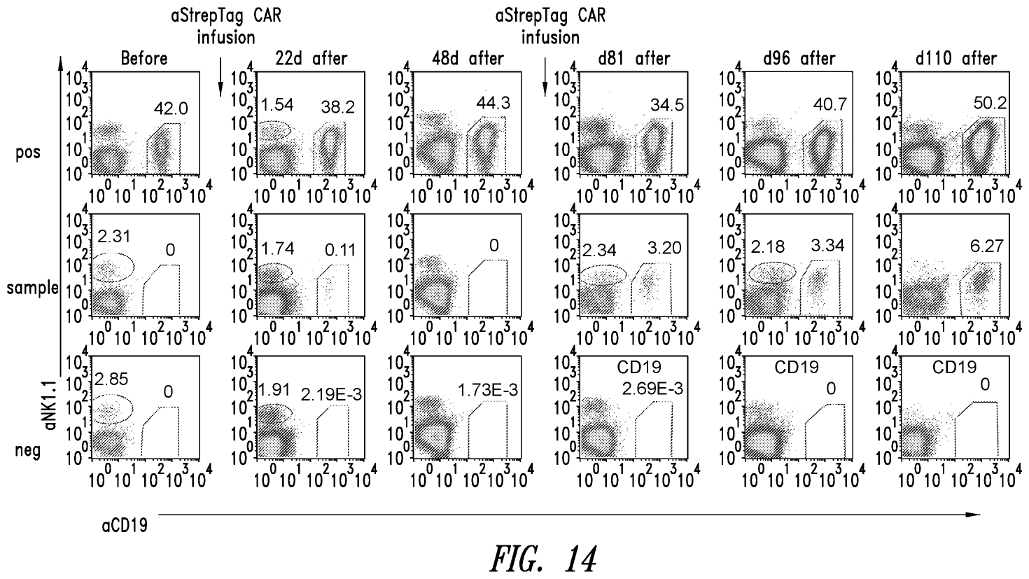

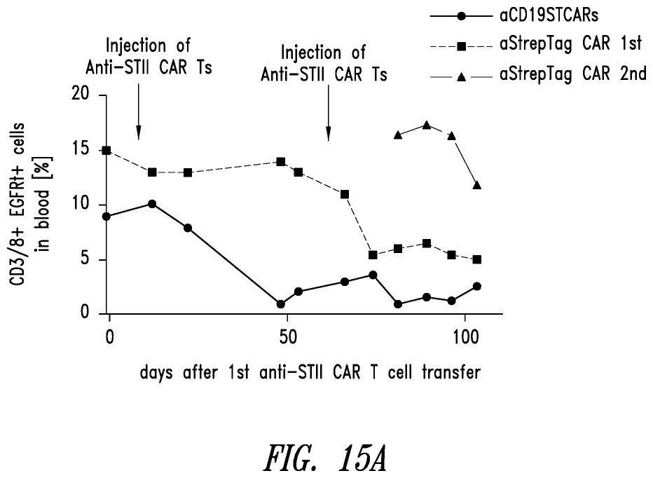

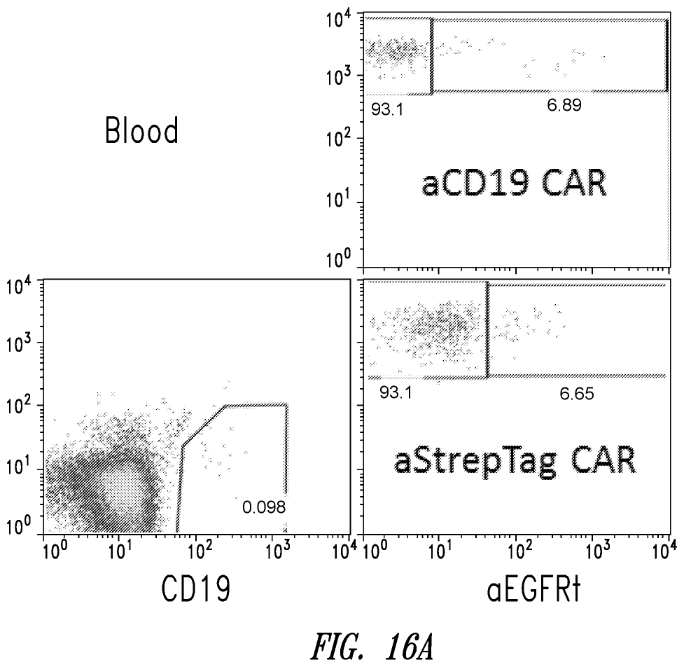

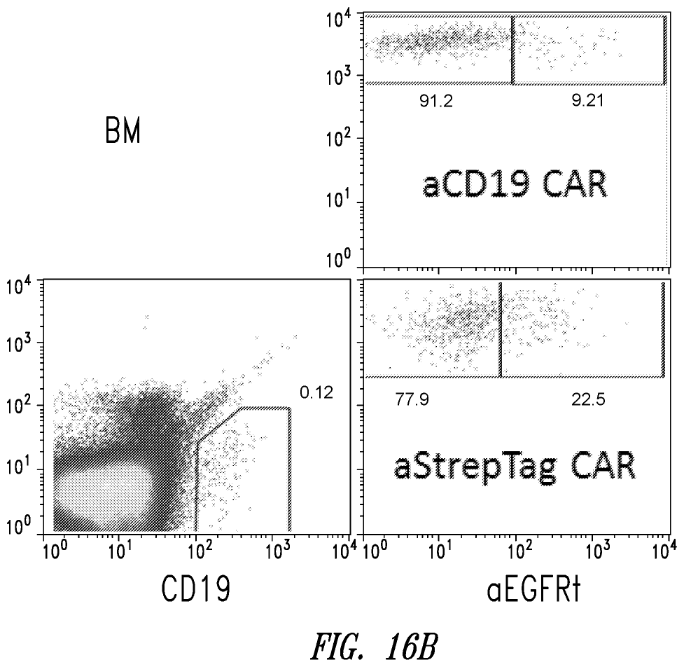

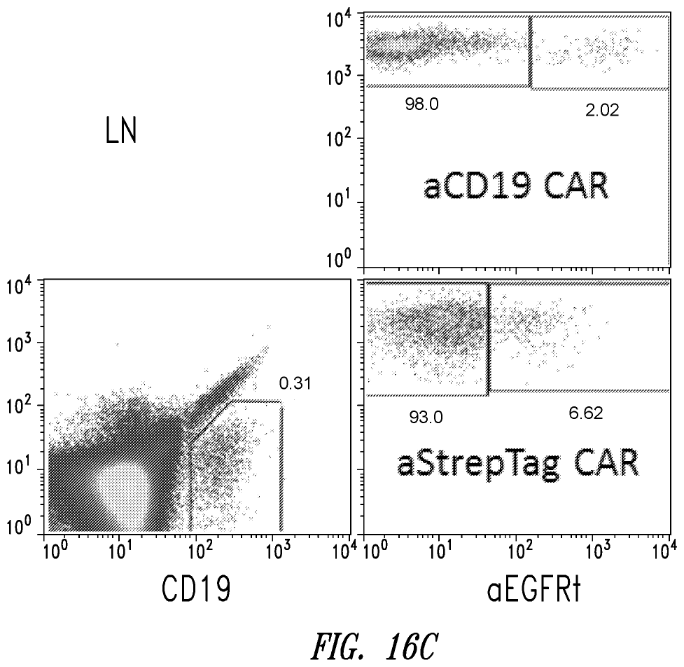

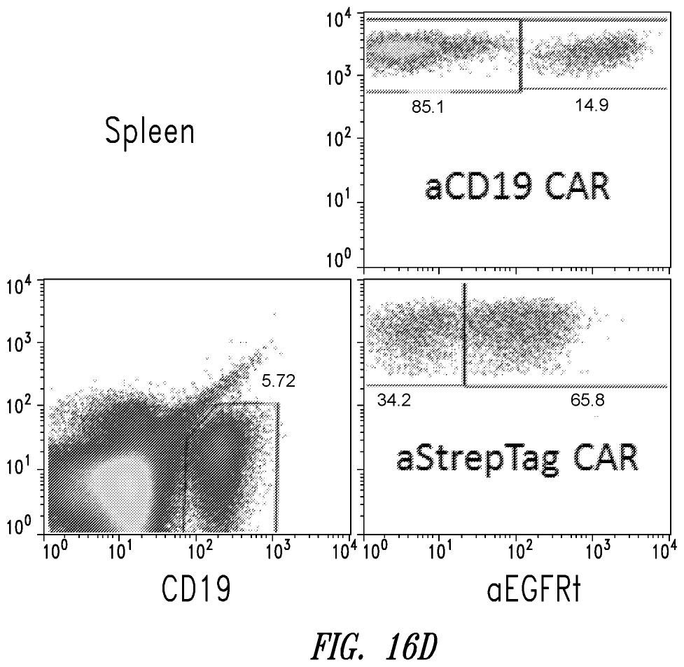

Depletion of endogenous B cells was confirmed following irradiation and anti-CD19-3STII CART cell infusion into the mice, but before transfer of anti-STII CAR T cells. Briefly, PBMCs were analyzed by flow cytometry with gating for live lymphocytes (FIG. 13A). Gated cells were then analyzed for CD19 expression using CD19PE by flow cytometry (13B-13H) or using anti-PE magnetic microbeads (Miltenyi Biotec) (FIG. 131). As shown in FIGS. 14-16D, infusion with anti-STII CAR T cells enabled recovery of the endogenous B cells (see “sample” data). Notably, endpoint analysis from primary tissues showed that CAR T cells and recovered B cells were largely present in the spleen and, to a lesser extent, lymph nodes (FIGS. 16A-16D).

### Example 6

### Uncoupling Expression of STII and CARS In Antigen-Specific T Cells

The tagged CAR T cells used in the preceding examples expressed STII as a part of the CAR molecule. However, the ability of tag peptides to be effectively displayed for recognition by anti-tag CAR T cells may be affected by the site of expression of the tag. To test this, an expression construct was designed to uncouple expression of the anti-CD19 CAR from that of the STII tag. Specifically, the STII-encoding sequence was fused to the 3′-end of the EGFRt-encoding sequence. The CAR and EGFRt:STII coding regions are separated by a self-cleaving peptide sequence so that the encoded CAR and EGFRt:STII proteins localize to the cell membrane as separate molecules. FIGS. 17B and 17E.

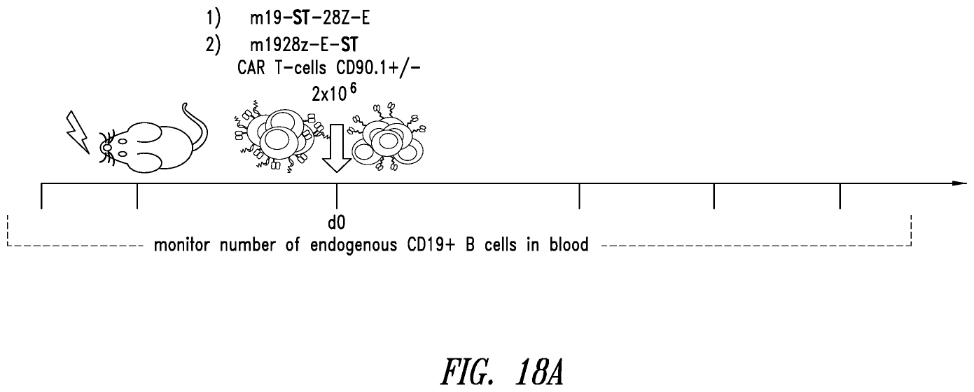

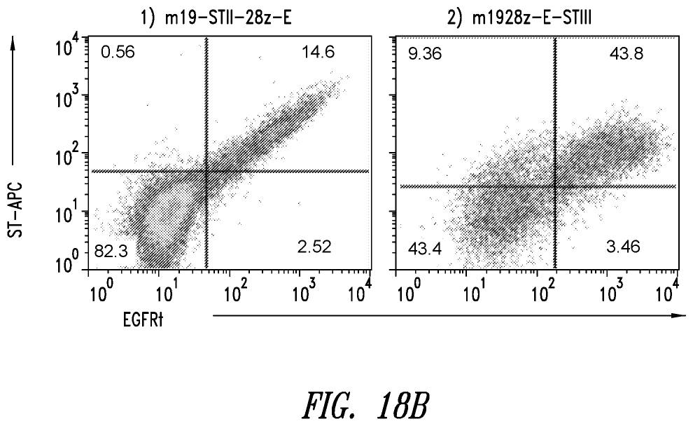

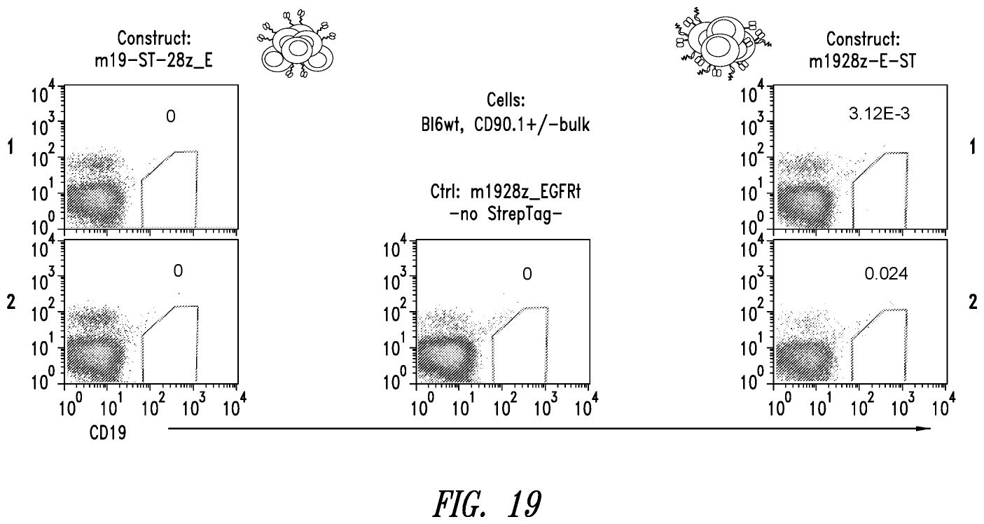

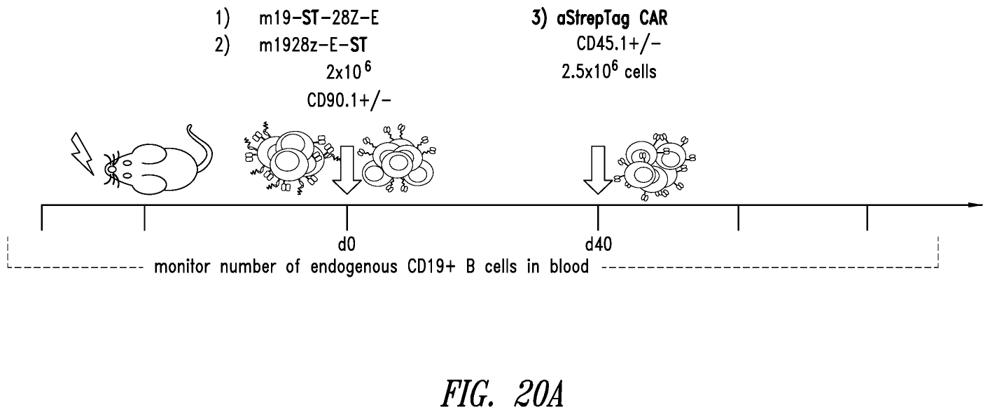

The two expression constructs (anti-CD19-3STII-28z_EGFRt and anti-CD19-28z_E-3STII) were tested for expression and activity in mice in an experiment illustrated in FIG. 18A. Briefly, mice received a sublethal dose of radiation and subsequently received 2×10⁶ C57/B16 CD90.1^{+/-} T cells transduced with either construct. Flow cytometry analysis showed that the cells with uncoupled CAR and STII expression (anti-CD19-28z E-3STII) expanded more efficiently than those expressing STII as part of the CAR. FIG. 18B. Treatment with CAR T cells expressing either construct reduced endogenous B cell counts. FIG. 19.

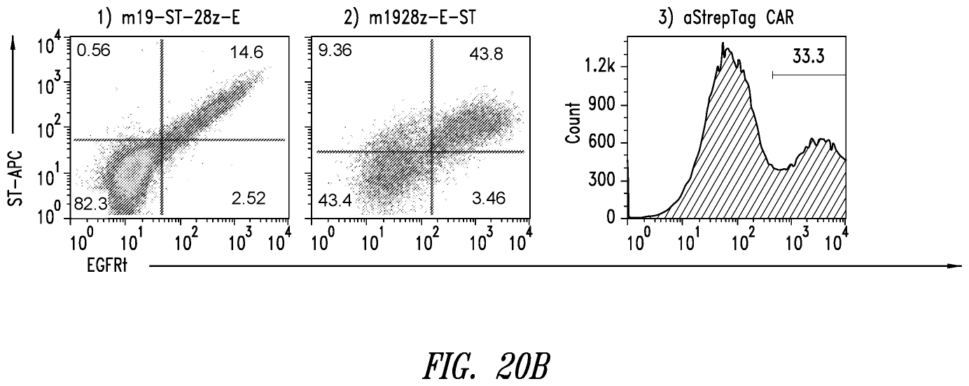

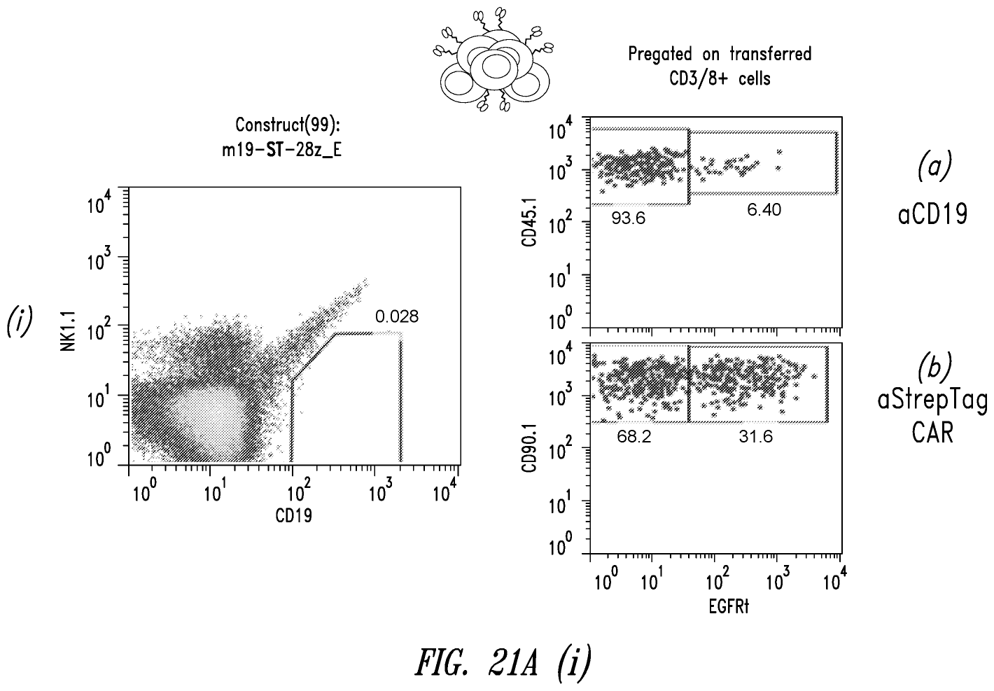

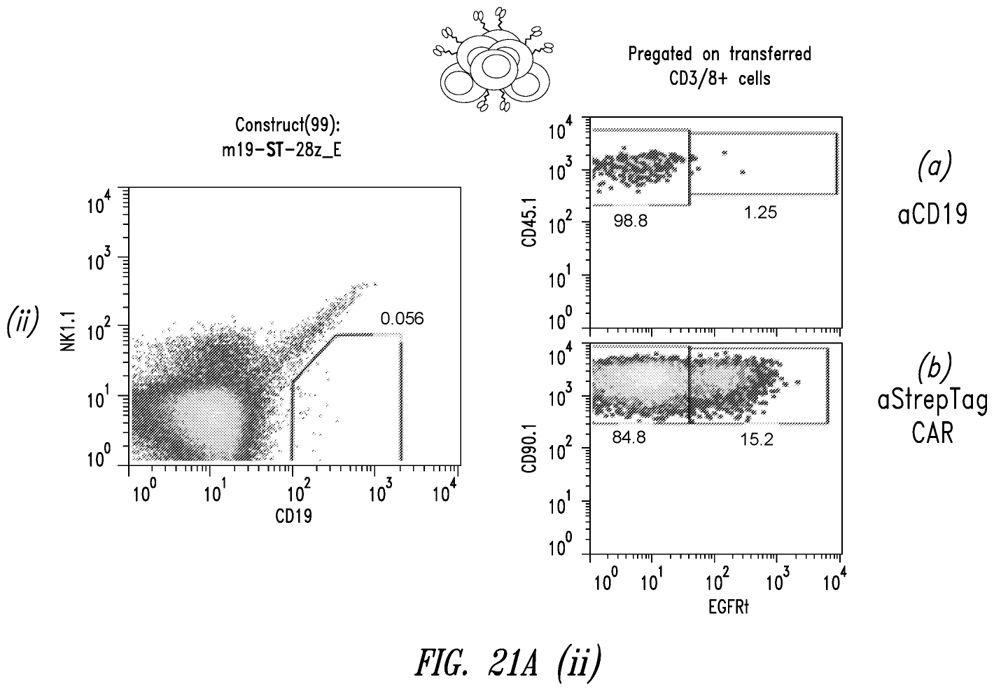

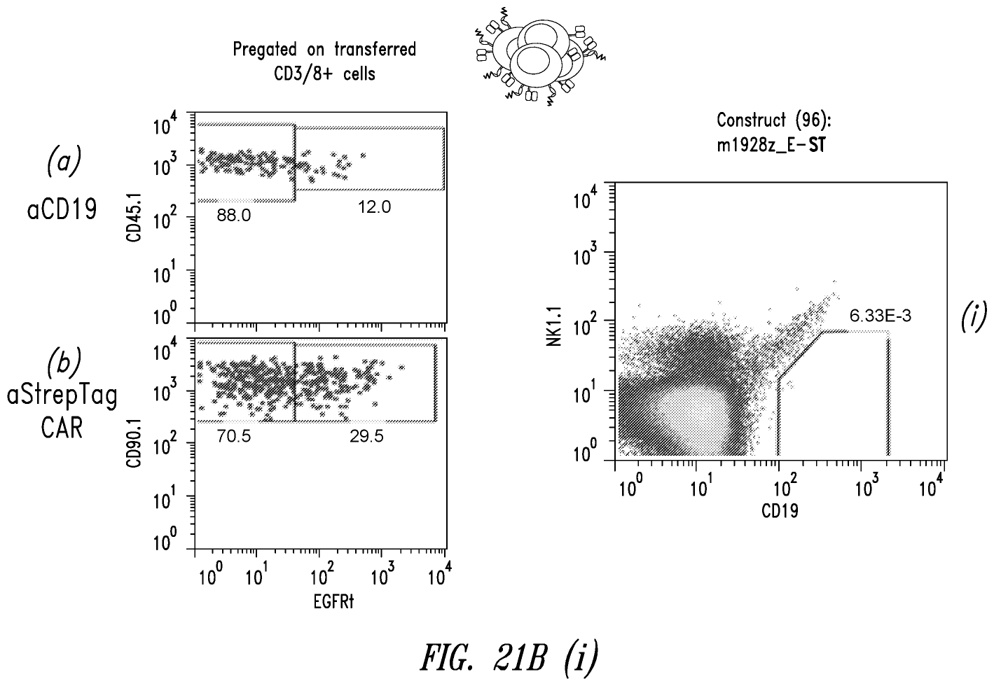

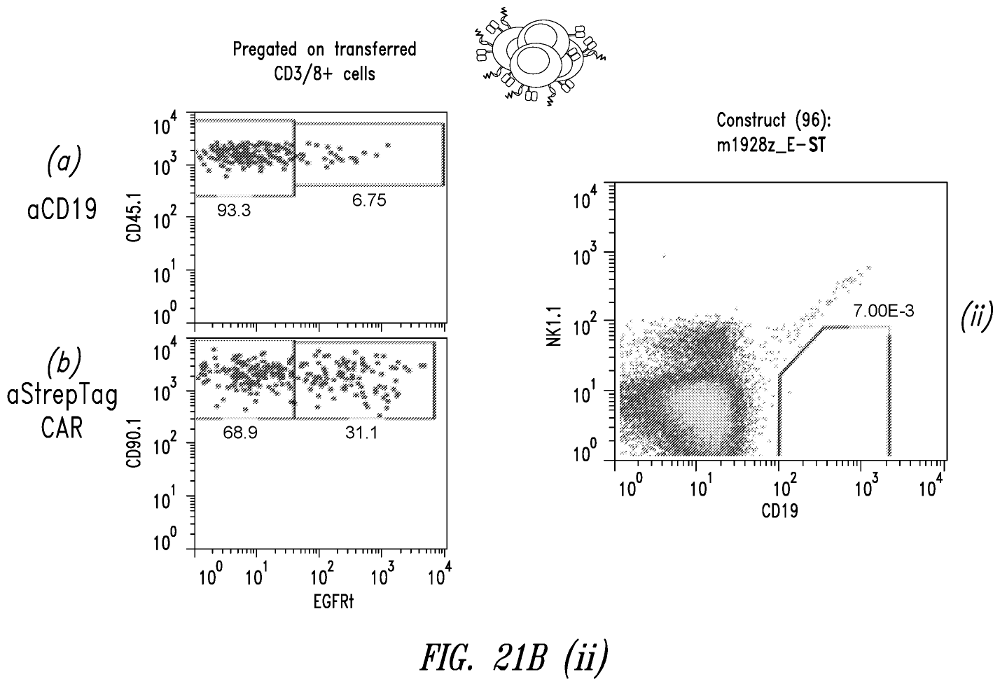

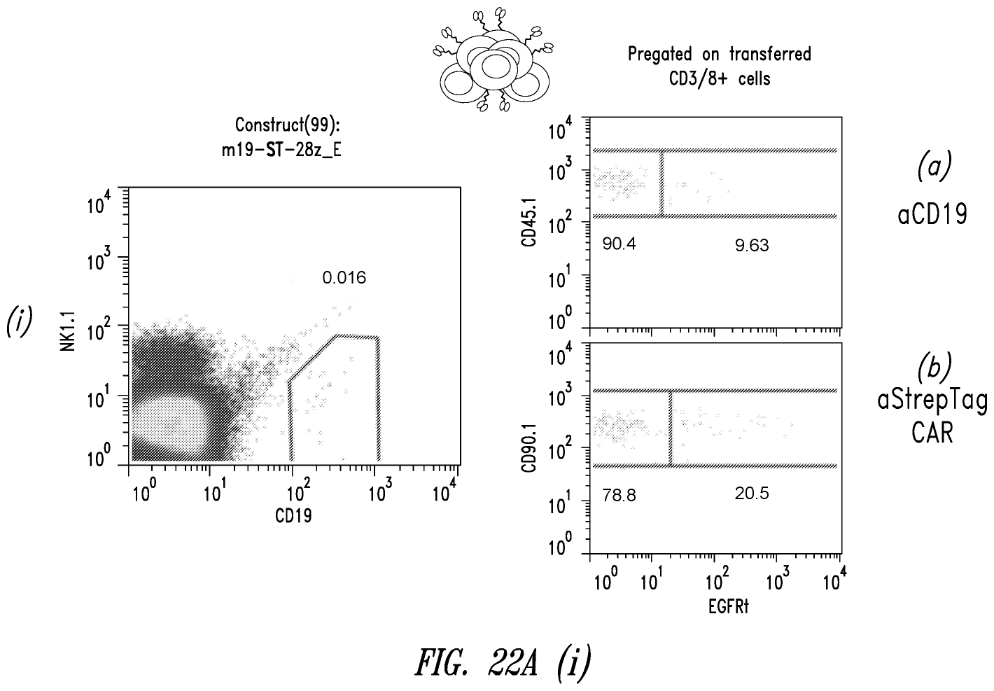

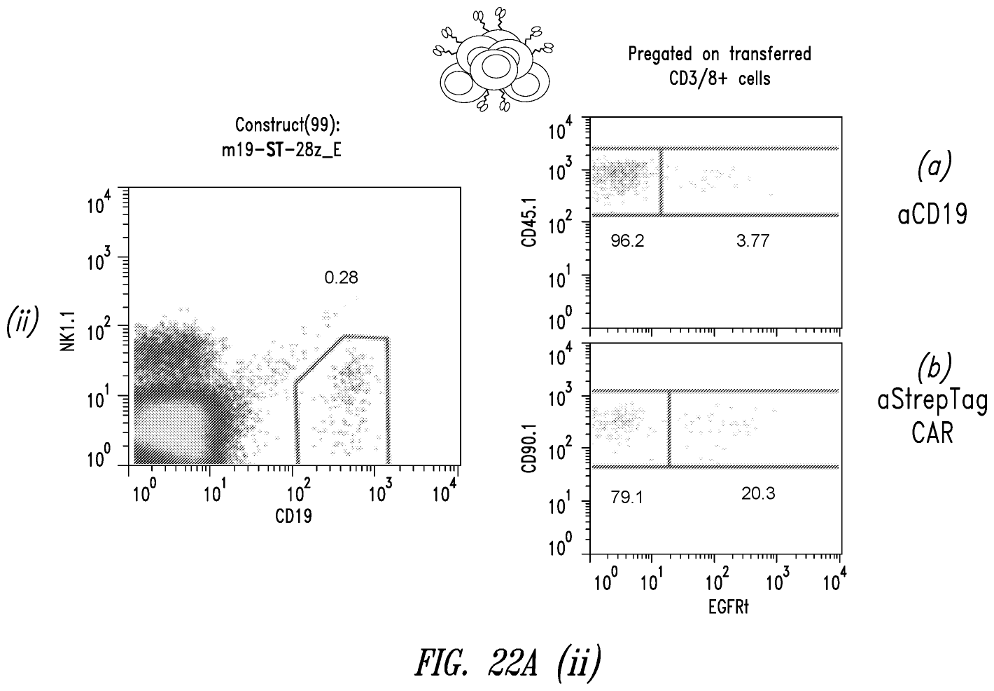

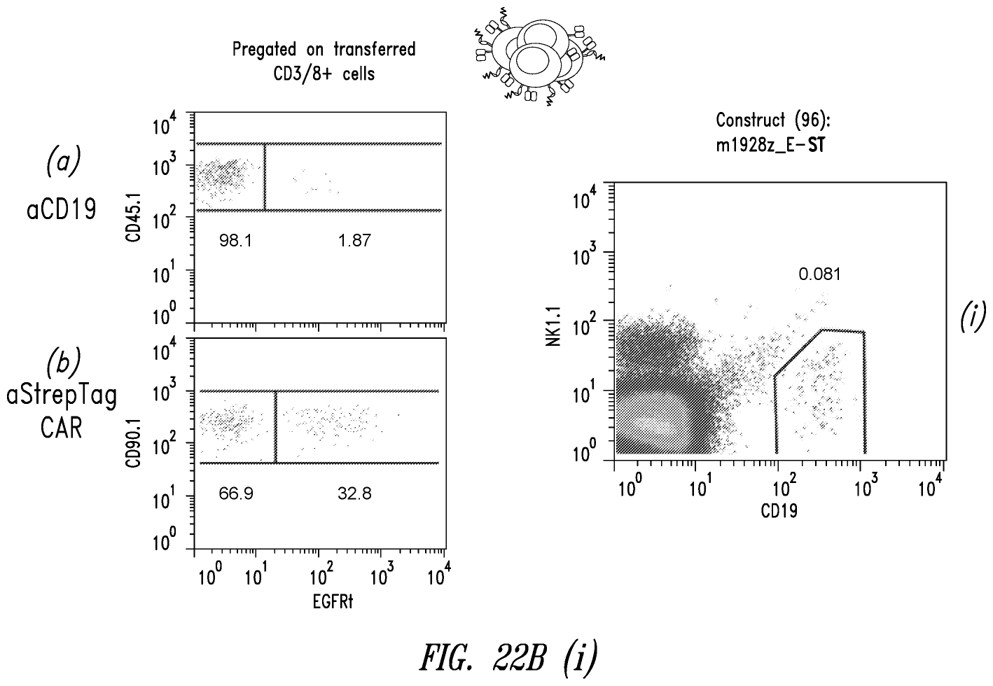

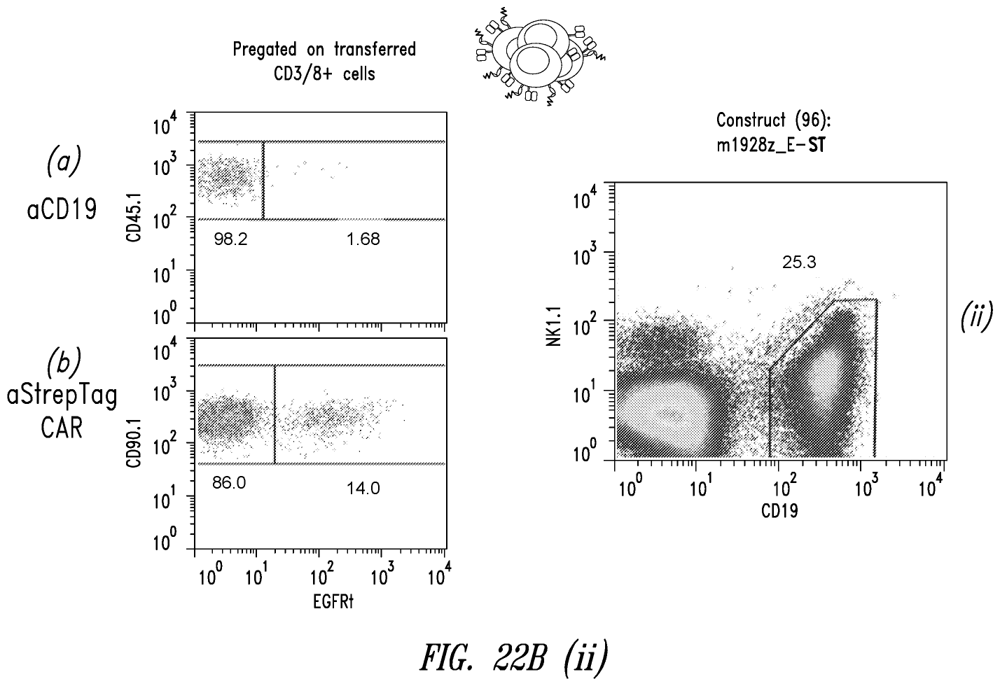

The effect of uncoupling STII and CAR expression on recognition by anti-STII CAR T cells was examined. Mice received a sublethal dose of radiation followed by injection of 2×10⁶ C57/B16 CD90.1^{+/-} T cells transduced with either CAR-STII construct at Day 0. At Day +40, the mice were infused with 2.5×10⁶ CD45.1^{+/-} anti-STII cells. FIG. 20A. Expression of the three CAR T cell types was confirmed (FIG. 20B). B cell counts were analyzed by flow cytometry at days +6 and +35 following injection of anti-STII CART cells (FIGS. 21A-21B and 22A-22B, respectively). Surprisingly, recovery of endogenous B cells was higher in mice that received the anti-CD19-28z_E-3STII CAR T cells, suggesting that uncoupling STII from the anti-CD19 CAR improved recognition and killing by anti-STII CAR T cells.

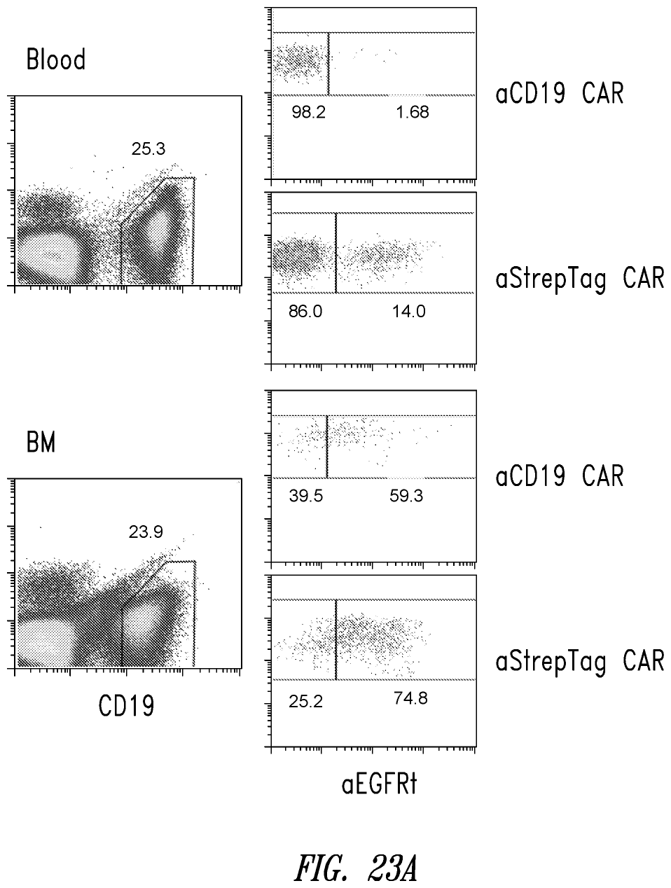

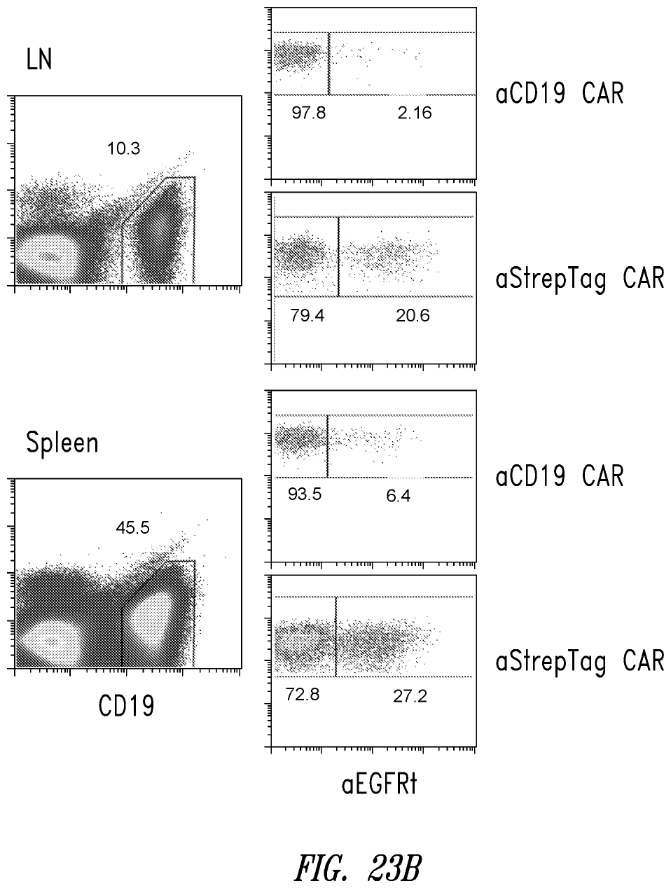

Endpoint analysis showed that recovered B cells were present in all primary tissues analyzed, and also that anti-STII CAR T cell counts were higher than those the tagged anti-CD19 CAR T cells. FIGS. 23A and 23B. Without wishing to be bound by theory, these data suggest that tagged antigen-specific T cells may be more effectively targeted by anti-tag CAR T cells when the tag and the antigen-specific receptor are expressed as separate molecules on the cell surface.

The various embodiments described above can be combined to provide further embodiments. All of the U.S. patents, U.S. patent application publications, U.S. patent applications, foreign patents, foreign patent applications and non-patent publications referred to in this specification and/or listed in US Provisional Patent Application No. 62/555,012 and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A fusion protein, comprising:
(a) an extracellular component comprising a binding domain that specifically binds to a strep-tag peptide;
(b) an intracellular component comprising an effector domain or a functional portion thereof; and
(c) a transmembrane domain connecting the extracellular and intracellular components.

2. The fusion protein of claim 1, wherein the binding domain is a scFv, scTCR, or ligand.

3. (canceled)

4. The fusion protein of claim 1, wherein the strep-tag peptide comprises or consists of the amino acid sequence shown in SEQ ID NO:19.

5. The fusion protein of claim 1, wherein the binding domain is a scFv comprising CDRs from 5G2 antibody, 3E8 antibody, or 4E2 antibody.

6. (canceled)

7. The fusion protein of claim 5, wherein the scFv comprises a light chain variable region (VL) that is at least 90% identical to the amino acid sequence shown in SEQ ID NO:10, 3, or 16; and a heavy chain variable region (V_{H}) that is at least 90% identical to the amino acid sequence shown in SEQ ID NO: 8, 2, or 14.

8. (canceled)

9. The fusion protein of claim 7, wherein the scFv comprises:
(a) a V_{L} of SEQ ID NO:10 and a V_{H} of SEQ ID NO:8;
(b) a V_{L} of SEQ ID NO:3 and a V_{H} of SEQ ID NO:2; or
(c) a V_{L} of SEQ ID NO:16 and a V_{H} of SEQ ID NO:14 .

10. The fusion protein of claim 9, wherein the scFv comprises or consists of:
(i) the amino acid sequence shown in SEQ ID NO:11 or 12;
(ii) the amino acid sequence shown in SEQ ID NO:5 or 6; or
(iii) the amino acid sequence shown in SEQ ID NO:17 or 18.

11.-12. (canceled)

13. The fusion protein of claim 1, wherein the intracellular component or the functional portion thereof comprises an Intracellular Tyrosine-based Activation Motif (ITAM).

14.-32. (canceled)

33. An isolated polynucleotide encoding a fusion protein of claim 1.

34.-39. (canceled)

40. A chimeric polynucleotide, comprising a first polynucleotide encoding a cell surface receptor, a second polynucleotide encoding a tagged marker, and a third polynucleotide encoding a self-cleaving polypeptide disposed between the first polynucleotide encoding the cell surface receptor and the second polynucleotide encoding the tagged marker, wherein:
(1) the first polynucleotide encoding the cell surface receptor comprises:
(a) an extracellular component comprising a binding domain that specifically binds a target antigen,
(b) an intracellular component comprising an effector domain or a functional portion thereof, and
(c) a transmembrane component connecting the extracellular component and the intracellular component; and
(2) the second polynucleotide encoding the tagged marker comprises a polynucleotide encoding the marker containing a tag peptide, wherein the encoded tag peptide comprises a strep-tag peptide.

41.-48. (canceled)

49. An expression construct, comprising the fusion protein-encoding polynucleotide of claim 33 operably linked to an expression control sequence.

50.-52. (canceled)

53. A host cell, comprising
the fusion protein-encoding polynucleotide of claim 33, wherein the host cell expresses the encoded fusion protein.

54.-60. (canceled)

61. A method for activating or stimulating an immune cell modified to express on its surface the fusion protein of claim 1, the method comprising contacting the modified immune cell with a strep-tag peptide, under conditions and for a time sufficient for the modified immune cell to be activated.

62.-66. (canceled)

67. A method for activating or stimulating a modified immune cell, the method comprising contacting the modified immune cell with a binding protein that specifically binds to a strep-tag peptide on the cell surface of the modified immune cell, thereby activating or stimulating the modified immune cell; wherein the modified immune cell comprises:
(a) a first polynucleotide encoding a cell surface receptor optionally encoding the cell surface receptor containing the strep-tag peptide, wherein the cell surface receptor specifically binds to a target antigen; and
(b) a second polynucleotide encoding a cell surface marker optionally encoding the cell surface marker containing the strep-tag peptide,
provided that at least one of the cell surface receptor and the cell surface marker contain the tag peptide.

68.-75. (canceled)

76. A method for targeted ablation of tagged cells, comprising administering to a subject an immune cell modified to express on its cell surface the fusion protein of claim 1, wherein the subject had been previously administered a tagged cell expressing a cell surface protein comprising a strep-tag peptide, thereby inducing a targeted immune response that ablates the tagged cells.

77.-88. (canceled)

89. A kit, comprising:
(a) an expression construct of claim 49; and
(b) reagents for transducing the expression construct of (a) into a host cell.

90. (canceled)

91. The fusion protein of claim 1, wherein the binding domain comprises:
(a) the heavy chain CDR 1 amino acid sequence shown in any one of SEQ ID NOs: 22, 28, or 34, or a variant of SEQ ID NO: 22, 28, or 34 having 1 to 3 amino acid substitutions and/or deletions;
(b) the heavy chain CDR 2 amino acid sequence shown in any one of SEQ ID NOs: 23, 29, or 35, or a variant of SEQ ID NO: 23, 29, or 35 having 1 to 3 amino acid substitutions and/or deletions; and
(c) the heavy chain CDR 3 amino acid sequence shown in any one of SEQ ID NOs: 24, 30, or 36, or a variant of SEQ ID NO: 24, 30, or 36 having 1 to 3 amino acid substitutions and/or deletions.

92. The fusion protein of claim 1, wherein the binding domain comprises:
(a) the light chain CDR 1 amino acid sequence shown in any one of SEQ ID NOs: 25, 31, or 37, or a variant of SEQ ID NO: 25, 31, or 37 having 1 to 3 amino acid substitutions and/or deletions;
(b) the light chain CDR 2 amino acid sequence shown in any one of SEQ ID NOs: 26, 32, or 38, or a variant of SEQ ID NO: 26, 32, or 38 having 1 or 2 amino acid substitutions and/or deletions; and
(c) the light chain CDR 3 amino acid sequence shown in any one of SEQ ID NOs: 27, 33, or 39, or a variant of SEQ ID NO: 27, 33, or 39 having 1 to 3 amino acid substitutions. and/or deletions.

93. An expression construct, comprising the chimeric polynucleotide of claim 40 operably linked to an expression control sequence.

94. A host cell, comprising the chimeric polynucleotide of claim 40, wherein the host cell expresses the encoded cell surface receptor and the encoded tagged marker.
